(19) United States
(12) Patent Application Publication
(10) Pub. No.: US 2011/0189155 A1
(43) Pub. Date: Aug. 4, 2011

(54) Large Form Of Human 2',5'-Oligoadenylate Synthetase OAS3 for Preventing or Treating Infection With Positive-Sense Single-Stranded RNA Viruses

(76) Inventors: Anne-Claire Brehin, Paris (FR); Anavaj Sakuntabhai, Paris (FR); Philippe Despres, Colombes (FR); Isabelle Casademont, Plaisir (FR); Cécile Julier, Paris (FR); Ampaiwan Chuansumrit, Bangkok (TH); Prida Malasit, Bangkok (TH); Sylvie Paulous, Sarcelles (FR)

(21) Appl. No.: 12/993,422

(22) PCT Filed: May 20, 2009
(86) PCT No.: PCT/IB2009/005956

§ 371 Date: Feb. 11, 2011

(30) Foreign Application Priority Data
May 20, 2008 (EP) 08290470.7

(51) Int. Cl. A61K 38/45; C12N 15/63; C12Q 1/68; C12N 5/10; C07H 21/00; A61K 31/7088; C12N 9/12; A61P 31/14

(52) U.S. Cl. 424/94.5; 435/320.1; 435/6.18; 435/6.12; 435/367; 536/23.2; 514/44 R; 435/194

(57) ABSTRACT

Use of the large form of human 2′,5′-Oligoadenylate Synthetase (OAS3) for diagnosis, prevention and treatment of infection with positive-sense single-stranded RNA viruses and for prediction of human genetic susceptibility to positive-sense single-stranded RNA virus-related diseases.

The invention relates to the large form of human 2′,5′-OligoAdenylate Synthetase (OAS3) as a medicament for preventing infection with or treating positive-sense single-stranded RNA viruses.

The invention relates also the large form of human 2′,5′-OligoAdenylate Synthetase (OAS3) as a marker for determining the genetic susceptibility to infection with positive-sense single-stranded RNA viruses.

Flaviviridae and Togaviridae are two positive-sense single-stranded (ss)RNA virus families comprising pathogens that can affect human and animal health world wide. Examples of these viruses include members of dengue, yellow fever (YF), Japanese encephalitis (JE) and tick-borne encephalitis (TBE) antigenic complexes of the Flavivirus genus (Flaviviridae family), members of Eastern Equine Encephalitis (EEE)/Venezuelan Equine Encephalitis (VEE), Semliki Forest (SF), and Sindbis (SIN) groups of Alphavirus genus (Toagaviridae family), and members of Hepacivirus genus (Flaviviridae family) such as Hepatitis C(HCV) and Hepatitis G (HGV) viruses.

Dengue virus, (DV; DEN antigenic complex of flavivirus genus) is endemic in most urban centers of the tropics since a dramatic increase in urbanization created ideal conditions for increased transmission of mosquito-borne dengue disease. The four serotypes of dengue virus (DV-1 to DV-4) are transmitted to humans by the mosquito vector Ae. aegypti. DV infection results in a spectrum of illnesses, ranging form a flu-like disease (dengue fever, DF) to dengue hemorrhagic fever (DHF) that can progress to dengue shock syndrome (DSS) and death. To date, dengue illness is the most important arbovirosis in humans with an estimated 100 millions cases and over 500,000 cases of DHF/DSS occurring each year, including about 25,000 fatal cases, mainly in children under the age 15. Epidemics with a high frequency of DHF/DSS continue to expand geographically in Asia and South America. Despite increased health and economic impacts, the pathogenesis of dengue disease is currently poorly understood. There is no available anti-dengue vaccine or specific therapy for the treatment of dengue virus infection.

West Nile virus (WNV; JE antigenic complex of flavivirus genus) circulates in natural transmission cycles involving mosquitoes (Culex species) and birds, and horses and human are incidental hosts. Zoonotic WNV became a major health concern in North America, the Middle East, and Europe, due to the emergence of a highly neuroinvasive strain in Israel in 1998 (variant Isr98/NY99 from clade Ia of the WN virus lineage I) (Ceccaldi et al., FEMS Microbiol. Lett., 2004, 233, 1-6). WNV infects the central nervous system and causes viral encephalitis in a large range of animal species. In Humans, clinical infections can range in severity from uncomplicated West Nile fever to fatal meningo-encephalitis. The emergence of WNV has been associated with a dramatic increase in the severity of infection in humans drawing the attention to viral encephalitis as a public health concern. Within the last 10 years, WNV has spread across the Western Hemisphere and the Caribbeans. The US outbreaks of WNV have involved thousands of patients causing severe neurological diseases (meningoencephalitis and poliomyelitis-like syndrome) and hundreds of associated deaths. Although mosquito-borne transmission of WNV is the predominant mode, WNV infection transmitted by blood transfusion, organ donation and transplacental transmission to the foetus were also recognized. There is no available vaccine or anti-viral therapy for WNV-related disease.

Chikungunya virus (CHIKV; SF group of alphavirus) is widespread throughout Africa, Southeast Asia, India and Western Pacific, and numerous epidemics have been reported in these areas. Clinically, infection by CHIKV results in fever, rash and intense, invalidating and sometimes persistent arthralgia. In 2005-06, CHIK virus has spread to the south of the Indian Ocean, particularly on La Réunion Island (France), where the outbreak has involved hundreds of thousand of patients. More recently the virus emerged in the east of Italy, where more than 200 people were infected.

Hepatitis C virus (HCV) infection is common worldwide; it is estimated that about 3% of the world's population have HCV and there are about 4 million carriers in Europe alone. Between 20 and 30% of these individuals will develop hepatic cirrhosis and its long-term sequelae such as hepatocellular carcinoma. Treatment of HCV infection with pegylated interferon alpha (IFN-α) in combination with ribavirin may achieve a sustained response in patients infected with HCV viral genotypes 2 or 3 (80%) but the response rate is much lower in patients infected with HCV viral genotype 1 (42%).

Innate antiviral mechanisms mediated by Type-I interferons (IFN-α/β) are potentially the most important pathways of host cell defense limiting viral replication. Indeed, IFN-α/13 are able to trigger the activation of a specific signal transduction pathway leading to the induction of IFN-stimulated genes (ISGs) that are responsible for the establishment of an antiviral state. The ISGs believed to affect RNA virus replication in single cells are the RNA-specific Adenosine Desaminase (ADAR), the proteins of the myxovirus resistance (Mx) family, the double-stranded RNA-dependent protein kinase (PIER), and the 2′,5′-oligoadenylate synthetase (2′,5′-OAS or OAS) family associated to endoribonuclease RNase L.

The OAS/RNase L system is a RNA decay pathway known to play an important role in the established endogeneous antiviral pathway. Binding of enzymatically active OAS to activator double-stranded (ds) viral RNA results in the production of 2′- to 5′-linked oligoadenylates (2-5A). Latent monomeric RNase L is enzymatically activated through homodimerization induced by binding to 2-5A oligomers. Once activated RNase L degrades single-stranded RNA molecules including mRNA and viral RNA, suppressing viral replication (Silverman, J. Virol. 81: 12720, 2007).

Human OAS is a family of enzymes encoded by three closely linked genes on chromosome 12q24.2, with the following order: small (OAS1, p40/46), medium (OAS2, p69/71), and large (OAS3, p100) OAS isoforms (Hovnanian et al., Genomics, 1998, 52, 267-277; Rebouillat, D. and Hovanessian, A. G., Journal of Interferon and Cytokine Research, 1999, 19, 295-308; Rebouillat et al., Genomics, 2000, 70, 232-240; Justesen et al., Cellular and Molecular Life Sciences, 2000, 57, 1593-1612; Rebouillat; Hovanessian, A. G., Cytokine and Growth Factor Reviews, 2007, 18, 351-361). Each OAS gene consists of a conserved OAS unit composed of five translated exons (exons A-E). OAS1 has one unit, whereas OAS2 and OAS3 have two and three units, respectively, and all three genes encode active 2′,5′-Oligoadenylate Synthetase. Another gene, OASL (OAS-Like) encodes a single-unit of OAS-like protein, which however, lacks 2′-5′ synthetase activity (Hartmann et al., Nucleic Acids Res., 1998, 26, 4121-4128; Rebouillat et al., Eur. J. Biochem., 1998; 257, 319-330). Within each size class, multiple members arise as a result of alternate splicing of the primary transcript. The OAS proteins share a conserved unit/domain of about 350 amino acids (OAS unit); OAS1 (p40/p46), OAS2 (p69/71) and OAS3 (p 100) contains one, two and three tandem copies of the OAS unit, respectively. Each OAS protein accumulates in different cellular locations, require different amounts of dsRNA to be activated, and catalyse the formation of differently sized 2-5A products. Whether OAS1 functions as a tetramer, and OAS2 is only active as a dimer, OAS3 has been observed only as a monomer. In addition, the large form of human OAS is presumably not involved in RNase L activation (for review, Rebouillat, D. and Hovanessian, A. G., Journal of Interferon and Cytokine Research, 1999, 19, 295-308).

The first direct evidence for the involvement of OAS family in the antiviral effect exhibited by IFN was provided by transfection of 2′,5′-oligoadenylate synthetase (OAS) cDNA into cells. Overexpression of OAS1 or OAS2 leads to resistance of cells to picornavirus replication (Hovanessian, A. G., Cytokine and Growth Factor Reviews, 2007, 18, 351-361). The importance of OAS1 for clearing WNV infection in vivo was also supported by the finding that murine Oas1b, the orthologous gene of human OAS1, may play a key role into the susceptibility/resistance phenotype of mice to WNV-induced encephalitis (Mashimo, T. et al., Proc. Natl. Acad. Sci. USA, 2002, 99, 11311-11316; Lucas et al., Immunol. Cell. Biol., 2003, 81, 230-236; Kajaste-Rudnitski et al., Journal of Biological Chemistry, 2006, 281, 46244637; International PCT Application WO 02/081741). Analysis of the OAS genetic polymorphism in human demonstrated that genetic markers in OAS genes were the most strongly associated with enzyme activity. Given that OAS1 is an excellent candidate for a human gene that influences host susceptibility to viral infection (Bonnevie-Nielsen et al., Am. J. Hum. Genet., 2005, 76, 623-633), genetic variations in human OAS1 as well as OASL genes were associated to the risk of viral encephalitis, type 1 DM, HCV related disease and other virus infection. With a particular emphasis on HCV disease, a series of OAS1 genotypes linked with the outcome of HCV infection has been reported (International PCT Application WO 03/089003 and WO 2005/040428).

Whether antiviral activity of OAS family is selective for positive-sense ssRNA viruses is a critical issue that it remains to be investigated. It has been reported ectopic expression of OAS1 leads to resistance to encephalomyocarditis virus (picornavirus) replication, but not VSV virus replication. However, OAS is considered to display no role in the elimination of HCV in patients treated with IFN-α, as opposed to other ISGs such as IFN-inducible RNA-dependent protein kinase (PKR) and myxovirus resistance 1 (M×A) genes. Transcription levels of OAS, PKR and Mx were up-regulated after DV infection in non-human primate model (Sariol et al., Clinical and Vaccine Immunology, 14, 2007, 756-766). The analysis of gene transcription pattern associated to Dengue Shock Syndrome showed that OAS3 gene transcripts were less abundant in dengue patients with DSS than in those with non-DSS (Simmons et al J. Infect. Dis., 195:1097, 2007). However, the role of these ISGs in the pathogenesis of DV infection remains poorly understood.

It has been reported that CHIKV is highly sensitive to the antiviral action of type I interferons (IFN-α/β), (Couderc et al., PloS Pathogens, 2007, 4, e29). In the case of alphaviruses, a body of evidence exists to suggest that IFN-mediated inhibition of virus growth does not require RNase L (Ryman et al., J. Virol., 2005, 79, 1487-1499; for review, Silverman, J. Virol., 2007, 81, 12720-9). Whether any members of human ISGs such as OAS family could exert antialphaviral activity is a critical issue that it remained to be investigated.

For the first time, the inventors demonstrate a role for OAS3 in the established endogenous antiviral pathway against positive-sense ssRNA viruses such as alphaviruses (CHIKV, SINV, and SFV). They show that OAS3 acts on the stages of CHIKV growth in blocking viral protein synthesis and viral RNA replication inside the infected human epithelial cells. Very little information is available on the human genetic susceptibility to alphavirus infection. Screening the OAS3 gene for polymorphism in healthly Caucasian individuals identified a single-nucleotide polymorphism (SNP) at the first position of codon CGA-844 where the substitution T for C resulted in a non-sense mutation (OAS3.R844X). The SNP at position OAS3.R844X is expected to result in a truncated form of OAS3 protein, lacking about 20% from the carboxy-terminus. Ectopic expression of mutant OAS3 resulted in a lower efficiency of CHIKV inhibition as compared to full-length OAS3 protein. The notion that genetic polymorphism of OAS3 could control its antialphaviral activity suggests a role of human OAS genes in the pathogenesis of alphavirus-related disease such as Chikungunya fever.

For the first time, the inventors demonstrate that OAS3 exerts antiflaviviral activity in human cells infected with DV (hepatoma cells) and WNV (hepatoma and epithelial cells). Inspection of the complete OAS3 gene in DSS patients and non-DSS dengue patients identified the SNP rs2285993 at the 3^{rd} position of codon AGG-381 where the substitution G to C resulted to amino acid change from Arg to Ser. Our genetic data suggest that variant Ser-381 is associated with a dominant protection against the risk of DSS in dengue That patients. The inventors demonstrate here that OAS3 with Ser-381 is a potent inhibitor of DV growth in human hepatocytes.

The inventors also demonstrate that live-attenuated vaccine strain 17D-204 of YFV (STAMARIL, Sanofi-Pasteur) has inherent resistance to OAS3-mediated antiviral pathway in infected human epithelial and hepatoma cells. However IFN-α was able to establish an antiviral state against vaccine strain 17D-204 of YFV in human cells.

These findings are useful for the development of OAS3-based prophylaxis and therapy against positive-sense ssRNA viruses of major medical importance including CHIKV, WNV and DV. They are also useful for the development of new OAS3-based molecular tools for the prediction of human susceptibility to the infection with alphaviruses, flaviviruses and other positive-sense ssRNA viruses of major medical importance, and in particular for the prediction of severe forms of disease in dengue, West Nile and Chikungunya patients.

A subject of the invention is an isolated 2′,5′-oligoadenylate synthetase 3 protein or an isolated polynucleotide encoding said 2′-5′-oligoadenylate synthetase 3 protein, as a medicament.

### DEFINITIONS

“polynucleotide” refers to a genomic DNA fragment, a cDNA fragment or an RNA molecule.
“2′-5′-oligoadenylate synthetase 3”, “OAS3”, “OAS 3”, “2′-5′-oligoadenylate synthetase 3 (100 kD)”, (2-5′)oligo(A)synthetase 3″, “oligoadenylate synthetase p100”, “p100 OAS” or “p100OAS” refers to a protein which is encoded by the OAS3 gene of a mammal and has 2′-5′-oligoadenylate synthetase activity, and to the derived variants, including natural variants resulting from polymorphism in the OAS3 gene and artificial variants resulting from mutation (insertion, deletion, substitution) of one or more nucleotides in the OAS3 gene/open reading frame (ORF) sequences, providing that the variant has 2′-5′-oligoadenylate synthetase activity and is capable of inhibiting positive-sense single-stranded RNA virus replication. Preferably, the variant comprises three OAS domains. The OAS3 gene and the deduced OAS3 ORF and amino acid sequence of various mammals are available in sequence databases and other OAS3 gene/ORF sequences may be determined by standard cloning and sequencing techniques which are known by one skilled in the art.
“human OAS3 gene” is a 34806 bp sequence corresponding to positions 111860632 to 111895437 on GenBank sequence accession number NC_{—}000012. The human OAS3 gene is located on chromosome 12 (12q24.2) and comprises 16 Exons: Exon 1: 1 to 264; Exon 2: 3127 to 3409; Exon 3: 6033 to 6208; Exon 4: 8300 to 8538; Exon 5: 9503 to 9656; Exon 6: 10418 to 10762; Exon 7: 12250 to 12532; Exon 8: 22628 to 22803; Exon 9: 24209 to 24459; Exon 10: 24870 to 25014; Exon 11: 25792 to 25965; Exon 12: 27301 to 27586; Exon 13: 28975 to 29150; Exon 14: 29493 to 29731; Exon 15: 31165 to 31312; Exon 16: 31519 to 34806.
“human OAS3 open reading frame (ORF)” is the sequence SEQ ID NO: 1 which corresponds to positions 88 to 3351 on the 6646 bp human cDNA sequence GenBank accession number: NM_{—}006187 (SEQ ID NO: 27). The positions of Exons 1 to 16 on the cDNA sequence NM_{—}006187 are: 1 to 264, 265 to 547, 548 to 723, 724 to 962, 963 to 1116, 1117 to 1461, 1462 to 1744, 1745 to 1920, 1921 to 2171, 2172 to 2316, 2317 to 2490, 2491 to 2776, 2777 to 2952, 2953 to 3191, 3192 to 3339, 3340 to 6627, respectively.
“human OAS3 protein” is a 1087 amino acid sequence corresponding to GenBank accession number NP_{—}006178 or SEQ ID NO: 2; the three OAS domains correspond to positions 6 to 343 (SEQ ID NO: 3), 411 to 742 (SEQ ID NO: 4) and 750 to 1084 (SEQ ID NO: 5), respectively.
“OAS3 activity” refers to both 2′-5′-oligoadenylate synthetase and positive-sense single-stranded RNA virus replication inhibition activities.

The 2′,5′-oligoadenylate synthetase activity of the OAS3 protein of the invention may be assayed by chromatographic or electrophoretic methods to determine the end-point amounts of oligoadenylates formed (St Laurent et al., Cell, 1983, 33, 95-102; Johnston et al., In: Interferon 3: Mechanisms of Production and Action, 1984, 189-298, Friedman, R. M., Ed, Elsevier, Amsterdam; Justesen et al., Proc. Natl. Acad. Sci., USA, 1980, 77, 4618-4622; Justesen et al., Nucleic Acids Res., 1980, 8, 3073-3085; Justesen, J. and Kjelgaard, N. O., Anal. Biochem., 1992, 207, 90-93).
“inhibition of positive-sense ssRNA virus replication by the OAS3 protein of the invention” refers to the partial or total reduction of virus growth (virus multiplication) when exogenous OAS3 protein (not encoded by the genome of the cells; recombinant OAS3 protein, for example) is present in the virus-infected cells. This inhibition may be determined by infecting an appropriate recombinant cell line expressing the OAS3 protein with a positive-sense single-stranded RNA virus. Non-recombinant cells of the same type infected with the virus are used as control. Then, progeny virus production in the supernatant of the virus-infected cells may be measured by any well-known virus titration assay. Alternatively, viral proteins production may be analyzed by Western-Blot or Immunolabeling of viral antigens or viral genomic and subgenomic RNAs production may be analyzed by Northern-Blot or RT-PCR.
“positive-sense ssRNA virus” refers to a virus that has positive-sense single-stranded ribonucleic acid (ssRNA) as its genetic material and does not replicate using a DNA intermediate. Positive-sense ssRNA viruses belong to Group IV of the Baltimore classification system of classifying viruses.
“identity” with respect to both amino acid sequences and nucleic acid sequences, refers to a measure of the degree of identity of two sequences based upon alignment of the sequences which maximizes identity between aligned amino acid residues or nucleotides, an which is a function of the number of identical residues or nucleotides, the number of total residues (1087 residues in the case of the present invention) or nucleotides (3261 nucleotides in the case of the present invention), and the presence and length or gaps in the sequence alignment. Various alignment algorithms and/or computer programs are available for determining sequence identity using standard parameters, including FASTA, or BLAST which are available as a part of the GCG sequence analysis package (University of Wisconsin, Madison, Wis.), and can be used with, e.g., default settings.

A protein having 75% identity with residues 1 to 1087 of SEQ ID NO: 2 is a protein whose sequence may include up to (25×10.87=271) changes when the protein sequence is aligned and compared to residues 1 to 1087 of with SEQ ID NO: 2. One change refers to the deletion, substitution or insertion of one amino acid as compared to residues 1 to 1087 of SEQ ID NO: 2. For example, a 1080 amino acid sequences having 50 changes with the first 1080 residues of SEQ ID NO: 2 has 1080−50/10.87=94.75% identity with SEQ ID NO: 2. For example, a 1090 amino acid sequences having 50 changes with residues 1 to 1087 of SEQ ID NO: 2 has 1087−50/10.87=95.4% identity with SEQ ID NO: 2.
“similarity” refers to a measure of the degree of similarity of two amino acid sequences based upon alignment of the sequences which maximizes similarity between aligned amino acid residues, and which is a function of the number of identical or similar residues, the number of total residues (1087 residues in the case of the present invention), and the presence and length or gaps in the sequence alignment. Various alignment algorithms and/or computer programs are available for determining sequence similarity using standard parameters, including FASTA, or BLAST which are available as a part of the GCG sequence analysis package (University of Wisconsin, Madison, Wis.), and can be used with, e.g., default settings. Similar residues refer to residues having comparable chemical properties (size, charge (neutral, basic, acidic), hydrophilicity/hydrophobicity).
“individual” includes mammals, as well as other vertebrates (e.g., birds, fish and reptiles). The terms “mammal” and “mammalian”, as used herein, refer to any vertebrate animal, including monotremes, marsupials and placental, that suckle their young and either give birth to living young (eutharian or placental mammals) or are egg-laying (metatharian or nonplacental mammals). Examples of mammalian species include humans and other primates (e.g., monkeys, chimpanzees), rodents (e.g., rats, mice, guinea pigs) and others such as for example: cows, pigs and horses.
by “mutation” is intended the substitution, deletion, insertion of one or more nucleotides/amino acids in a polynucleotide (cDNA, gene) or a poly-peptide sequence. Said mutation can affect the coding sequence of a gene or its regulatory sequence. It may also affect the structure of the genomic sequence or the structure/stability of the encoded mRNA.

The invention encompasses modified OAS3 protein including one or more modifications selected from the group consisting of: the mutation (insertion, deletion, substitution) of one or more amino acids in the OAS3 amino acid sequence, the addition of an amino acid fusion moiety, the substitution of amino acid residues by non-natural amino acids (D-amino-acids or non-amino acid analogs), the modification of the peptide bond, the cyclization, the addition of chemical groups to the side chains (lipids, oligo- or -polysaccharides), and the coupling to an appropriate carrier. These modifications which are introduced by procedures well-known in the art, result in a modified OAS3 protein which is still active for 2′-5′-oligoadenylate synthetase and inhibition of positive-sense single-stranded RNA virus replication activities.

According to a preferred embodiment of the invention, said 2′-5′-aligoadenylate synthetase 3 (OAS3) is human 2′-5′-oligoadenylate synthetase 3.

According to another preferred embodiment of the invention, said OAS3 protein has at least 70% amino acid sequence identity or 80% amino acid sequence similarity, preferably at least 80% amino acid sequence identity or 90% amino acid sequence similarity to residues 1 to 1087 of SEQ ID NO: 2.

According to a more preferred embodiment of the invention, said OAS3 protein comprises a Serine at position 381 of SEQ ID NO: 2. Preferably, said OAS3 protein comprises or consists of an amino acid sequence selected in the group consisting of: SEQ ID NO: 2 and SEQ ID NO: 7.

According to another preferred embodiment of the invention, said OAS3 polynucleotide is coding for a protein as defined above, more preferably it comprises or consists of a nucleotide sequence selected in the group consisting of: SEQ ID NO: 1 which encodes the protein SEQ ID NO: 2 and SEQ ID NO: 6 which encodes the protein SEQ ID NO: 7.

According to another preferred embodiment of the invention, said polynucleotide is inserted in an expression vector.

A vector refers to a nucleic acid molecule capable of transporting another nucleic acid to which it has been linked. A vector which can be used in the present invention includes, but is not limited to, a viral vector, a plasmid, a RNA vector or a linear or circular DNA or RNA molecule which may consists of a chromosomal, non chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available.

Viral vectors include retrovirus, adenovirus, parvovirus (e.g. adeno-associated viruses or AAVs), coronavirus, negative strand RNA viruses such as ortho-myxovirus (e.g., influenza virus), rhabdovirus (e.g., rabies and vesicular stomatitis virus), paramyxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).

Preferably said vectors are expression vectors, wherein the sequence encoding the OAS3 protein of the invention is placed under control of appropriate transcriptional and translational control elements to permit production or synthesis of said protein. Therefore, said polynucleotide is comprised in an expression cassette. More particularly, the vector comprises a replication origin, a promoter operatively linked to said encoding polynucleotide, a ribosome-binding site, an RNA-splicing site (when genomic DNA is used), a polyadenylation site and a transcription termination site. It also can comprise an enhancer. Selection of the promoter will depend upon the cell in which the polypeptide is expressed. Suitable promoters include tissue specific and/or inducible promoters. Examples of inducible promoters are: eukaryotic metallothionine promoter which is induced by increased levels of heavy metals, heat shock promoter which is induced by increased temperature. Examples of tissue specific promoters are skeletal muscle creatine kinase, prostate-specific antigen (PSA), α-antitrypsin protease, human surfactant (SP) A and B proteins and β-casein. Vectors can comprise selectable markers, for example: neomycin phosphotransferase, histidinol dehydrogenase, dihydrofolate reductase, hygromycin phosphotransferase, herpes simplex virus thymidine kinase, adenosine deaminase, glutamine synthetase, and hypoxanthine-guanine phosphoribosyl transferase for eukaryotic cell culture; TRP1, URA3 and LEU2 for S. cerevisiae; tetracycline, rifampicin or ampicillin resistance in E. coli.

The choice of the vector depends on their use (stable or transient expression) or and on the host cell; viral vectors and “naked” nucleic acid vectors are preferred vectors for expression in mammal cells (human and animal). Use may be made, inter alia, of viral vectors such as adenoviruses, retroviruses, lentiviruses and AAVs, into which the sequence of interest has been inserted beforehand.

The subject-matter of the present invention is also a pharmaceutical composition characterized in that it comprises at least one OAS3 protein or one OAS3 polynucleotide, preferably inserted in an expression vector, as defined above, and at least one acceptable vehicle, carrier, additive and/or immunostimulating agent.

Any suitable carrier known to those of ordinary skill in the art may be employed in the pharmaceutical composition of the present invention, the type of carrier varying depending on the mode of administration. For parenteral administration, such as subcutaneous injection, the carrier preferably comprises water, saline buffer, lactose, mannitol, glutamate, a fat or a wax and the injectable pharmaceutical composition is preferably an isotonic solution (around 300-320 mosmoles). For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g. polylactic galactide) may also be employed as carriers for the pharmaceutical compositions of this invention. Suitable biodegradable microspheres are disclosed, for example in U.S. Pat. Nos. 4,897,268 and 5,075,109. The additive may be chosen among antiaggregating agents, antioxidants, dyes, flavor enhancers, or smoothing, assembling or isolating agents, and in general among any excipient conventionally used in the pharmaceutical industry. Any of the variety of immunostimulating agent may be employed in the compositions of the present invention to enhance the immune response.

The pharmaceutical composition may be in a form suitable for oral administration. For example, the composition is in the form of tablets, ordinary capsules, gelatine capsules or syrup for oral administration. These gelatine capsules, ordinary capsules and tablet forms can contain excipients conventionally used in pharmaceutical formulation, such as adjuvants or binders like starches, gums and gelatine, adjuvants like calcium phosphate, disintegrating agents like cornstarch or algenic acids, a lubricant like magnesium stearate, sweeteners or flavourings. Solutions or suspensions can be prepared in aqueous or non-aqueous media by the addition of pharmacologically compatible solvents. These include glycols, polyglycols, propylene glycols, polyglycol ether, DMSO and ethanol.

The OAS3 protein or the OAS3 polynucleotide (isolated or inserted in a vector) are introduced into cells, in vitro, ex vivo or in vivo, by any convenient means well-known to those in the art, which are appropriate for the particular cell type, alone or in association with at least either an appropriate vehicle and/or carrier. For example, the OAS3 protein/polynucleotide may be associated with a substance capable of providing protection for said sequences in the organism or allowing it to cross the host-cell membrane. The OAS3 protein may be advantageously associated with liposomes, polyethyleneimine (PEI), and/or membrane translocating peptides (Bonetta, The Scientist, 2002, 16, 38; Ford et al., Gene Ther., 2001, 8, 1-4; Wadia and Dowdy, Curr. Opin. Biotechnol., 2002, 13, 52-56; Langel, U. In Handbook of cell penetrating peptides (2nd Ed.), 2006, Lavoisier, FRANCE); in the latter case, the sequence of the OAS3 protein is fused with the sequence of a membrane translocating peptide (fusion protein). Polynucleotide encoding OAS3 (isolated or inserted in a vector) may be introduced into a cell by a variety of methods (e.g., injection, direct uptake, projectile bombardment, liposomes, electroporation). OAS3 protein can be stably or transiently expressed into cells using appropriate expression vectors as defined above.

In one embodiment of the present invention, the OAS3 protein/polynucleotide is substantially non-immunogenic, i.e., engenders little or no adverse immunological response. A variety of methods for ameliorating or eliminating deleterious immunological reactions of this sort can be used in accordance with the invention. In a preferred embodiment, the OAS3 protein is substantially free of N-formyl methionine. Another way to avoid unwanted immunological reactions is to conjugate protein/polynucleotide to polyethylene glycol (“PEG”) or polypropylene glycol (“PPG”) (preferably of 500 to 20,000 daltons average molecular weight (MW)).

Another subject of the present invention is an OAS3 protein or a polynucleotide coding for said OAS3 protein as defined above for preventing or treating an infection with a positive-sense single-stranded RNA virus.

According to a more preferred embodiment, said virus is of the Alphavirus genus. More preferably, it is selected from the group consisting of: Chikungunya (CHIK), Sindbis (SIN), Semliki Forest (SF), Eastern Equine Encephalitis (EEE), Western Equine Encephalitis (WEE), Venezuelan Equine Encephalitis (VEE), Ross River (RR), O'Nyong Nyong (ONN) and Banna Forest (BF) viruses.

According to another more preferred embodiment, said virus is of the Flavivirus genus. More preferably, said virus is selected from the group consisting of: Dengue, Japanese Encephalitis, Kyasanur Forest Disease, Murray Valley Encephalitis, St. Louis Encephalitis, Tick-Borne Encephalitis, West Nile, Yellow Fever and Omsk hemorrhagic fever (OHF) virus.

According to another more preferred embodiment, said virus is of the Hepacivirus genus. More preferably, said virus is the Hepatitis C virus.

The subject-matter of the present invention is also products containing at least an OAS3 protein or an OAS3 polynucleotide, preferably inserted in an expression vector, as defined above and a second product which is different from the first one, said second product being selected from the group consisting of: antiviral, anti-inflammatory and immunomodulatory drugs, as a combined preparation for simultaneous, separate or sequential use in the prevention or the treatment of a positive-sense single-stranded RNA virus infection.

The subject-matter of the present invention is also a method for preventing or curing a positive-sense single-stranded RNA virus infection in an individual in need thereof, said method comprising the step of administering to said individual a composition as defined above, by any means.

In general, the composition may be administered by parenteral injection (e.g., intradermal, intramuscular, intravenous or subcutaneous), intranasally (e.g. by aspiration or nebulization), orally, sublingually, or topically, through the skin or through the rectum.

The amount of OAS3 (protein/polypeptide) present in the composition of the present invention is a therapeutically effective amount. A therapeutically effective amount of OAS3 (protein/polypeptide) is that amount necessary so that OAS3 protein performs its role of inhibiting positive-sense single-stranded RNA virus replication without causing, overly negative effects in the subject to which the composition is administered. The exact amount of OAS3 (protein/polypeptide) to be used and the composition to be administered will vary according to factors such as the positive-sense single-stranded RNA virus species and the individual species (human, animal) being treated, the mode of administration, the frequency of administration as well as the other ingredients in the composition.

Preferably, the composition is composed of from about 10 μg to about 10 mg and more preferably from about 100 μg to about 1 mg, of OAS3 (protein/polypeptide). By “about”, it is meant that the value of said quantity Gig or mg) of OAS3 can vary within a certain range depending on the margin of error of the method used to evaluate such quantity.

For instance, during an oral administration of the composition of the invention, individual to be treated could be subjected to a 1 dose schedule of from about 10 μg to about 10 mg of OAS3 (protein/polypeptide) per day during 3 consecutive days. The treatment may be repeated once one week later.

For parenteral administration, such as subcutaneous injection, the individual to be treated could be subjected to a 1 dose of from about 10 μg to about 10 mg and more preferably from about 100 μg to about 1 mg, of OAS3 (protein/polypeptide). The treatment may be repeated once one week later.

A subject of the invention is also a method in vitro for evaluating the susceptibility of an individual to an infection with a positive-sense single-stranded RNA virus as defined above, comprising: the detection of a polymorphism in the OAS3 gene in a nucleic acid sample obtained from said individual.

The nucleic acid sample may be genomic DNA, total mRNA or cDNA.

The polymorphism is detected by any method known in the art that allows the detection of mutation in nucleic acid sequences as those described for example In Current Protocols in Human Genetics, 2008, John Wiley & Sons, Inc. Examples of genotyping assays include with no limitation: RAPD, RFLP, AFLP, sequence specific oligonucleotide hybridization, SnapShot PCR, Ligase detection reaction, PCR and Maldi-TOF, Pyrosequencing. This assay may use the OAS3 specific primers of Table II and III and in particular the primers SEQ ID NO: 11, 12, 62 to 86 and 118 to 142.

According to a preferred embodiment of said method, said positive-sense single-stranded RNA virus is an alphavirus such as Chikungunya virus or a flavivirus such as Dengue virus.

According to another preferred embodiment of said method, said polymorphism is the mutation of the Arg844 codon to a stop codon (R844X); said polymorphism detected in the Caucasian population, is associated with an increased susceptibility to positive-sense single-stranded RNA virus infection, particularly Chikungunya virus infection. The R844X mutation may be detected by PCR-RFLP using the pair of primers (SEQ ID NO: 11 and SEQ ID NO: 12), followed by digestion of the 183 bp PCR product with BglII; the presence of two fragments of 115 bp and 68 bp indicates the presence of OAS3.R844X mutation.

According to another preferred embodiment of said method, said polymorphism is a single nucleotide polymorphism (SNP) at the third position of codon 381. Preferably, said SNP is a G to C substitution of codon AGG-381 that changes amino acid from Arg to Ser (R381S). Genetic data suggest that variant Ser-381 is associated with a dominant protection against the risk of DSS in dengue patients (Table VI). This SNP may be detected by any appropriate genotyping assay as defined above. For example, this assay may comprise the direct sequencing of genomic DNA amplified with a pair of OAS3 specific primers such as the pair of PCR primers specific to OAS3 exon 6 that is presented in Table III (SEQ ID NO: 70 and 126).

A subject of the invention is also an isolated OAS3 protein comprising or consisting of the sequence SEQ ID NO: 7 or SEQ ID NO: 30.

A subject of the invention is also an isolated OAS3 protein fragment comprising or consisting of the sequence SEQ ID NO: 9; this OAS3 fragment which includes residues 1 to 843 of SEQ ID NO: 7 comprises the first and the second OAS domains of OAS3 but lacks most of the third (C-terminal) OAS domain.

A subject of the invention is also:
an isolated polynucleotide comprising or consisting of the sequence SEQ ID NO: 6, which encodes the OAS3 protein of SEQ ID NO: 7,
an isolated polynucleotide comprising or consisting of the sequence SEQ ID NO: 8, which encodes the OAS3 protein fragment of SEQ ID NO: 9,
an isolated polynucleotide comprising or consisting of the sequence SEQ ID NO: 28 or 29, which encodes the OAS3 protein variant SEQ ID NO: 30.

A subject of the invention is also a recombinant vector, preferably an expression vector comprising a polynucleotide having a sequence selected in the group consisting of: SEQ ID NO: 6, SEQ ID NO: 8, SEQ ID NO: 28 and SEQ ID NO: 29.

A subject of the invention is also a host cell transfected or transformed by a polynucleotide comprising or consisting of a sequence selected in the group consisting of: SEQ ID NO: 6, SEQ ID NO: 8, SEQ ID NO: 28 and SEQ ID NO: 29.

According to a preferred embodiment of the invention, it is an HeLa-Tet-Off cell line expressing a recombinant human OAS3, named HeLa-Tet-Off/OAS3#C417-1, deposited Feb. 26, 2008, at the Collection Nationale de Cultures de Microorganismes, 25 rue du Docteur Roux, 75724 Paris Cedex 15, under the accession number I-3927.

According to another preferred embodiment of the invention, it is an a-Tet-Off cell line expressing a truncated recombinant human OAS3, named HeLa-Tet-Off/OAS3/delta/1C, deposited Apr. 17, 2008, at the Collection Nationale de Cultures de Microorganismes, 25 rue du Docteur Roux, 75724 Paris Cedex 15, under the accession number I-3968.

According to a preferred embodiment of the invention, it is an HepG2-Tet-Off cell line expressing a recombinant human OAS3, named HeLa-Tet-Off/OAS3#F8, deposited May 15, 2009, at the Collection Nationale de Cultures de Microorganismes, 25 rue du Docteur Roux, 75724 Paris Cedex 15, under the accession number I-4158.

These cell lines which are derived from HeLa or HepG2 cells which can be infected with various viruses are useful for assaying the susceptibility of a virus to OAS3 mediated antiviral activity.

A subject of the invention is also a non-human transgenic animal comprising a polynucleotide as defined above.

A subject of the invention is also a transgenic plant comprising a polynucleotide as defined above.

The OAS3 protein/polynucleotide of the invention are prepared using well-known recombinant DNA and genetic engineering techniques. For example, a sequence comprising the OAS3 ORF is amplified from a DNA template, by polymerase chain reaction with specific primers. The PCR fragment is then cloned in an expression vector by using appropriate restriction sites. The OAS3 protein is expressed in a host cell or a transgenic animal/plant modified by the expression vector, under conditions suitable for the expression of the OAS3 protein, and the OAS3 protein is recovered from the host cell culture or from the transgenic animal/plant.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, N.Y.: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986). Current Protocols in Human Genetics (John Wiley & Sons, Inc, 2008), specifically Chapter 12 “Vectors For Gene Therapy” and Chapter 13 “Delivery Systems for Gene Therapy”).

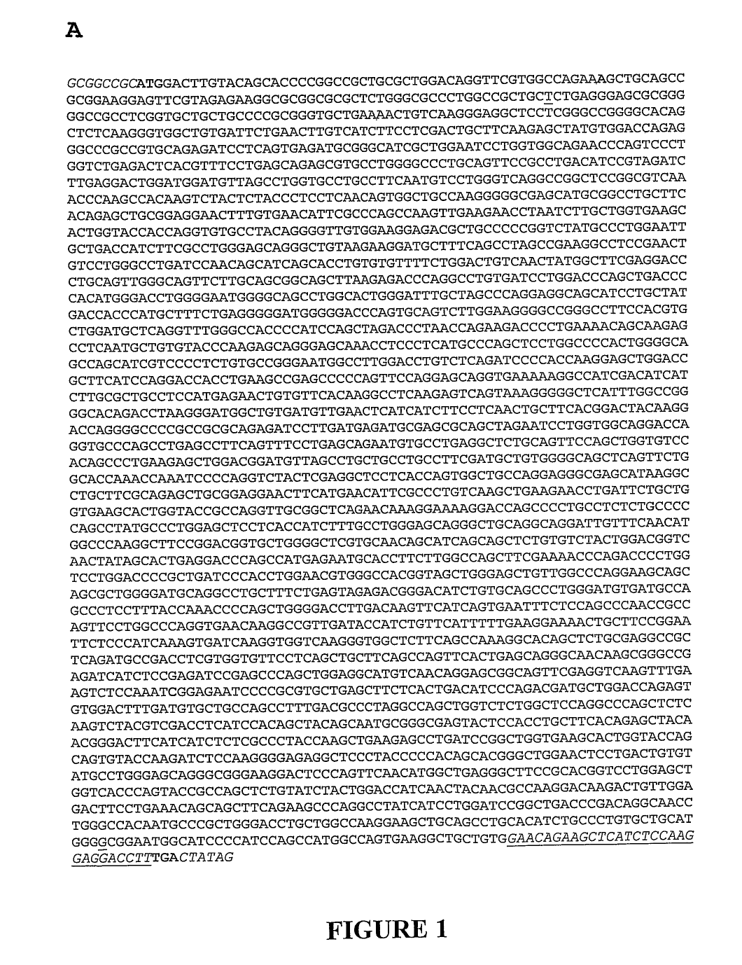

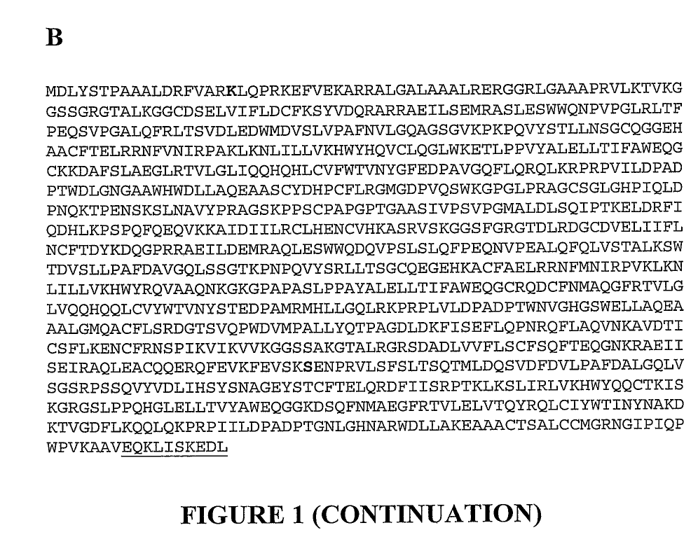

FIG. 1 represents the nucleotide (A) and amino acid (B) sequences of human recombinant OAS3 (clone OAS3C 17.1) tagged with a c-myc epitope. These nucleotide and amino acid sequences correspond to SEQ ID NO: 23 and 24, respectively. Changes between OAS3 mRNA reference sequence (SEQ ID NO: 27) and cloned OAS3 cDNA are underlined if synonymous or shown in bold if non synonymous. The translation initiation and stop codons are in bold. The additional nucleotide and amino acid sequences corresponding to the C_terminal c-myc epitope are underlined. The 5′ NotI and 3′ EcoRV restriction sites are shown in italics.

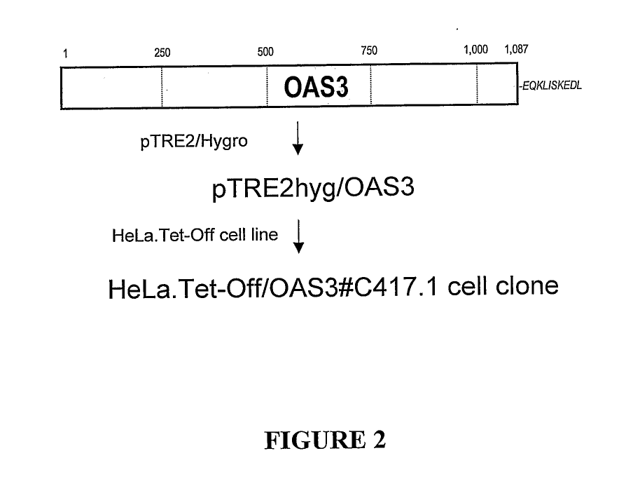

FIG. 2 illustrates the establishment of an inducible HeLa-Tet-Off/OAS3#C417-1 cell line expressing recombinant human OAS3.

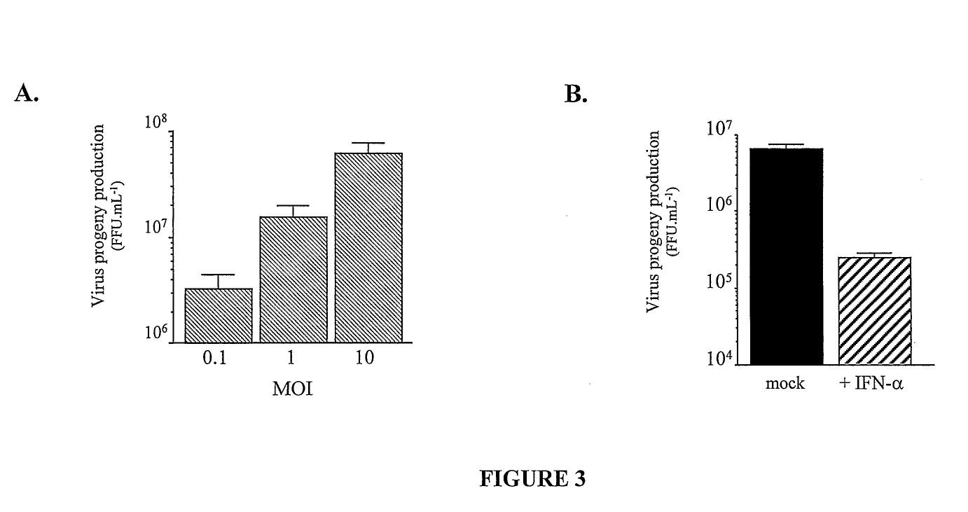

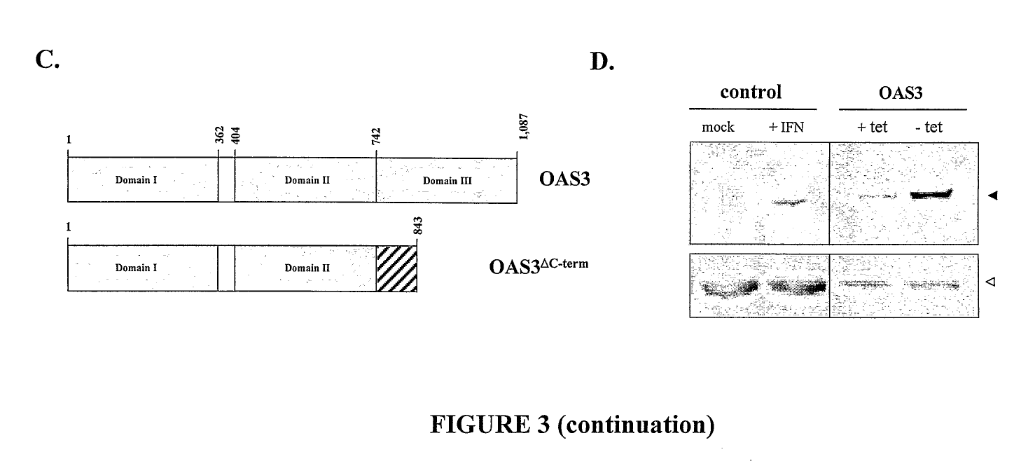

FIG. 3 illustrates the detection of OAS3 in HeLa cells. In (A), HeLa.Tet-Off cells were infected with different Focus Forming Units (AP61FFU) per cell of CHIK virus strain 06-49 (CHIKV 06-49). At 20 h post-infection (p.i.), virus particles produced in the supernatants of infected cells were titered on mosquito Aedes pseudoscutellaris AP61 cells by focus immunoassay. In (B), HeLa.Tet-Off cells were treated with 1,000 IU/ml human IFN-α or mock-treated (control) 5 hours prior to CHIKV input at a multiplicity of infection of one AP61FFU per cell (1 MOI). Virus progeny productions were determined at 18 h p.i as described above. In (C), the graph depicts the structures of full-length and truncated form of OAS3. In (D), expression of ectopic OAS3 in induced (−Tet) or uninduced (+Tet) Tet-Off/OAS3 cells was analyzed by immunoblot analysis using OAS3-specific antibody As controls, HeLa.Tet-Off cells were treated with 1,000 IU IFN-α (+IFN) or mock-treated (mock). The β-actin served as a house-keeping protein control.

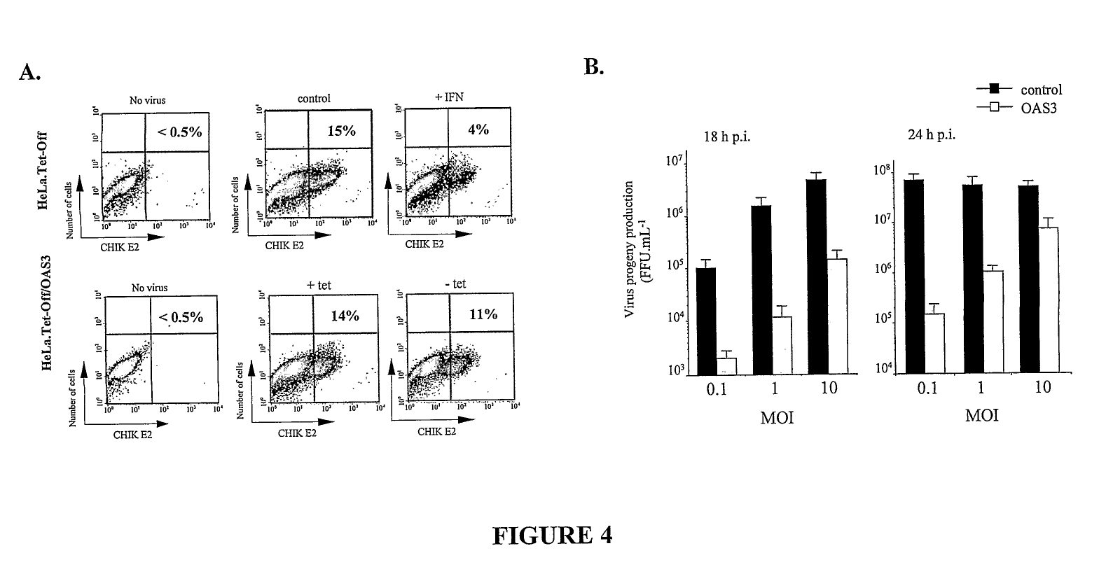

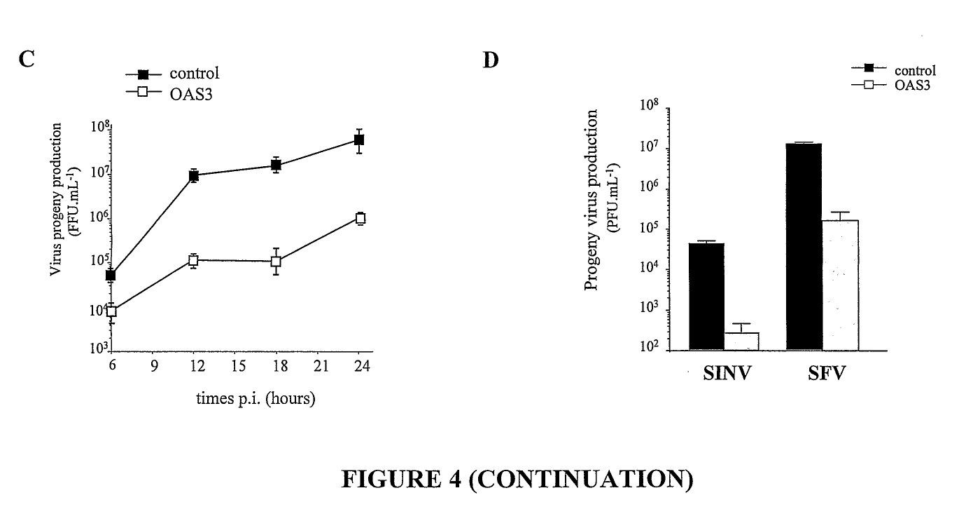

FIG. 4 illustrates the inhibition of CHIKV growth in OAS3-expressing HeLa cells. In (A), cells were infected 18 h with CHIKV at 1 MOI and analyzed by flow cytometry using anti-CHIK.E2 MAb 3E4. Analysis of CHIKV E2 protein production in mock-infected cells (No virus), in CHIKV-infected HeLa.Tet-off cells incubated with 1,000 IU/ml human IFN-α (+IFN) 5 h prior virus input or mock-treated (control), and CHIKV-infected Tet-Off/OAS3 cells in the presence (+tet) or in absence (−tet) of tetracycline. In (B), cells were infected with CHIKV at different MOI and virus progeny productions were determined at 18 and 24 h p.i. In (C), inhibition of CHIKV growth was monitored at various times p.i. In (D), cells were infected with SINV or SFV at 1 MOI and virus progeny productions were determined at 18 h p.i.

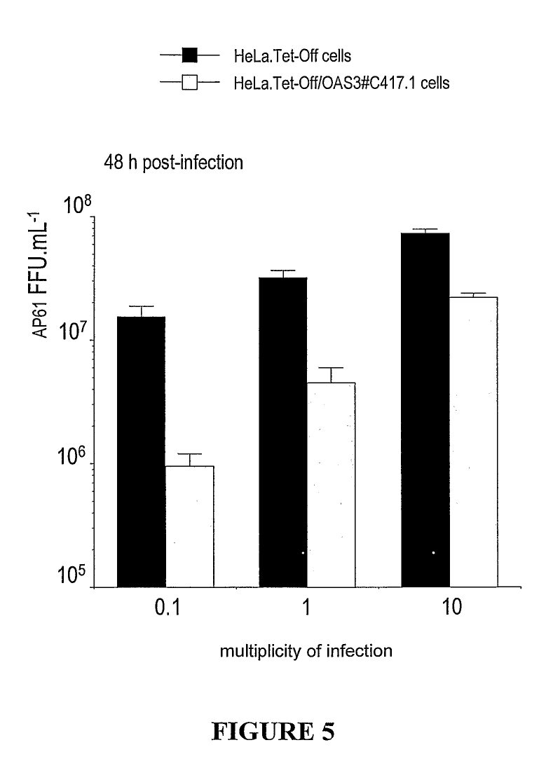

FIG. 5 illustrates WN virus sensitivity to antiviral activities of OAS3. HeLa/Tet-off and induced HeLa.Tet-Off/OAS3#C417-1 cells were infected with 0.1, 1.0 or 10 AP61FFU per cell of WN virus strain IS-98-ST1. Infectious virus particles produced in supernatants of infected cells were titered at 48 hours post-infection by focus immunoassay on mosquito Aedes pseudoscutellaris AP61 cells.

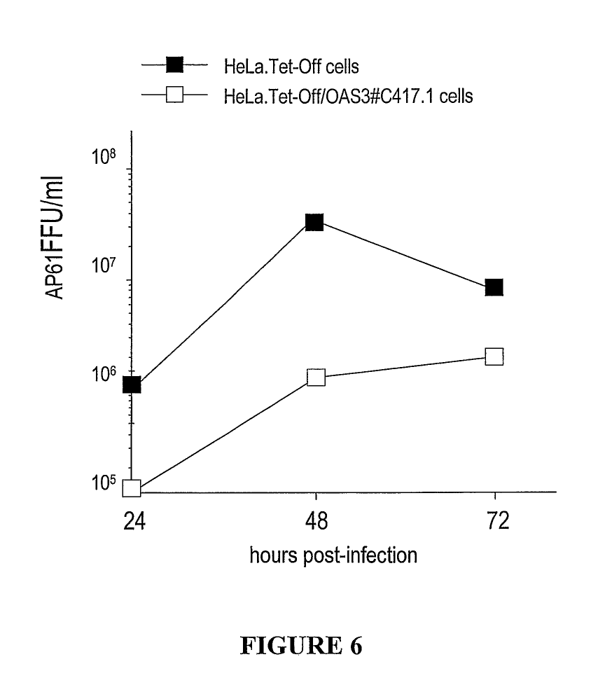

FIG. 6 illustrates inhibition of WN virus growth in OAS3-expressing cells. HeLa/Tet-Off and induced HeLa.Tet-Off/OAS3#C417-1 cells were infected with 0.1 AP61FFU per cell of WN virus strain IS-98-ST1 and infectious virus particles produced in supernatants of infected cells were titered at various times post-infection (24, 48 and 72 hours) by focus immunoassay on mosquito Aedes pseudoscutellaris AP61 cells.

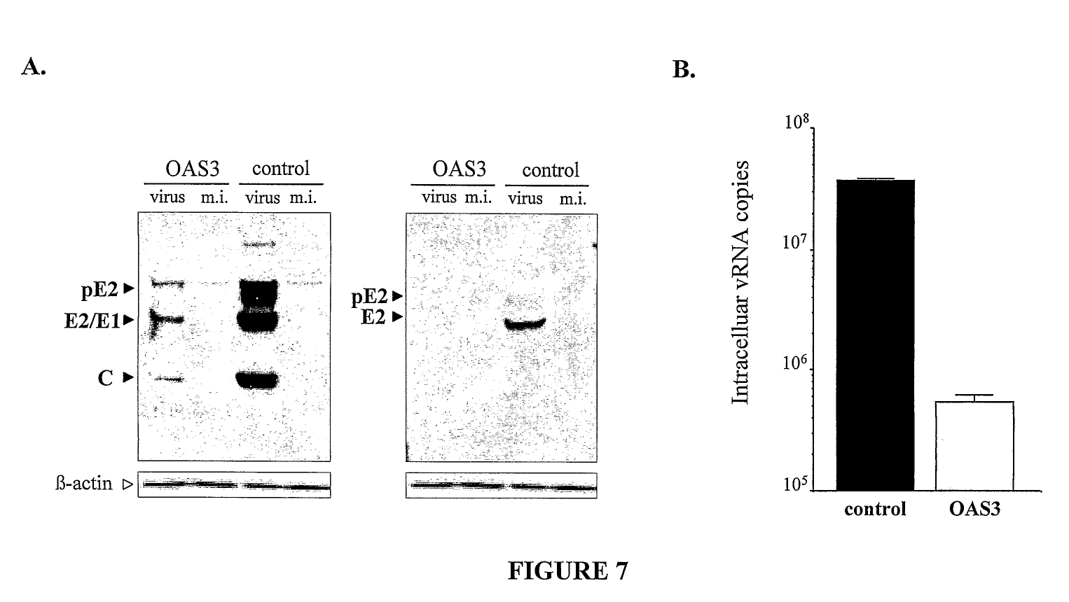

FIG. 7 illustrates the consequences of OAS3 expression for CHIKV replication. In (A), immunoblot assay was performed on cell extracts from HeLa.Tet-off cells (control) and HeLa.Tet-Off/OAS3 (OAS3) cells infected with CHIKV (virus) or mock-infected (m.i.) using anti-CHIK HMAF (left) or anti-CHIKV E2 MAb 3E4 (right). The β-actin served as protein control. In (B), real-time RT-PCR analysis of viral RNA production in HeLa.Tet-Off cells (control) and HeLa.Tet-Off/OAS3 cells (OAS3) infected with CHIKV at 1 MOI was performed at 8 h p.i.

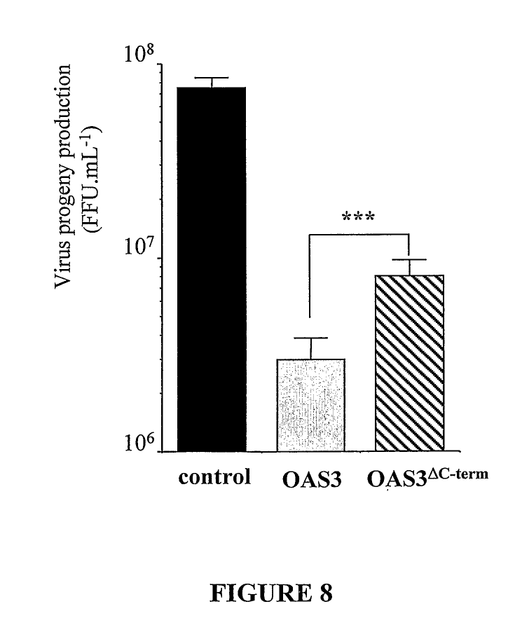

FIG. 8 illustrates the anti-alphaviral activity of truncated form of OAS3. HeLa.Tet-off (control), HeLa.Tet-Off/OAS3 (OAS3) and HeLa.Tet-Off/OAS3/delta/1C(OAS3^{ΔC-term}) cells were infected with CHIKV at 1 MOI and virus progeny productions were determined at 18 h p.i. The values were compared statistically according to Student's t tests (***: P<0.001).

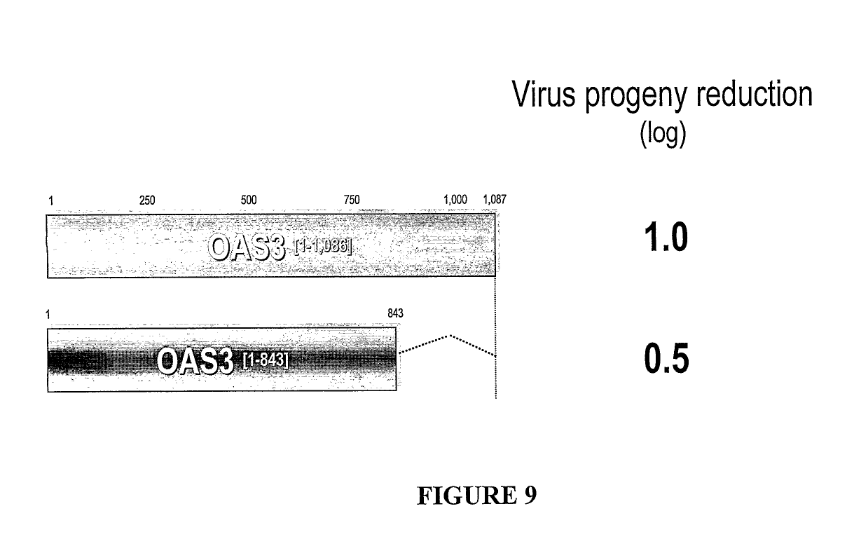

FIG. 9 illustrates the anti-flaviviral activity of truncated form of OAS3. HeLa.Tet-off (control), HeLa.Tet-Off/OAS3 (OAS3) and HeLa.Tet-Off/OAS3/delta/1C (OAS3^{ΔC-term}) cells were infected with WNV at 1 MOI and virus progeny productions were determined at 18 h p.i. The values were compared statistically according to Student's t tests (***: P<0.001).

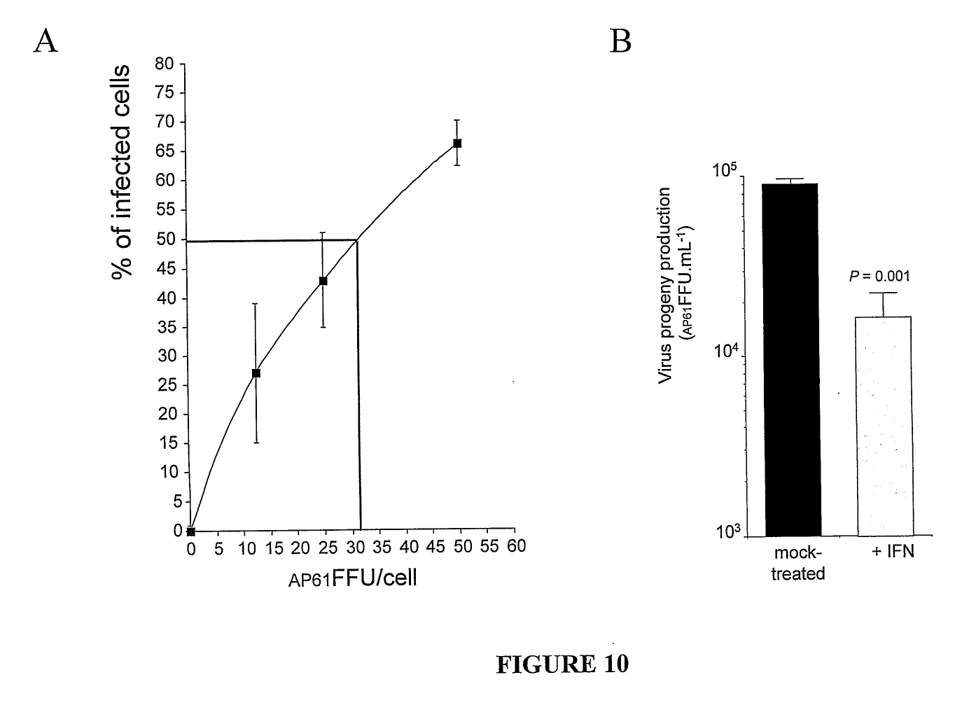

FIG. 10 illustrates DV-1 virus sensitivity to Type-I IFN pathway. A: Susceptibility of HepG2.Tet-Off cell line to DV infection. Cells were infected with increasing input (AP61FFU/per cell) of DV-1 virus strain FGA/NA d1d. At 40 h post-infection, the cells were analyzed by flow cytometry using MAb 4E11 reactive to DV E glycoprotein. B: DV-1 virus sensitivity to Type-I IFN pathway. HepG2.Tet-off cells were pretreated with 1,000 IU·mL⁻¹ human IFN-α (+IFN) or not treated (mock-treated) 5 hours prior DV infection (10 MOI). At 40 h p.i., virus progeny production was determined as previously described (Duarte dos Santos et al., Virology, 2000, 274, 292-).

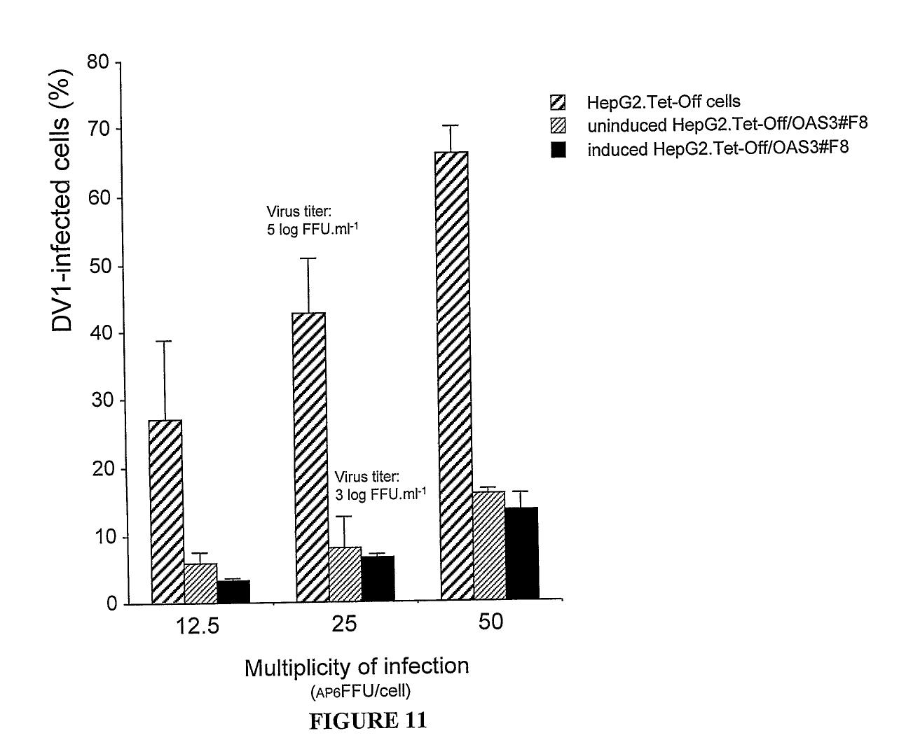

FIG. 11: Inhibition of dengue virus growth in OAS3-expressing HepG2 cells. Parental HepG2.Tet-Off cells and HepG2.Tet-Off/OAS3#F8 cell clone in the presence (uninduced) or absence (induced) of 2 μg·mL−1 tetracycline were infected with dengue virus type-1 strain FGA/NA did at different multiplicities of infection. At 40 hours post-infection, cells were fixed with methanol/acetone at −20° C. for 20 min. Immunofluorescence assay was performed using FITC-conjugated anti-dengue E mAb 4E1. Nuclei were stained with DAPI. The percentage of HepG2 cells positive for viral antigens was determined in a triplicate experiment. Virus progeny production was determined at the multiplicity of infection of 25. Virus titration was performed as previously described (Duarte dos Santos et al. Virology, 2000, 274, 292-).

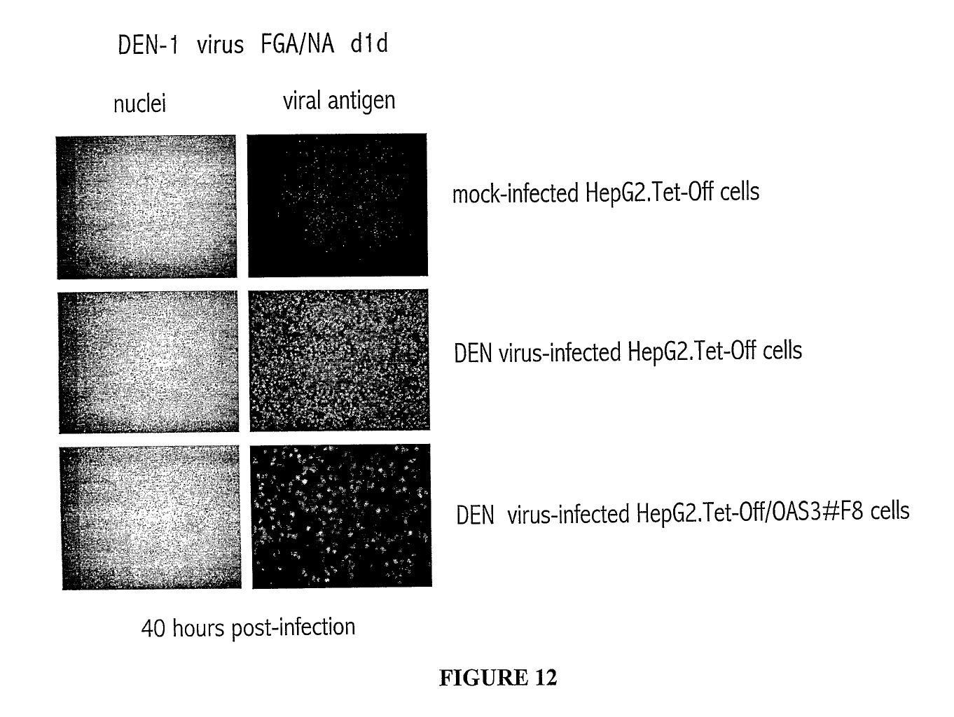

FIG. 12: OAS3-expressing HepG2 cells show resistance to dengue virus infection. Parental HepG2.Tet-Off and induced HepG2.Tet-Off/OAS3#F8 cell clone were infected with dengue virus type-1 strain FGA/NA did (Duarte dos Santos et al., Virology, 2000, 274, 292-) at multiplicity of infection 30 AP61FFU/cell or mock-infected. At 40 hours post-infection, immunofluorescence assay was performed as described in figure legend 11.

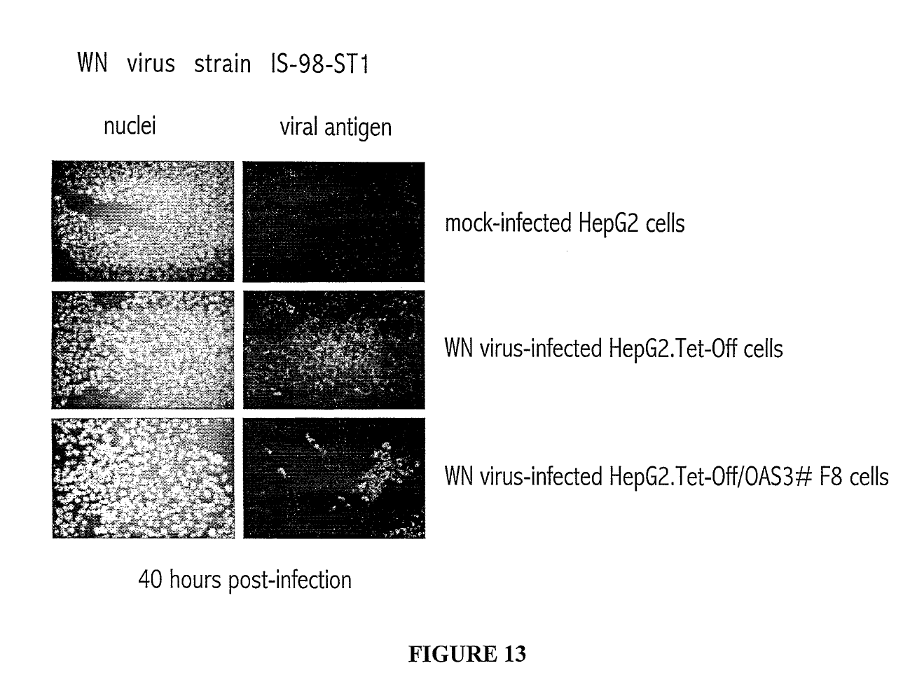

FIG. 13: OAS3-expressing HepG2 cells show resistance to West Nile virus infection. Parental HepG2.Tet-Off and induced HepG2.Tet-Off/OAS3#F8 cell clone were infected with West Nile virus strain IS-98-ST1 (Lucas et al., Virology J., 2004, 1, 9-) at multiplicity of infection 1 AP61FFU/cell or mock-infected. At 40 hours post-infection, cells were fixed with methanol/acetone at −20° C. for 20 min. Immunofluorescence assay was performed using cy3-conjugated anti-West Nile E in Ab E24. Nuclei were stained with DAPI.

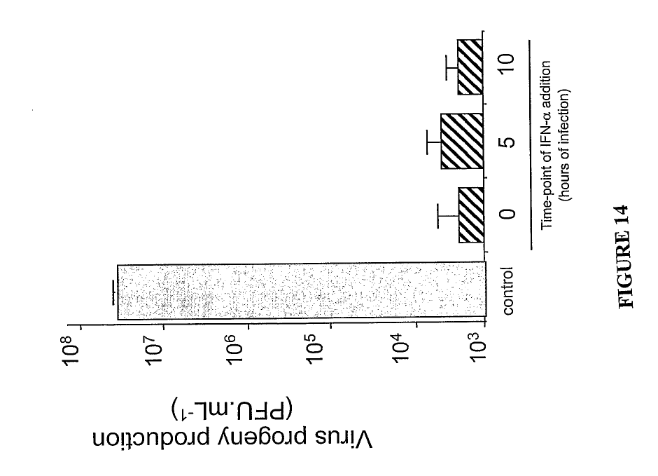

FIG. 14 shows that IFN-α is able to establish an antiviral state against YFV 17D in human epithelial HeLa cells. HeLa cells were infected with YFV 17D at 1 PFU/cell and then treated with 1,000 IU/ml human IFN-α or mock-treated (control) at various time-points post-infection. Virus progeny productions were determined at 72 h post-infection.

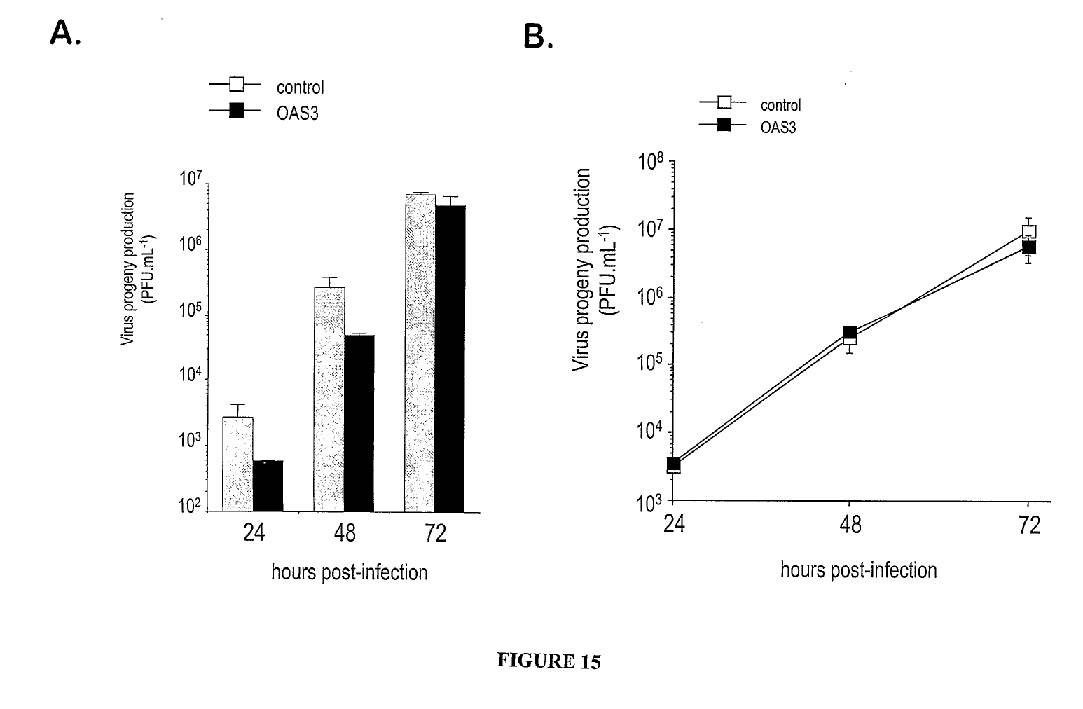

FIG. 15 shows that 17D live-attenuated strain of YFV shows unexpected resistance to OAS3-mediated antiviral effects in human cells regardless either the timepoints of post-infection or the multiplicity of infection. HeLa (control) and induced HeLa.Tet-Off/OAS3 (OAS3) cells were exposed to YFV strain 17D at low (panel A; 1 PFU/cell) or high (panel B; 10 PFU/cell) virus input.

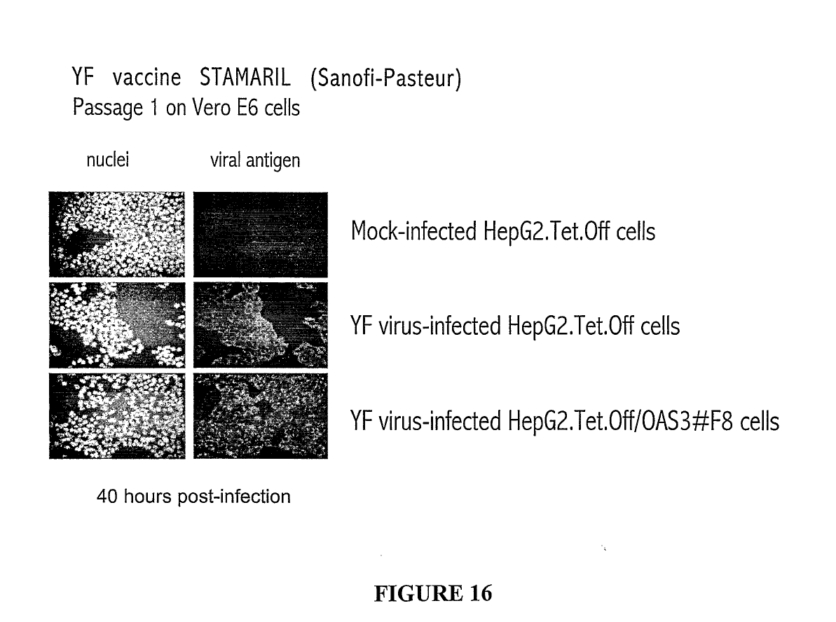

FIG. 16: No inhibition of yellow fever vaccine 17D-204 in OAS3-expressing HepG2 cells. Parental HepG2.Tet-Off and induced HepG2.Tet-Off/OAS3#F8 cell clone were infected with live-attenuated STAMARIL vaccine of yellow fever (Sanofi-Pasteur) grown once on Vero E6 cells or mock-infected. At 40 hours post-infection, cells were fixed with methanol/acetone at −20° C. for 20 min. Immunofluorescence assay was performed using anti-French Neurotropic Virus HMAF and FITC-conjugated goat anti-mouse Ig. Nuclei were stained with DAPI.

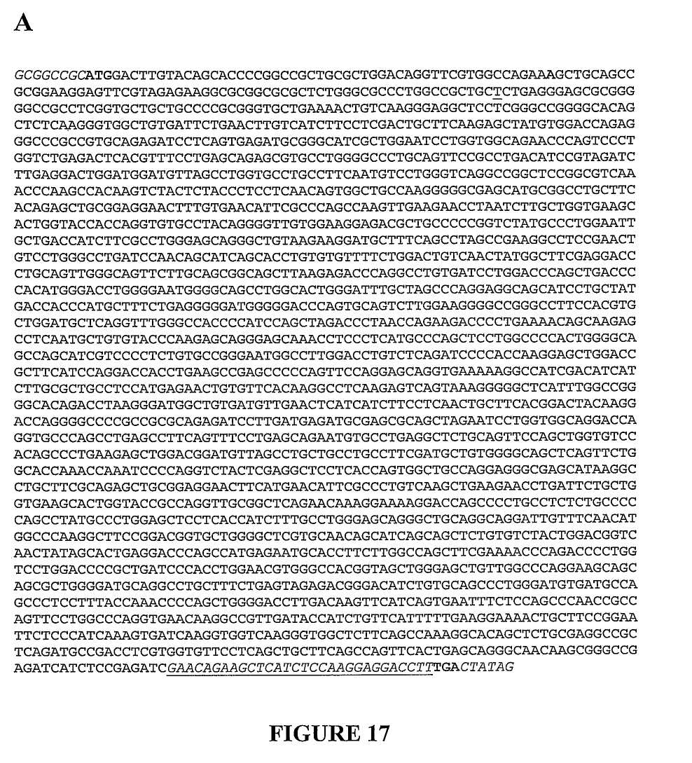

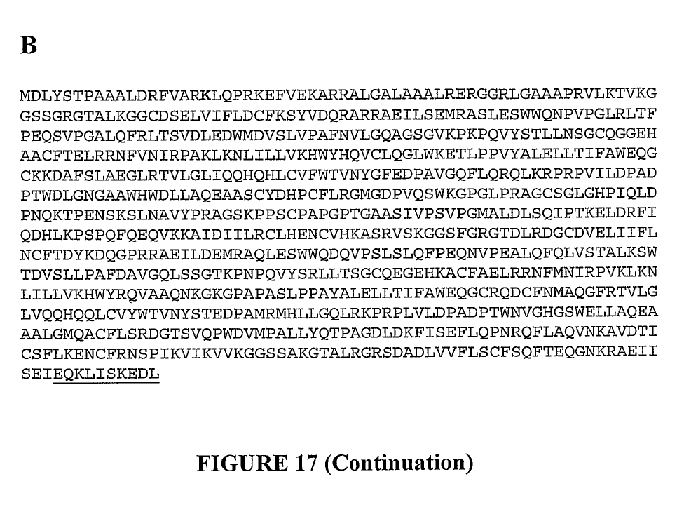

FIG. 17 represents the nucleotide (A) and amino acid (B) sequences of truncated human recombinant OAS3 (OAS31-843). These nucleotide and amino acid sequences correspond to SEQ ID NO: 25 and SEQ ID NO: 26, respectively. Changes between OAS3 mRNA reference sequence and cloned OAS3 cDNA fragment are underlined if synonymous or shown in bold if non synonymous. The translation initiation and stop codons are in bold. The additional nucleotide and amino acid sequences corresponding to the C_terminal c-myc epitope are underlined. The 5′ NotI and 3′ EcoRV restriction sites are shown in italics.

### EXAMPLE 1

### Establishment and Evaluation of HeLaTet-Off/OAS3 Cell Lines

### 1) Material and Methods

a) Cell cultures

The HeLa.Tet-Off cell line was purchased from BD BIOSCIENCES CLONTECH. HeLa.Tet-Off cells are maintained in 5% CO₂ at 37° C., in DMEM (INVITROGEN), supplemented with 10% heat-inactivated fetal calf serum (FCS), 4 mM L-glutamine, 100 UI/ml penicillin, 10 μg/ml streptomycin and 200 μg·mL⁻¹ G418 (INVITROGEN).

The HeLa.Tet-Off/OAS3 cell line (CNCM I-3927) is maintained in DMEM, 10% FCS, 4 mM L-glutamine, 100 UI/ml penicillin, 10 μg/ml streptomycin, 200 μg·mL⁻¹ G418, 100 hygromycin B (BD BIOSCIENCES CLONTECH), and 2 μg·mL⁻¹ tetracycline (SIGMA-ALDRICH). The cells are grown in monolayers; the expected cell density is of 80% to 100%; the population doubling time is of about two days. The cells are harvested by trypsinization; they are sub-cultured 1/10 every week. The cells have a limited lifespan (15 to 20 passages). The cells are frozen in DMEM supplemented with 20% FCS, 10% DMSO, 4 mM glutamine, 100 UI/ml penicillin, 10 μg/ml streptomycin, 200 μg·mL⁻¹ G418, 100 μg·mL⁻¹ hygromycin B, and 2 μg·mL⁻¹ tetracycline.

The HeLa.Tet-Off/OAS3/delta/1C (OAS3^{ΔC-term}) cell line (CNCM I-3968) is maintained in DMEM, 10% FCS, 20 mM L-glutamine, 10,000 UI/ml penicillin, 10 μg/ml streptomycin, 200 μg·mL⁻¹ G418, 100 μg·mL⁻¹ hygromycin B (BD BIOSCIENCES CLONTECH), and 10 μg·mL⁻¹ tetracycline (SIGMA-ALDRICH). The cells are grown in monolayers; the expected cell density is of 90%; the population doubling time is of about two days. The cells are harvested by trypsinization; they are sub-cultured 1/10 every week. The cells have a limited lifespan (25 passages). The cells are frozen in DMEM supplemented with 20% FCS, 10% DMSO, 4 mM glutamine, 10,000 UI/ml penicillin, 10 μg/ml streptomycin, 200 μmL⁻¹ G418, 100 μg·mL⁻¹ hygromycin B, and 10 μg·mL⁻¹ tetracycline.

b) Establishing HeLa.Tet-Off cell lines expressing OAS3 proteins

Two overlapping cDNA fragments of human OAS3 cloned separately in the PCR4-TOPO vector (INVITROGEN), were used as templates for expression of the full-length OAS3 protein (aa 1 to 1,087; SEQ ID NO: 24 and FIG. 1) or the truncated form OAS3^{ΔC-term} or OAS3^{{1-843}} (aa 1 to 843; SEQ ID NO: 26 and FIGS. 3C, 9 and 17). Nucleotide and amino acids differences between reference OAS3 sequence (GenBank accession number NM-006187; SEQ ID NO: 27) and OAS3 cDNA used in this study (FIG. 1) are listed in Table I.

TABLE I
Nucleotide and amino acid changes between OAS3 mRNA reference sequence
and cloned OAS3 cDNA.

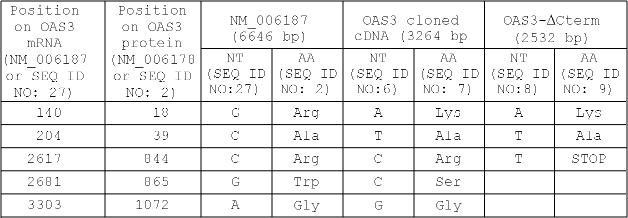

*Differences in translation are highlighted in grey

The OAS3 sequence was modified by PCR to be flanked on the 3′ open reading frame end by the additional 10-residue sequence EQKLISKEDL (SEQ ID NO: 10) followed by a stop-codon with the couple of primers pTet-OAS3-univ1 and pTet-OAS3-rev2 for OAS3 and the couple of primers pTet-OAS3-univ1bis and pTet-OAS3-rev2bis for OAS3^{{1-843}} (Table II).

TABLE II
Primers sequences (SEQ ID NO: 11 to 22)

| primer | Nucleotide sequence (5′ -> 3′) |
|---|---|
| OAS3.R844X- | gtggtgttcctcagctgctt |
| F | |
| OAS3.R844X- | catcgtctgggatgtcagtg |
| R | |
| pTet-OAS3- | attaatgcggccgcaacgaaaccagaaatccgaaggcc |
| univ1 | |
| pTet-OAS3- | aactaactatagtcaaaggtcctccttggagatgagct |
| rev2 | tctgttccacagcagccttcactggccatggctggatg |
| | gg |
| pTet-OAS3- | attaatgcggccgcatggacttgtacagcaccccggcc |
| univ-1bis | gctgcg |
| pTet-OAS3- | aactaactatagtcaaaggtcctccttggagatgagctt |
| rev-3bis | ctgttcgatctcggagatgatctcggcccgccgcttgtt |
| | gcc |
| OAS3-For | cggacggtgctggggctcgtg |
| OAS3-Rev | cttgtcttcgagtagaggcttctc |
| Chik/E2/ | caccgccgcaactaccg |
| 9018/+ | |
| Chik/E2/ | gattggtgaccgcggca |
| 9235/− | |
| GAPDH-For | gggagccaaaaggg |
| GAPDH-Rev | ggggacacggaagg |

Enzyme recognition site are underlined. Sequence complementary to a stop codon are shown in bold. The OAS3 sequences (SEQ ID NO: 23 and 25) are flanked by the NotI and EcoRV restriction enzymatic sites at the downstream and upstream ends, respectively. The PCR products were digested with NotI and EcoRV and then inserted into the unique sites NotI and EcoRV of pTRE2hyg expression vector (BD BIOSCIENCES CLONTECH) to generate pTRE2hyg-OAS3 (FIG. 1) and pTRE2hyg/OAS3CΔ^{C-term} (OAS31-843). In this configuration, the OAS3 insert is under the control of the Tet-Off expression system. The Tet-Off system allows the induction of a foreign gene expression by the withdrawal of repressor tetracycline (Tet). HeLa.Tet-Off cells (BD BIOSCIENCES CLONTECH) were transfected with pTRE2hyg-OAS3 or pTRE2hyg/OAS3CΔ^{C-term}, using transfectant reagent Fugene 6 (ROCHE)) according to the manufacturer's recommended procedure. The Tet-Off expression system was repressed by adding 2 μg/ml of Tet to culture medium. The transfected cells were selected on growth medium containing inhibitors G418 and hygromycin and then cloned from single cells by limiting dilution in presence of 10 μg·mL⁻¹ repressor Tetracycline (Tet). For Tet withdrawal, cell monolayers were trypsined and cells were washed at least five times with non-supplemented DMEM before replacing with DMEM/10% FBS supplemented with genotoxic drugs only. The level of recombinant OAS3 mRNA production in induced cells (−Tet) relative to that in uninduced cells (+Tet) was determined by RT-PCR analysis using the couple of primers OAS3-For and OAS3-Rev (Table II). Based on these experiments, the inducible HeLa.Tet-Off/OAS3#C417-1 and HeLa.Tet-Off/OAS3/delta/1C (OAS3^{ΔC-term}) clones were selected. The HeLa.Tet-Off/OAS3#C417-1 cell line was deposited Feb. 26, 2008, at the Collection Nationale de Cultures de Microorganismes, 25 rue du Docteur Roux, 75724 Paris Cedex 15, under the accession number I-3927. The HeLa-Tet-Off/OAS3/delta/1C cell line was deposited Apr. 17, 2008, at the Collection Nationale de Cultures de Microorganismes, 25 rue du Docteur Roux, 75724 Paris Cedex 15, under the accession number I-3968. Both HeLa.Tet-Off/OAS3 cell lines were maintained under repressing condition in the presence of 2 μg·mL⁻¹ Tet.

### c) Virus

Production of clinical isolate Chikungunya virus strain 06-49 (CHIKV 06-49; Schuffenecker et al., PLoS Medicine 3, 2006, e263) on mosquito Aedes pseudoscutellaris (AP61) cell monolayers and virus titration by focus immunodetection assay (Desprès et al., Virology, 1993, 196, 209-219) were performed as previously described (Bréhin et al., Virology, 2008, 371, 185-195). Infectivity titers were expressed as focus forming units (FFU) on AP61 cells. In order to assay its antiviral effect, human IFN-α (BIOSOURCE) was directly added to culture medium at 1,000 IU·mL⁻¹.

### d) Immunoblot Assays

Cellular proteins were subjected to immunoblot analysis as previously described (Bréhin et al., Virology, 2008, 371, 185-195). Viral protein expression was detected using anti-CHIKV HMAF or anti-CHIK.E2 MAb 3E4 (Bréhin et al., Virology, 2008, 371, 185-195). OAS3 protein expression was detected using anti-OAS3 N-term (Santa-Cruz) or C-term (ABGENT) antibodies.

### e) Flow Cytometry Analysis

Cells were detached and then fixed with 3.2% paraformaldehyde in PBS. Fixed cells were permeabilized, stained with anti-CHIKV HMAF and analyzed by flow cytometry as described previously (Bréhin et al., Virology, 2008, 371, 185-195).

### 2) Results

First, the susceptibility of parental HeLa.Tet-Off cell line to CHIKV infection, was evaluated (FIG. 3A). An analysis of CHIKV replication in these cells infected at 1 multiplicity of infection (MOI) showed that production of progeny virus reached ˜7.0 log FFU·mL⁻¹ at 18 h p.i. By flow cytometry analysis using MAb 3E4 reactive to CHIKV E2 glycoprotein (Bréhin et al., Virology, 2008, 371, 185-195), about 50% of CHIKV-infected HeLa.Tet-Off cells were positive for viral antigens. Then, the ability of IFN-α to establish an antiviral state in HeLa.Tet-Off cells, was investigated. Pretreatment of HeLa.Tet-off cells with 1,000 IU·mL⁻¹ human IFN-α5 hours prior CHIK exposure (1 MOI) resulted in about 1.5 log reduction in virus titer at 18 h p.i. (FIG. 3B). Thus, IFN-dependent antiviral pathways are functional in HeLa.Tet-Off cells and provide protection against CHIKV at cellular level. However, CHIKV-infected cells showed complete resistance to IFN-α at 5 h p.i. once virus replication is established inside infected HeLa.Tet-Off cells.

The stable HeLa.Tet-Off/OAS3#C417-1 cell clone that up-regulates OAS3 protein expression under the control of the Tet-Off expression system was selected to assess the antiviral activity of the large form of OAS. The recombinant OAS3 protein is composed of three adjacent OAS units (domain I, II, and III) including three potential active catalytic sites (FIG. 3C). The expression of recombinant OAS3 protein was analyzed by immunoblotting of cell lysates with a polyclonal immune serum directed against the C-terminal region of human OAS3 protein (FIG. 3D). Cells incubated with 1,000 IU·mL⁻¹ of IFN-α for 5 hours served as a positive control. Upon IFN-α treatment, the production of endogenous OAS3 molecules was clearly detected in HeLa.Tet-Off cells whereas no basal expression of OAS3 was observed in resting cells. Thus, OAS3 gene expression may be actively up-regulated by IFN-α/(3. A basal level of recombinant OAS3 protein was observed in uninduced HeLa.Tet-Off/OAS3 cells (2 μg·mL⁻¹ Tet) indicating that shut-off was incomplete (FIG. 3D). When the repressor Tet was removed 24 h from the culture medium, there was a significant increase in production level of recombinant OAS3 protein compared with that found in uninduced cells.

### EXAMPLE 2

### Antiviral Effects of OAS3 Against CHIKV

### 1) Material and Methods

The experimental procedures are as described in example 1.

### 2) Results

To investigate the effect of OAS3 protein on CHIKV growth in human epithelial cells, HeLa.Tet-Off/OAS3 cells were infected 18 h with CHIKV.06-49 at 1 MOI. As determined by flow cytometry using MAb 3E4, uninduced HeLa.Tet-Off/OAS3 and HeLa.Tet-Off cells displayed similar susceptibility to CHIKV infection (FIG. 4A). Induction of HeLa.Tet-Off/OAS3 cells 24 h prior to virus exposure resulted in reduction of CHIKV-infected cells by at least 20%. As a positive control, IFN-α treatment reduced the percentage of infected HeLa.Tet-Off cells by 75%. Thus, CHIKV is sensitive to ectopic expression of OAS3 in human epithelial cells.

To further assess the efficiency with which HeLa.Tet-Off/OAS3 cells inhibit CHIKV growth, induced cells were exposed to increasing input of CHIKV-06.49 (FIG. 4B). At 18 h p.i., expression of recombinant OAS3 protein reduced the progeny virus production by 1.5 to 2.0 log regardless of the MOI tested. At 24 h p.i., there was about 3.0 log reduction in the viral titer at the lower MOI tested. Thus, expression of OAS3 can suppress the cell-to-cell spread of CHIKV in human fibroblastic cells. At moderate MOI, the virus progeny was still reduced by at least 2.0 log. At 10 MOI, viral growth was reduced only by about 0.5 log suggesting that CHIKV has the ability to avoid the antiviral activities of OAS3 when infection was performed at high virus input.

Kinetic studies showed that OAS3-mediated inhibition of CHIKV was effective during virus life cycle (FIG. 4C). At 12 h p.i., there was a 2.0 log reduction in the viral titer recovered from HeLa.Tet-Off/OAS3 cells as compared with HeLa.Tet-Off cells. Induction of OAS3 protein expression was still able to reduce the progeny virus production by at least 1.5 log at 24 h p.i.

Measurements of Lactate dehydrogenase (LDH), a cytoplasmic enzyme that is released into the culture medium upon cell lysis and therefore is a measure of membrane integrity, showed no significant loss of viability of infected HeLa/Tet-Off/OAS3 cells within the first 24 h of infection as compared to infected parental cells. Thus, the resistance to CHIKV infection was directly related to the antiviral activity of the OAS3 rather than elimination of virus-infected cells through apoptosis induction.

### EXAMPLE 3

### Antiviral Action of OAS3 Against Other Positive-Sense Single-Stranded RNA Viruses in HeLa.Tet-Off Recombinant Cell Line Expressing Human OAS3

### 1) Material and Methods

The experimental procedures are as described in example 1. In addition virus production and titration were performed as described below.

### Viruses

Production of low passaged West Nile Virus (WNV) IS-98-ST1 strain (GenBank accession number AF 481864) on mosquito Aedes pseudoscutellaris (AP61) cell monolayers and virus titration by focus immunodetection assay (Desprès et al., Virology, 1993, 196, 209-219) were performed as previously described (Bréhin et al., Virology, 2008, 371, 185-195; International Application WO 02/081741). Infectivity titers were expressed as focus forming units (FFU) on AP61 cells. Sindbis virus strain AR339 (SINV AR339) and Semliki Forest Virus strain SF 64 (SFV 64) were propagated on African green monkey kidney (VERO) cell line and infectivity titers were expressed as plaque forming units (PFU) on VERO cells. In order to assay its antiviral effect, human IFN-α. (BIOSOURCE) was directly added to culture medium at 1,000 IU·mL⁻¹.

### 2) Results

The ability of the large form of OAS to inhibit growth of SINV strain AR339, SFV strain SF 64 and WNV strain IS-98-ST1 in human epithelial cells was examined (FIGS. 4D, 5 and 6). HeLa.Tet-off and induced HeLa.Tet-Off/OAS3 cells were infected with alphaviruses at 1 MOI. In response to OAS3 protein expression, there was a comparable degree of inhibition (˜2 log) in progeny virus production between SINV and SFV at 18 h p.i (FIG. 4D). HeLa.Tet-off and induced HeLa.Tet-Off/OAS3 cells were infected 48 hours with flavivirus (WNV) at 0.1, 1 or 10 MOI (FIGS. 5 and 6). Based on the measurement of viral titers, it was shown that human epithelial cells respond to OAS3 expression by efficiently inhibiting WN virus replication. At 0.1 MOI, there was a 1.5 log reduction in the viral titer recovered from HeLa.Tet-Off/OAS3 as compared with HeLa.Tet-Off cells (FIG. 6). Induction of OAS3 protein expression was able to reduce the progeny virus production by at least 1.0 log at 72 h post-infection. Thus, OAS3-expressing HeLa cells show lower susceptibility to WN virus infection as compared to parental cells. In conclusion, the data show that OAS3 has antiviral action against RNA viruses such as alphaviruses and flaviviruses inside human cells.

### EXAMPLE 4

### Mechanisms of OAS3-Mediated CHIKV Inhibition

### 1) Material and Methods

The experimental procedures are as described in example 1. In addition quantitative RT-PCR was performed as described below.

### Quantitative RT-PCR

Real-Time RT-PCR analysis of viral RNA accumulation was performed with an ABI Prism 7700 sequence detection using the SYBR® Green PCR essentially as described previously (Kajaste-Rudnistki et al., J. Biol. Chem., 2006, 281, 4624-4637). The primers for CHIKV E2 gene are Chik/E2/9018/+ and Chik/E2/9235/− (Table II). GAPDH mRNA was used as an endogenous sequence control for the normalization of each sample. The primers GAPDH-For and GAPDH-Rev are listed in Table II. For the quantification of viral RNA copies, an in vitro CHIK RNA transcript encoding the N-terminal region of E2 was performed to build the standard curve as previously described (Vazeille et al., PLoS One, 2007, 2(11): e1168).

### 2) Results

In an effort to resolve the molecular basis of the antiviral action of OAS3, it was determined whether inhibition of CHIKV growth was due to a lack of accumulation of structural virus proteins. Total proteins were extracted from Hela.Tet-off and induced HeLa.Tet-Off/OAS3 cells at the 18-h timepoint post-infection and capsid protein (C), envelope glycoproteins pE2 (precursor of E2), E2, and E1 were detected by immunoblot using anti-CHIKV HMAF, whereas envelope glycoproteins pE2 (precursor of E2) and E2 were detected specifically by immunoblot using anti-CHIKV E2 MAb 3E4. As shown in FIG. 7A, there was a background level of structural virus proteins in response to OAS3 protein expression. Thus, the inability of CHIKV to productively infect OAS3-expressing HeLa cells is associated with a severe reduction in viral protein synthesis.

It was determined whether the inefficiency of viral protein synthesis was due to a lack of accumulation of viral RNA. Total RNA was extracted from Hela.Tet-off and induced HeLa.Tet-Off/OAS3 cells at the 8 h time-point post-infection and production of genomic and subgenomic viral RNAs was analyzed using RT-PCR assay with primers designed on the basis of CHIKV E2 gene. As determined by real-time RT-PCR analysis, there was a 2 log reduction in viral RNA level recovered from OAS3-expressing cells when compared to that found in parental cells (FIG. 7B). Thus, OAS3 expression prevents viral RNA accumulation inside infected cells. On the basis of these results, it can be concluded that OAS3 expression affects alphaviral replication inside infected human cells.

### EXAMPLE 5

### Impact of OAS3 Genetic Polymorphism in Healthy Individuals on OAS3 Antiviral Effect

### 1) Material and Methods

The experimental procedures are as described in example 1. In addition, genotyping of OAS3 polymorphism was assayed as described below.

### Genotyping

The SNP OAS3.R844X was genotyped by PCR-RFLP assay using the couple of primers OAS3.R844X-F(SEQ ID NO: 11) and OAS3.R844X-R (SEQ ID NO: 12) which are presented in Table II. The 183 bp-long PCR product was subjected to digestion with Bgl II and the detection of two fragments of 115 and 68 bp indicated the presence of OAS3.R844X. 2) Results

The OAS3 gene was screened for polymorphisms. Re-sequencing total 16 exons, flanking introns and 2 kb 5′ to the OAS3 gene in fourty-eight healthy Caucasians individuals identified a single-nucleotide polymorphism (SNP) at the first position of codon CGA-844 where the substitution T for C resulted in non-sense mutation (OAS3.R844X) (Table I). This SNP was further screened in 180 healthy Caucasian individuals and identified two heterozygotes which gave allele frequency of 0.5%. Because OAS3.R844X possibly truncates about 20% of the OAS3 protein from the carboxy terminus (FIG. 3C), the stable HeLa.Tet-off/OAS3^{ΔC-term} cell clone which contains the OAS3-1 to OAS3-843 sequence, was generated. It is of interest to note that OAS3^{ΔC-term} lacks the D⁸⁸⁹ residue which composes the triad defining the active catalytic site of the third domain of OAS3 (Sarkar et al., 1999; Rambouillat et al., 2000). To assess the ability of mutant OAS3 to inhibit CHIKV and WNV growth, induced HeLa.Tet-Off/OAS3 and HeLa.Tet-off/OAS3^{ΔC-term} cells were infected at 1 MOI with either CHIKV-06-49 or West Nile Virus (WNV) IS-98-ST1 strain. As shown in FIGS. 8 and 9, OAS3^{ΔC-term}-expressing cells displayed resistance to CHIKV and WNV infection indicating that OAS-1 to OAS3-843 sequence is still able to provide protection. Analysis of viral growth showed that ectopic expression of OAS3^{ΔC-term} resulted in a lower efficiency of CHIKV and WNV inhibition as compared to full-length OAS3. Such result suggests that SNP at codon CGA-844 may have an impact on the antiviral activity.

### EXAMPLE 6

### Antiviral Action of OAS3 Against DV and WNV in Hep.G2.Tet-Off Cell Line Expressing Human OAS3

### 1) Material and Methods

### a) Cell Culture

The HepG2.Tet-Off cell line was purchased from CLONTECH (# 632106). HepG2.Tet-Off cells are maintained in 5% CO₂ at 37° C., in DMEM (GIBCO, # 41965), supplemented with 10% heat-inactivated fetal calf serum (FCS), 4 mM L-glutamine, 100 UI/ml penicillin G sodium, 100 μg/ml streptomycin sulfate (GIBCO, # 15140-122), 0.1 mM non-essential amino acids, and 100 μg·mL⁻¹ G418 (GIBCO, # 10131-019).

The HepG2.Tet-Off/OAS3#F8 cell line (CNCM I-4158) is maintained in DMEM, 10% FCS, 4 mM L-glutamine, 100 UI/ml penicillin, 100 μg/ml streptomycin, 100 μg·mL⁻¹ G418, 100 μg·mL⁻¹ hygromycin B (CLONTECH, # 631309), and 2 μg·mL⁻¹ tetracycline (SIGMA-ALDRICH, # T-7660).

### b) Establishing HepG2.Tet-Off Cell Lines Expressing OAS3 Protein

An inducible HepG2.Tet-Off/OAS3#F8 clone expressing the full-length human OAS3 protein (SEQ ID NO: 24) was selected as described in example 1 for the inducible HeLa.Tet-Off/OAS3#C417-1. The HepG2.Tet-Off/OAS3#F8 cell line was deposited May 15, 2009, at the Collection Nationale de Cultures de Microorganismes, 25 rue du Docteur Roux, 75724 Paris Cedex 15, under the accession number I-4158

This cell line is maintained under repressing condition in the presence of 2 μg·mL⁻¹ Tet.

### c) Virus

Production of low passaged West Nile Virus (WNV) IS-98-ST1 strain (GenBank accession number AF 481864; Lucas et al., Virology J., 2004, 1, 9-) on mosquito Aedes pseudoscutellaris (AP61) cell monolayers and virus titration by focus immunodetection assay (Desprès et al., Virology, 1993, 196, 209-219) were performed as previously described (Bréhin et al., Virology, 2008, 371, 185-195). Production of DV1 strain FGA/NA d1d and virus titration were performed as previously described (Duarte dos Santos et al. Virology, 2000, 274, 292-). Infectivity titers were expressed as focus forming units (FFU) on AP61 cells. In order to assay its antiviral effect, human IFN-α (BIOSOURCE) was directly added to culture medium at 1,000 IU·mL⁻¹.

### d) Immunofluorescence Assay

Virus-infected cells were fixed with methanol/acetone at −20° C. for 20 min. Immunofluorescence assay was performed using FITC-conjugated anti-dengue E mAb 4E1 or cy3-conjugated anti-WNV.E MAb E24. Nuclei were stained with DAPI.

### 2) Results

First, the susceptibility of parental HepG2.Tet-Off cell line to DV infection was evaluated (FIG. 10A). Cells were infected with different Focus Forming Units (AP61FFU) per cell of DV-1 virus strain FGA/NA d1d. At 40 h post-infection, the cells were analyzed by flow cytometry using MAb 4E11 reactive to DV E glycoprotein. With a DV-1 virus input of 30 AP61FFU per cell, about 50% of HepG2.Tet-Off cells were infected with DV-1 virus as evidenced by a positive signal for the dengue E glycoprotein.

Then, the ability of IFN-α to establish an antiviral state in HepG2.Tet-Off cells was investigated. Pretreatment of HepG2.Tet-off cells with 1,000 IU·mL⁻¹ human IFN-α5 hours prior DV infection (10 MOI) resulted in about 0.7 log reduction in virus titer at 40 h p.i. (FIG. 10B). Thus, IFN-dependent antiviral pathways are functional in HepG2.Tet-Off cells and provide protection against DV at cellular level.

To investigate the effect of OAS3 protein on DV growth in Hep.G2 cells, induced (−Tet) and uninduced (+Tet) HepG2.Tet-Off/OAS3 cells and parental HepG2.Tet-Off cells were infected with increasing input of DV-1 virus strain FGA/NA d1d. At 40 h p.i., immunofluorescence assay using anti-dengue E mAb was performed and the percentage of HepG2 cells positive for viral antigen was determined (FIGS. 11 and 12). The progeny virus production was determined at the multiplicity of infection of 25 (FIG. 11). Expression of recombinant OAS3 protein reduced the percentage of DV-infected cells by at least 75% (FIGS. 11 and 12) and progeny virus production by 2 log(FIG. 11).

Thus, OAS3-expressing HepG2 cells show resistance to DV infection.

The effect of OAS3 protein on WNV growth in Hep.G2 cells was also analyzed by immunofluorescence assay. Parental HepG2.Tet-Off and induced HepG2.Tet-Off/OAS3#F8 cell clone were infected with West Nile virus strain IS-98-ST1 at multiplicity of infection 1 AP61FFU/cell or mock-infected. At 40 h post-infection viral antigen (WNV E glycoprotein) was detected by immunofluorescence using cy3-conjugated mAb E24. The results (FIG. 13) confirmed those obtained in HeLa cells (example 3), showing that OAS3-expressing human cells show resistance to WNV infection.

### EXAMPLE 7

### Live-Attenuated 17D-204 Vaccine Strain of Yellow Fever Virus Shows Surprising Resistance to the Antiviral Effects Mediated by the Large Form of Human 2′,5′-Oligoadenylate Synthetase (OAS3) in Human Cells

### 1) Material and Methods

Attenuated Yellow Fever Virus (YFV) vaccine strain (STAMARIL®, AVENTIS-PASTEUR) was propagated on African green monkey kidney (VERO) cell line and infectivity titers were expressed as plaque forming units (PFU) on VERO cells. In order to assay its antiviral effect, human IFN-α(BIOSOURCE) was directly added to culture medium at 1,000 IU·mL⁻¹. Immunofluorescence assay was performed as described in example 6, using anti-French Neurotropic Virus HMAF and FITC-conjugated goat anti-mouse Ig.

### 2) Results

Examples 2, 3, 4 and 6 provided the first evidence that OAS3-dependent antiviral activity mediated by IFN-α/β represents a major human cell defence strategy against alphaviruses such as Chikungunya virus and flaviviruses such as West Nile and dengue viruses in human cells.

It was investigated whether OAS3 displays antiviral activity against yellow fever virus (YFV). For this, live-attenuated 17D-204 vaccine strain of YFV (STAMARIL, Aventis-Pasteur) were twice propagated on African green monkey kidney (VERO) cell line and infectivity titers were expressed as Plaque Forming Units (PFU) on VERO cells. HeLa cells were infected with YFV 17D at 1 PFU/cell and then treated with 1,000 IU/ml human IFN-α or mock-treated (control) at various time-points post-infection. Virus progeny productions were determined at 72 h post-infection. As shown in FIG. 14, IFN-α is able to establish an antiviral state against YFV 17D in human epithelial HeLa cells. To assess the ability of OAS3 to inhibit viral growth, HeLa (control) and induced HeLa.Tet-Off/OAS3 (OAS3) cells were exposed to YFV strain 17D at low (1 PFU/cell; FIG. 15A) or high (10 PFU/cell; FIG. 15B) virus input. There was no obvious differences in YFV replication between both cell populations (FIG. 15). Kinetic studies showed that 17D is resistant to OAS3-mediated antiviral effects in human cells regardless either the time points of post-infection or the multiplicity of infection.

The resistance of YFV 17D to OAS-3 mediated antiviral effect was confirmed by immunofluorescence assay in parental HepG2.Tet-Off and HepG2.Tet-Off/OAS3#F8 cell clone infected with YFV 17D (FIG. 16).

The observation that live-attenuated strains of YFV show unexpected resistance to antiflaviviral action of OAS3 opens a new avenue for elucidating the mechanism of flavivirus attenuation.

### EXAMPLE 8

### A Non-Synonymous Variant of OAS3 is Associated with the Pathogenesis of Dengue Shock Syndrome

### 1) Material and Methods

a) Patients and controls

750 patients (male:female ratio=0.99) with symptomatic dengue viral (DV) infection during the 3 year period (June 2000-2003) were enrolled in the study from two medical centers in Bangkok, Ramathibodi Hospital and Siriraj Hospital, Mahidol University and 1 hospital in Khon-Kaen province, Thailand. Their ages ranged from 1 to 25 years with a mean of 9.6 years. Patients with suspicion of dengue viral infection based on clinical features including high fever, severe headache, retro-orbital pain, myalgia, arthalgia, nausea and vomiting, and rash were admitted to the hospitals for clinical observation and treatment. The diagnosis of dengue virus infection was later confirmed by a comparable IgG and IgM enzyme-linked immunosorbent assay titer on a late acute and/or convalescent sera. Differential diagnosis of DF (Dengue fever) and DHF (Dengue Hemorrhagic Fever) was established based on the absence (DF) or presence (DHF) of evidence for increased vascular permeability manifested by hemoconcentration or pleural effusion. Specifically, the diagnosis of DHF was made based on all of the four following characteristics: 1) high continuous fever lasting 2-7 days, 2) hemorrhagic tendency such as a positive tourniquet test, petechii, purpura or hematemesis, 3) thrombocytopenia (platelet count≦100,000/μl and 4) evidence of plasma leakage due to increased vascular permeability manifested by hemoconcentration (an increased in hematocrit of 20% or more) or pleural effusion. Some dengue patients who could not be classified as DF or DHF because of unclear clinical symptoms were assigned an unknown DF/DHF status. The severity of DHF was categorized by four grades according to WHO criteria. Grades III and IV were DHF with narrowing pulse pressure with a characteristically elevated diastolic pressure to profound shock. Secondary infection was defined as a dengue-specific IgM/IgG ratio<1.8. The project protocol and objectives of the study were carefully explained to the patients and their parents or relatives. Informed consent was individually obtained from all subjects. The protocol has been approved by the ethical committee of each hospital.

The controls consisted of 296 blood donors from Ramathibodi hospital and 216 blood donors from Siriraj Hospital and 184 ethnic matched healthy controls from Khon-Kaen region (male:female ratio=1). Both cases and control groups came from Bangkok and Central part of Thailand. Whole blood samples were collected from patients and controls on EDTA, and DNA was extracted using a standard phenol/chloroform extraction method.

During 2004-2006, we enrolled additional 254 patients (male:female ratio=1.07) from Ramathibodi and Khon-Kaen hospital using the same clinical and viral diagnosis criteria.

### b) Polymorphisms Identification and Genotyping

Polymorphisms were identified by direct sequencing of PCR amplified genomic DNA, on individual DNA samples (24 dengue patients and 32 Thai controls). Primers used for sequencing OASs exons, part of introns and 5′ and 3′ regions are shown in Table III.

TABLE III
OAS3 sequencing primers (SEQ ID NO: 31 to 142)

| | | | PCR |
|---|---|---|---|
| | Forward primer | Reverse primer | product |
| gene/exon | sequence | sequence | size |
| OAS1/pro1 | CCAGGAGTTCGAGACCAGAC | TGCAGGGACAGTCACAGAAC | 546 |
| OAS1/pro1 | GGTGAGGCATAGGGGATTTT | ATAGGAGGTGGGGCTTGACt | 409 |
| OAS1/pro3 | CCTTGAACCCAGGAAGTTGA | CCTTTGTCCTTTAGCCAGCA | 498 |
| OAS1/pro4 | CTTTTTGCAGTGGGCATGTA | TGTCAATGGCATGGTTGATT | 503 |
| OAS1/exon1 | TTTGCAAAAGGAAAGTGCAA | GAAACCACCATCCCAGAAGA | 503 |
| OAS1/exon2 | AGCATCCATTTTCCCATCTG | CCCACCCTGCTTTAGAGAGA | 604 |
| OAS1/exon3 | GGATCAGGAATGGACCTCAA | GGCTCCTCTCTCCACCTCTT | 499 |
| OAS1/exon4 | AATGAATGAGCCTGGATTCG | TACAATGATGGGCAACAGGA | 493 |
| OAS1/exon5 | GAGCCCTTCCTCATGTTCTG | ATAAATGGCCTTTGGCAAGA | 396 |
| OAS1/exon6-1 | AGCTGGGGCTTGTTAGTCCT | TGGAATGAAGTGAGGCTGTG | 502 |
| OAS1/exon6-2 | GCTCCTCAGTGAGCTGGTGT | TCCATGAGGACAGGGATTTT | 508 |
| OAS1/exon6-3 | CGGCCATGTCATTGTTTTTAT | ACTGCCAGAGTGGAGATGCT | 560 |
| OAS2/pro1 | AAAGCACAGGCTTTGGAGTC | TGAGCCCAGGTACCCAGATA | 600 |
| OAS2/pro2 | TTGGGGGAGCTCAGTCTTTA | CTGACTTGCTCAGGGACACA | 583 |
| OAS2/pro3 | TTTCTGGCTCACACACTTGG | AGCAGGGAAACCAAAACTGA | 602 |
| OAS2/pro4 | CGATGTGCTCAAGGACAGAC | CCATTTCCCATTGCTCTCAG | 480 |
| OAS2/exon1 | CCTCCCATCCTACCATTCAC | GTCACACAGCTCTGGAGCAA | 394 |
| OAS2/exon2 | ATGAGCATCCCAATCAGGTC | GAGAGCGAGTCCAGGGTAGA | 597 |
| OAS2/exon3 | TGACCGATTAAGCAAGAGTCG | TGTTGCACTTTACTGGATGTCA | 379 |
| OAS2/exon4 | TAAGAGAGGTCCCTGCTCCA | GGACCTCCCCTCTTTCAGAC | 595 |
| OAS2/exon5 | GAGGGGCTTGATGTCGTAGA | CTCTGCCAACACCCTGATTT | 511 |
| OAS2/exon6 | TTGTGTGCCACTCCAAACTC | TAACTCCTGGCTTCCCTTCC | 500 |
| OAS2/exon7 | CAGAACTTGGCCTTGGAAAC | CCCATGCTTGGAAAAGAGAA | 594 |
| OAS2/exon8 | GGAGATGCTCCCTGTGTCTT | GGGTTAGTGATGGGAACGTC | 499 |
| OAS2/exon9 | TCCAAGCTGCAGAGTGTGAG | GCCCCTGCTAGGTTTTATCC | 532 |
| OAS2/exon10-1 | CGCGGCCTCTAGAAAGTTAC | GGTTGTCACTGGGAGAGGAG | 593 |
| OAS2/exon10-2 | AGGCAGGTAACCCCAGATTC | GCACAAGAGAGCCTCCAAAC | 606 |
| OAS2/exon10-3 | TGGTTCTCTCCACCAGGTTC | GGATGGATCCTAGCTCCACA | 496 |
| OAS2/exon11-1 | GTAGGGAAGAGGTGGCCAAG | GACTGGAAGGAAAAAGGAGGA | 600 |
| OAS2/exon11-2 | GTCTTGCCTTGCTGAACTCC | AAGGCTAAAGCCCAAGGAAA | 484 |
| OAS2/exon11-3 | CCAGCCAACCCTTTCATTAG | CACATGCTTGGTCTTTCTGG | 450 |
| OAS3/pro1 | TATTGACCCTTCTGCGTTCC | GCACACCTGAGCTCTCTTCC | 607 |
| OAS3/pro2 | GGAGAGAGGACCTGGGAGTT | CGACCATGGTGTAGGTGCTA | 602 |
| OAS3/pro3 | CCAGCCAGACAACTTTCCAG | TCGGATTTCTGGTTTCGTTT | 609 |
| OAS3/exon1 | AAGTGGGTGTCAGGTCCAAG | TGTCAAGCGGGCTTAAGAGT | 615 |
| OAS3/exon2 | ACAGTCTCTCCCCAGCACAC | CCCAGGTCAAACAGCAAGTT | 490 |
| OAS3/exon3 | AAAGCTCCAGGCTCTCTTCC | TGCACAAGGGACATAAACAGA | 481 |
| OAS3/exon4 | GGCTGAAGCTACCAGTGAGG | CTAATGTGCATCTGGGCTGA | 580 |
| OAS3/exon5 | TCGAGGCTCAGAGAGGGTAA | GCCTCCCAATGTGTTGAGAT | 451 |
| OAS3/exon6 | GGCACCTTTTTCAACTCTGC | CCAGCAAATCATCCTTCCTG | 654 |
| OAS3/exon7 | CATGCAGTCGACCTTTGTCA | TCAATGCTTGGTCAAGTAGGAG | 545 |
| OAS3/exon8 | CCACTGTGCAGTTACTGGATG | AAGCAAAGGAGGGGAGATGT | 508 |
| OAS3/exon9 | CTAGGCAAAAGCCACGTCTG | GGAAGCAGAGTGAGGAGTGG | 606 |
| OAS3/exon10 | CCCAATAGCTTTCCAACCAA | GCTGGGACGTAGCAATCTGT | 500 |
| OAS3/exon11 | CCTCAGAACCTACCCACCAG | GGACATATCTGGGCCCTCTC | 494 |
| OAS3/exon12 | TGACTTGTCCAAGGTCACACA | AAAGGGGCCCTCTTCTTTAG | 566 |
| OAS3/exon13 | ATCCCAGACCACCCTTAGGA | CTCCACCAACCCAGGATCT | 407 |
| OAS3/exon14 | AAAGTTTTGGGGTTGCTTTG | ATTGACCGTTTGTCCCTCAG | 490 |
| OAS3/exon15 | TCTAGCCCCTGCAAAGTGTT | ATGGGAAAATGGAAGCACAG | 395 |
| OAS3/exon16-1 | ATCTGTGGTGCCAAAGGAAG | GCAGGAGGAAAGATGTGAGC | 591 |
| OAS3/exon16-2 | CCTCCCATGGCTTACACACT | CATAGCATGCATGTCCCAAC | 599 |
| OAS3/exon16-3 | CAGGAGAGCATGCCCATATT | GAGAGCCTCCTGGATGGAC | 259 |
| OAS3/exon16-4 | GAGATTCTGCATCCCCACAG | TAGACCCACTCCCTCATCCA | 609 |
| OAS3/exon16-5 | CTCTGGCTGCAGAGGAGTCT | GCCCTGAGCTCTTGGATATG | 597 |
| OAS3/exon16-6 | AACGCTAAGGGCACCTTCTT | GGGTGTTTTTAGGGTGACCA | 603 |
| OAS3/exon16-7 | CACCCTGGTGTTGGCATATT | AGAAGGGTGAGAGCTTTATGG | 634 |

Sequencing results were analyzed by Genalys Software (Takahashi et al., J. Bioinform. Comput Biol., 2003, 1, 253-265). OAS3-R381S was genotyped by TaqMan assay using ABI Prism 7000 Sequence Detection System, with recommended protocols. The codon 381 of the more common variant of OAS3 sequence is AGG which corresponds to a arginine. The polymorphism OAS3-R381S, named rs2285933, has a G to C substitution at the third nucleotide of codon 381 of OAS3 (AGG to AGC) that changes amino acid from arginine to serine. The 2 variants of OAS3-(R381 and S381) mRNA, ORF and amino acid sequences correspond to SEQ ID NO: 28, 29, 30 (R381) and SEQ ID NO: 27, 1 and 2 (S381), respectively.

### c) Statistical Analyses

We performed association study using allele count by Pearson chi-square test implemented in the Haploview program (Barrett et al., Bioinformatics, 2005, 21, 263-265). This program allows us to perform permutation test to obtain an empirical P value. We performed logistic regression analysis coding the genotype results according to the genetic model tested using STATA version 9.

### 2) Results

During 2000-2003, we have recruited 580 dengue patients from two hospitals (Siriraj, S I and Ramathibodi, R A) in Bangkok, and 170 from another hospital in Khonkaen province (KK), north-east of Thailand. We collected DNA samples from 512 blood donors from the same hospitals in Bangkok and 184 healthy volunteer from the north-east region of Thailand to represent gene frequency in general populations. Genetic study of part of these patients and controls has been reported previously (Sakuntabhai et al., Nature Genetics, 2005, 37, 507-513). Viral diagnosis was confirmed by serologic tests, and patients were classified into 4 groups according to WHO criteria: DF (no evidence of plasma leakage), and DHF1 (evidence of plasma leakage), DHF2 (with spontaneous bleeding) and DSS (with shock) (Table IV).

TABLE IV
Disease severity in the studied population

| | 2001-2003 | | | | 2004-2006 | | |
|---|---|---|---|---|---|---|---|
| Clinical | Hospital | | | | Hospital | | |
| Diagnosis | Siriraj | Ramathibodi | Khonkaen | Total | Ramathibodi | Khonkaen | Total |
| DF | 115 | 62 | 49 | 226 | 29 | 70 | 99 |
| DHF1 | 36 | 60 | 41 | 137 | 23 | 21 | 44 |
| DHF2 | 78 | 97 | 48 | 223 | 33 | 29 | 62 |
| DSS | 43 | 46 | 24 | 113 | 15 | 20 | 35 |
| unclassified | 7 | 36 | 8 | 51 | 1 | 13 | 14 |
| Total | 279 | 301 | 170 | 750 | 101 | 153 | 254 |

There were 51 patients who could not be classified as having DF or DHF according to WHO criteria (unclassified; UC). Proportion of patients with each severity category was significantly different among hospitals (P=1.5×10⁻⁸). This reflects difference in type of hospital (secondary care for KK or medical school for SI and RA) and inclusion criteria (fever less than 3 days in KK and suspected dengue cases in SI and RA).

There was an highly significant increased risk of plasma leakage (DHF1/DHF2/DSS versus DF) and spontaneous bleeding (DHF2/DSS versus DF/UC/DHF1) in patients who had a predominant IgG response compared to patients with predominant IgM response to dengue infection (P=1.1×10⁻⁷ and P=7.6×10⁻⁵, respectively) but only marginal significance for shock (DSS versus the rest, P=0.038) (Table V).

TABLE V
Disease severity and immune response

Immune Response

| Clinical Diagnosis | IgM predominant | IgG predominant |
|---|---|---|
| DF | 51 | 162 |
| % | 53.7 | 27.1 |
| DHF1 | 15 | 121 |
| % | 15.8 | 20.2 |
| DHF2 | 19 | 199 |
| % | 20 | 33.3 |
| DSS | 8 | 99 |
| % | 8.4 | 16.6 |
| unclassified | 2 | 17 |
| % | 2.1 | 2.8 |
| Total | 95 | 598 |

These findings are consistent with the notion that individuals with IgG response to DV are predisposed to the more severe forms of the disease.

For screening of tagged polymorphisms, tests for association were performed using allele count on the risk of plasma leakage (DHF1/DHF2/DSS versus DF), risk of spontaneous hemorrhage (DHF2/DSS versus DF/UC/DHF1), risk of shock (DSS versus non-shock DV patients) in patients from the 2 hospitals in Bangkok (Table VI).

TABLE VI
Association studies of OAS3-R381S in dengue patients

| | | | | | | | | Case | Case, | |
|---|---|---|---|---|---|---|---|---|---|---|
| OAS3- | Genotype | | | Freq | Allele | | | versus (vs) | Control | |
| R381S | GG | CG | CC | CG + CC | G | C | MAF* | Control | Ratios | p |
| Siriraj | | | | | | | | | | |
| control | 162 | 49 | 3 | 0.24 | 373 | 55 | 0.13 | | | |
| DF | 57 | 29 | 3 | 0.36 | 143 | 35 | 0.20 | Leak vs NoLeak | 38:276, 46:190 | 0.02 |
| DHF1 | 25 | 11 | 0 | 0.31 | 61 | 11 | 0.15 | Bleed vs NoBleed | 27:215, 59:263 | 0.02 |
| DHF2 | 56 | 21 | 1 | 0.28 | 133 | 23 | 0.15 | Shock vs NoShock | 4:82, 82:390 | 0.0027 |
| DSS | 40 | 2 | 1 | 0.07 | 82 | 4 | 0.05 | | | |
| Rama | | | | | | | | | | |
| control | 210 | 82 | 4 | 0.29 | 502 | 90 | 0.15 | | | |
| DF | 24 | 11 | 2 | 0.35 | 59 | 15 | 0.20 | Leak vs NoLeak | 59:349, 21:103 | 0.50 |
| DHF1 | 42 | 18 | 0 | 0.30 | 102 | 18 | 0.15 | Bleed vs NoBleed | 41:247, 54:260 | 0.32 |
| DHF2 | 66 | 28 | 2 | 0.31 | 160 | 32 | 0.17 | Shock vs NoShock | 8:84, 86:420 | 0.04 |
| DSS | 39 | 6 | 1 | 0.15 | 84 | 8 | 0.09 | | | |
| SirirajRama | | | | | | | | | | |
| control | 372 | 131 | 7 | 0.27 | 875 | 145 | 0.14 | | | |
| DF | 81 | 40 | 5 | 0.36 | 202 | 50 | 0.20 | Leak vs NoLeak | 97:625, 67:293 | 0.0253 |
| DHF1 | 67 | 29 | 0 | 0.30 | 163 | 29 | 0.15 | Bleed vs NoBleed | 68:462, 113:523 | 0.0204 |
| DHF2 | 122 | 49 | 3 | 0.30 | 293 | 55 | 0.16 | Shock vs NoShock | 13:169, 168:816 | 7 · 10⁻⁴ |
| DSS | 79 | 8 | 2 | 0.11 | 166 | 12 | 0.07 | | | |
| Khonkaen | | | | | | | | | | |
| Control | 134 | 45 | 5 | 0.27 | 313 | 55 | 0.15 | | | |
| DF | 23 | 7 | 0 | 0.23 | 53 | 7 | 0.12 | Leak vs NoLeak | 33:181, 12:74 | 0.75 |
| DHF1 | 30 | 8 | 1 | 0.23 | 68 | 10 | 0.13 | Bleed vs NoBleed | 23:113, 27:153 | 0.64 |
| DHF2 | 26 | 17 | 1 | 0.41 | 69 | 19 | 0.22 | Shock vs NoShock | 4:44, 46:222 | 0.12 |
| DSS | 20 | 4 | 0 | 0.17 | 44 | 4 | 0.08 | | | |
| Total | | | | | | | | | | |
| control | 506 | 176 | 12 | 0.27 | 1188 | 200 | 0.14 | | | |
| DF | 104 | 47 | 5 | 0.33 | 255 | 57 | 0.18 | Leak vs NoLeak | 130:806, 79:367 | 0.06 |
| DHF1 | 97 | 37 | 1 | 0.28 | 231 | 39 | 0.14 | Bleed vs NoBleed | 91:575, 140:676 | 0.07 |
| DHF2 | 148 | 66 | 4 | 0.32 | 362 | 74 | 0.17 | Shock vs NoShock | 16:210, 214:1032 | 0.0001 |
| DSS | 99 | 12 | 2 | 0.12 | 210 | 16 | 0.07 | | | |

*MAF: Mean Allele Frequency

As shown in Table VI, none of the polymorphisms showed evidence of association with the risk of disease. However, one non-synonymous polymorphism of OAS3 had highly significantly different allele count distributions depending on the severity of the disease. This variant (OAS3-R381S: rs2285933) is a G to C substitution at the 3^{rd} nucleotide of codon 381 of OAS3, that changes amino acid from arginine to serine. In both hospitals, the C allele of this polymorphism was more frequent in DF, DHF1 and DHF2, than in DSS. There was no significance difference in allele frequency between each group of patients and controls between the 2 hospitals. In combined analyses, the C allele had a frequency of 14% in general population, 20% in DF, and 15-16% in DHF1 and DHF2 and 7% in DSS. The difference between DSS and non-shock DV cases was highly significant (P=7.10⁻⁴). We performed 10,000 permutations and obtained an empirical P value of 0.0032.

### Mode of Inheritance

In order to find out mode of inheritance of the OAS3-R381S, we compared the likelihood of the models based on 3 different mode of inheritance: additive, dominant and recessive of the C allele using logistic regression on the risk of DSS. While recessive model of the C allele did not show significant association, highly significant level was obtained with dominant model of the C allele (<10⁻⁵ P<10⁻⁵, OR=0.30 95CI=0.17-0.53) and additive (17.93, P<10⁻⁵). By log likelihood ratio test, the dominant model is significantly fitter than the additive model (P=0.04). We therefore used dominant model for the rest of our analyses.

### Replication in the Third Cohort

We performed a replication study of the OAS3-R381S in patients from the 3^{rd} hospital (KK) during the same epidemic (2001-2003). The C allele showed less frequency in DSS group compared with DV patients without shock similar to previous results (8% versus 17% for C allele frequency and 17% versus 32% for CG+GG frequency). Although, this difference was not statistically significant because of small number of patients, higher significance association was obtained when combining all patients from the 3 hospitals (OR=0.48, 95 CI=0.31-0.75, P=7×10⁻⁴, for dominant model of the C allele).

### OAS3 and Type of Immune Response

We investigated further whether the effect of OAS3-R381S was depending on type of immune response to the virus. There was no significant association of the polymorphism and DSS in patients with predominant IgM immune response. However, significant and nearly significant association was obtained in patients with predominant IgG response in each hospital and combined (Chit=19.41, OR=0.27 95 CI=0.14-0.53, P<10-5) (Table VII).

TABLE VII
OAS3-R381S in IgM and IgG immune response

| | IgM response | | | | | IgG response | | | | |
|---|---|---|---|---|---|---|---|---|---|---|
| | Number | | Frequency (%) | | | Number | | Frequency (%) | | |
| Hospital and group | GG | CG + CC | GG | CG + CC | P | GG | CG + CC | GG | CG + CC | P |
| Siriraj | | | | | | | | | | |
| Non-shock DENV | 28 | 14 | 66.7 | 33.3 | | 126 | 59 | 68.1 | 31.9 | |
| DSS | 2 | 0 | 100 | 0 | 1 | 35 | 3 | 92.1 | 7.9 | 0.002 |
| Rama | | | | | | | | | | |
| Non-shock DV | 33 | 16 | 67.3 | 32.7 | | 128 | 53 | 70.7 | 29.3 | |
| DSS | 3 | 2 | 60 | 40 | 1 | 33 | 5 | 86.8 | 13.2 | 0.044 |
| Khon-Kaen | | | | | | | | | | |
| Non-shock DV | 17 | 4 | 80.9 | 19.1 | | 81 | 41 | 66.4 | 33.6 | |
| DSS | 0 | 1 | 0 | 100 | 0.23 | 20 | 3 | 87 | 13 | 0.052 |

### Association Study of Patients During 2004-2006 Epidemics

During 2004-2006, we obtained additional patients from previous 2 hospitals (RA and KK, Table IV). Association study of OAS3-R381S did not reveal a significant association with all hypotheses studied, however, significant association remained when these patients were added to the original case/control study (OR=0.48, 95 CI=0.31-0.75, P=7×10⁻⁴). This result prompted us to investigate the discordance of the results between the 2 periods.

One factor that could contribute to severity of dengue disease is a viral factor. We investigated prevalence of DV-4 serotypes during the 2001-2006 using data of patients from Siriraj Hospital and Khonkaen hospital of which we have data from 471 patients (Table VIII).

TABLE VIII
Dengue virus serotype

| DV serotype | | | |
|---|---|---|---|
| 1 | 2 | 3 | 4 |

A. Dengue serotype by year of study

| | | | | |
|---|---|---|---|---|
| Year | | | | |
| 2001 | 57 (46%) | 45 (36.3%) | 13 (10.5%) | 9 (7.2%) |
| 2002 | 52 (46.4%) | 50 (44.7%) | 3 (2.7%) | 7 (6.2%) |
| 2003 | 53 (53%) | 36 (36%) | 4 (4%) | 7 (7%) |
| 2004 | 5 (13.5%) | 20 (54%) | 0 (0%) | 12 (32.4%) |
| 2005 | 11 (30.6%) | 3 (8.3%) | 1 (2.8%) | 21 (58.3%) |
| 2006 | 33 (53.2%) | 3 (4.8%) | 1 (1.6%) | 25 (40.3%) |

B. Dengue serotype and clinical severity

| | | | | |
|---|---|---|---|---|
| Clinical | | | | |
| Diagnosis | | | | |
| DF | 93 | 46 | 10 | 39 |
| DHF1 | 32 | 32 | 3 | 8 |
| DHF2 | 50 | 40 | 7 | 19 |
| DSS | 25 | 35 | 1 | 8 |
| UC | 11 | 3 | 1 | 6 |

C. Dengue serotype and type of immune response

| | | | | |
|---|---|---|---|---|
| Type of | | | | |
| Immune | | | | |
| Response | | | | |
| IgM | 44 | 5 | 7 | 0 |
| predominant | | | | |
| % | 20.6 | 3 | 21.9 | 0 |
| IgG | 170 | 159 | 25 | 82 |
| predominant | | | | |
| % | 79.4 | 97 | 78.1 | 100 |

DV1 and DV2 serotypes were predominant during 2001-2003 while in 2004 DV1 decreased while epidemic of DV4 started in 2004 and was, then, predominant until 2006 (Table 5A). Severity of dengue disease was significantly different among different serotypes of the virus (P=0.012, Table 5B) with DV2 serotype having more severe cases than the other DV serotypes. Surprisingly, serotypes of the viruses were significantly different according to the type of immune response (Fisher exact P<10-4, Table 5C) with DV2 and DV4 showing with the predominant IgG. These results were similar to data from Ministry of Public Health. We hypothesized that the effect of OAS3-R381S could be specific to some viral serotypes. We found significant dominant protective effect of OAS3-R381S against DSS only with DV2 (OR=0.24, 95 CI=0.08-0.74, P=0.0045, Table IX).

TABLE IX
OAS3-R381S and Dengue serotype

| | Number | | Frequency (%) | | |
|---|---|---|---|---|---|
| | GG | CG + CC | GG | CG + CC | P |
| Serotype 1 | | | | | |
| non-shock DV | 125 | 57 | 68.7 | 31.3 | 0.32 |
| DSS | 21 | 6 | 77.8 | 22.2 | |
| Serotype 2 | | | | | |
| non-shock DV | 82 | 42 | 66.1 | 33.9 | 0.0045 |
| DSS | 32 | 4 | 88.9 | 11.1 | |
| Serotype 4 | | | | | |
| non-shock DV | 49 | 21 | 70 | 30 | 0.77 |
| DSS | 6 | 2 | 75 | 25 | |

Based on a systematic screening of OAS genes in DF/DHF/DSS patients and controls, we have shown association between polymorphisms of the OAS3 gene and the risk of shock (DSS), and the severity of the disease in patients with dengue virus infection. The variant (OAS3-R381S: rs2285933) is a G to C substitution 3^{rd} nucleotide of codon 381 of OAS3, that changes amino acid from arginine to serine. The C variant is associated with dominant protection against DSS comparing with non-shock dengue disease, with frequency ratio of 0.13:0.32 (OR=0.48, 95 CI=0.31-0.75, P=7×10⁴). Furthermore, significant association was found exclusively in patients with predominant IgG response (OR=0.46, 95 CI=0.28-0.74, P=8×10⁴) and in patients infected by DV serotype 2 (OR=0.24, 95 CI=0.08-0.74, P=0.0045).

These results support a functional role of the non-synonymous polymorphism OAS3-R381S and demonstrate a role for OAS3 in the dengue pathogenesis, and interaction between a variant of OAS3 and serotype of the virus.

Our finding may have important consequences for the prediction of disease severity in DV infected patients, and for the development of OAS3-based prophylaxis and therapy against infections by Dengue virus and other positive-sense single-stranded RNA viruses of major medical importance.

1-28. (canceled)

29. An isolated 2′-5′-oligoadenylate synthetase 3 protein comprising an amino acid sequence with at least 70% amino acid sequence identity to residues 1 to 1087 of SEQ ID NO: 2.

30. The isolated 2′-5′-oligoadenylate synthetase 3 protein of claim 29 comprising an amino acid sequence with at least 80% amino acid sequence identity to residues 1 to 1087 of SEQ ID NO: 2.

31. The isolated 2′-5′-oligoadenylate synthetase 3 protein of claim 29 wherein the isolated 2′-5′-oligoadenylate synthetase 3 protein is human.

32. The isolated 2′-5′-oligoadenylate synthetase 3 protein of claim 29, wherein the amino acid sequence contains a Serine at position 381 of SEQ ID NO: 2.

33. The isolated 2′-5′-oligoadenylate synthetase 3 protein of claim 32, wherein the amino acid sequence comprises SEQ ID NO: 2.

34. The isolated 2′-5′-oligoadenylate synthetase 3 protein of claim 32, wherein the amino acid sequence comprises SEQ ID NO: 7.

35. An isolated polynucleotide encoding the isolated 2′-5′-oligoadenylate synthetase 3 protein of claim 29.

36. The isolated polynucleotide of claim 35, wherein the nucleotide sequence comprises SEQ ID NO: 1.

37. The isolated polynucleotide of claim 35, wherein the nucleotide sequence comprises SEQ ID NO: 6.

38. An expression vector comprising the polynucleotide of claim 35.

39. A method of treating or preventing a positive-sense single-stranded RNA virus infection in a subject in need thereof comprising administering the isolated 2′-5′-oligoadenylate synthetase 3 protein of claim 29 or the isolated polynucleotide of claim 35.

40. The method of claim 39, wherein the positive-sense single-stranded RNA virus is from the Alphavirus genus.

41. The method of claim 40, wherein said Alphavirus is selected from the group consisting of: Chikungunya virus, Sindbis virus, Eastern equine encephalitis virus, Western equine encephalitis virus, Venezuelan equine encephalitis virus, Ross River virus, Semliki Forest virus, Barma Forest virus and O'nyong'nyong virus.

42. The method of claim 39, wherein the positive-sense single-stranded RNA virus is from the Flavivirus genus.

43. The method of claim 42, wherein said Flavivirus is selected from the group consisting of: Dengue virus, Japanese encephalitis virus, Kyasanur Forest disease virus, Murray Valley encephalitis virus, St. Louis encephalitis virus, Tick-borne encephalitis virus, West Nile encephalitis virus, Yellow fever virus and Omsk hemorrhagic fever virus.

44. The method of claim 39, wherein the positive-sense single-stranded RNA virus is Hepatitis C.

45. A pharmaceutical composition comprising the isolated 2′-5′-oligoadenylate synthetase 3 protein of claim 29 and a second active agent selected from the group consisting of: an antiviral, anti-inflammatory and immunomodulatory agent.

46. A method of treating or preventing a positive-sense single-stranded RNA virus infection in a subject in need thereof comprising administering the pharmaceutical composition of claim 45.

47. A method for evaluating the susceptibility of an individual to a positive-sense single-stranded RNA virus infection comprising detecting the presence of a polymorphism in the 2′-5′-oligoadenylate synthetase 3 gene in a nucleic acid sample obtained from said individual, wherein the presence of the polymorphism indicates susceptibility to a positive-sense single-stranded RNA virus infection.

48. The method of claim 47, wherein said the polymorphism is detected using an 2′-5′-oligoadenylate synthetase 3 specific oligonucleotide selected in the group consisting of SEQ ID NO: 11, 12, 62 to 86, 118 to 141, and 142.

49. The method of claim 47, wherein said polymorphism is selected in the group consisting of: Arg844 codon to a stop codon (R844X) and Arginine 381 codon to a Serine codon (R381S).

50. The method of claim 49, wherein the R844X mutation is detected by a PCR-RFLP assay comprising: (a) amplification of a nucleotide sequence from the nucleic acid sample using SEQ ID NO: 11 and 12, and (b) digesting the amplified nucleic acid sequence with BglII endonuclease, wherein the presence of two fragments of 115 bp and 68 bp indicates the presence of the R844X mutation.

51. The method of claim 49, wherein the R381S polymoprhism is associated with a dominant protection against the risk of dengue shock syndrome in dengue virus infected patients.

52. An isolated 2′-5′-oligoadenylate synthetase 3 protein comprising SEQ ID NO: 30.

53. An isolated 2′-5′-oligoadenylate synthetase protein fragment consisting of SEQ ID NO: 9.

54. An isolated polynucleotide selected in the group consisting of: (a) a nucleotide sequence encoding the 2′-5′-oligoadenylate synthetase 3 protein of SEQ ID NO: 7, (b) a nucleotide sequence comprising SEQ ID NO: 6, (c) a nucleotide sequence encoding the 2′-5′-oligoadenylate synthetase 3 protein SEQ ID NO: 30, (d) a nucleotide sequence comprising SEQ ID NO: 28, (e) a nucleotide sequence comprising SEQ ID NO: 29, (e) a nucleotide sequence encoding the 2′-5′-oligoadenylate synthetase 3 protein fragment SEQ ID NO: 9, and (f) a nucleotide sequence comprising SEQ ID NO: 8.

55. A recombinant vector comprising the isolated polynucleotide sequence of claim 54.

56. A host cell transfected with or transformed by the isolated polynucleotide of claim 54 or a recombinant vector of claim 55.

57. The host cell of claim 56, deposited Feb. 26, 2008, at the Collection Nationale de Cultures de Microorganismes, 25 rue du Docteur Roux, 75724 Paris Cedex 15, under accession number I-3927.

58. The host cell of claim 56, deposited 17 Apr. 2008, at the Collection Nationale de Cultures de Microorganismes, 25 rue du Docteur Roux, 75724 Paris Cedex 15, under accession number I-3968.

59. The host cell of claim 56, deposited May 15, 2009, at the Collection Nationale de Cultures de Microorganismes, 25 rue du Docteur Roux, 75724 Paris Cedex 15, under accession number I-4158.


SEQUENCE LISTING (142 sequences)

SEQ ID NO: 1 - DNA, 3264 bp, Homo sapiens
  CDS (1)..(3264)
atggacttgtacagcaccccggccgctgcgctggacaggttcgtggccagaaggctgcagccgcggaaggagttcgtagagaaggcgcggcgcgctctgggcgccctggccgctgccctgagggagcgcgggggccgcctcggtgctgctgccccgcgggtgctgaaaactgtcaagggaggctcctcgggccggggcacagctctcaagggtggctgtgattctgaacttgtcatcttcctcgactgcttcaagagctatgtggaccagagggcccgccgtgcagagatcctcagtgagatgcgggcatcgctggaatcctggtggcagaacccagtccctggtctgagactcacgtttcctgagcagagcgtgcctggggccctgcagttccgcctgacatccgtagatcttgaggactggatggatgttagcctggtgcctgccttcaatgtcctgggtcaggccggctccggcgtcaaacccaagccacaagtctactctaccctcctcaacagtggctgccaagggggcgagcatgcggcctgcttcacagagctgcggaggaactttgtgaacattcgcccagccaagttgaagaacctaatcttgctggtgaagcactggtaccaccaggtgtgcctacaggggttgtggaaggagacgctgcccccggtctatgccctggaattgctgaccatcttcgcctgggagcagggctgtaagaaggatgctttcagcctagccgaaggcctccgaactgtcctgggcctgatccaacagcatcagcacctgtgtgttttctggactgtcaactatggcttcgaggaccctgcagttgggcagttcttgcagcggcagcttaagagacccaggcctgtgatcctggacccagctgaccccacatgggacctggggaatggggcagcctggcactgggatttgctagcccaggaggcagcatcctgctatgaccacccatgctttctgagggggatgggggacccagtgcagtcttggaaggggccgggccttccacgtgctggatgctcaggtttgggccaccccatccagctagaccctaaccagaagacccctgaaaacagcaagagcctcaatgctgtgtacccaagagcagggagcaaacctccctcatgcccagctcctggccccactggggcagccagcatcgtcccctctgtgccgggaatggccttggacctgtctcagatccccaccaaggagctggaccgcttcatccaggaccacctgaagccgagcccccagttccaggagcaggtgaaaaaggccatcgacatcatcttgcgctgcctccatgagaactgtgttcacaaggcctcaagagtcagtaaagggggctcatttggccggggcacagacctaagggatggctgtgatgttgaactcatcatcttcctcaactgcttcacggactacaaggaccaggggccccgccgcgcagagatccttgatgagatgcgagcgcagctagaatcctggtggcaggaccaggtgcccagcctgagccttcagtttcctgagcagaatgtgcctgaggctctgcagttccagctggtgtccacagccctgaagagctggacggatgttagcctgctgcctgccttcgatgctgtggggcagctcagttctggcaccaaaccaaatccccaggtctactcgaggctcctcaccagtggctgccaggagggcgagcataaggcctgcttcgcagagctgcggaggaacttcatgaacattcgccctgtcaagctgaagaacctgattctgctggtgaagcactggtaccgccaggttgcggctcagaacaaaggaaaaggaccagcccctgcctctctgcccccagcctatgccctggagctcctcaccatctttgcctgggagcagggctgcaggcaggattgtttcaacatggcccaaggcttccggacggtgctggggctcgtgcaacagcatcagcagctctgtgtctactggacggtcaactatagcactgaggacccagccatgagaatgcaccttcttggccagcttcgaaaacccagacccctggtcctggaccccgctgatcccacctggaacgtgggccacggtagctgggagctgttggcccaggaagcagcagcgctggggatgcaggcctgctttctgagtagagacgggacatctgtgcagccctgggatgtgatgccagccctcctttaccaaaccccagctggggaccttgacaagttcatcagtgaatttctccagcccaaccgccagttcctggcccaggtgaacaaggccgttgataccatctgttcatttttgaaggaaaactgcttccggaattctcccatcaaagtgatcaaggtggtcaagggtggctcttcagccaaaggcacagctctgcgaggccgctcagatgccgacctcgtggtgttcctcagctgcttcagccagttcactgagcagggcaacaagcgggccgagatcatctccgagatccgagcccagctggaggcatgtcaacaggagcggcagttcgaggtcaagtttgaagtctccaaatgggagaatccccgcgtgctgagcttctcactgacatcccagacgatgctggaccagagtgtggactttgatgtgctgccagcctttgacgccctaggccagctggtctctggctccaggcccagctctcaagtctacgtcgacctcatccacagctacagcaatgcgggcgagtactccacctgcttcacagagctacaacgggacttcatcatctctcgccctaccaagctgaagagcctgatccggctggtgaagcactggtaccagcagtgtaccaagatctccaaggggagaggctccctacccccacagcacgggctggaactcctgactgtgtatgcctgggagcagggcgggaaggactcccagttcaacatggctgagggcttccgcacggtcctggagctggtcacccagtaccgccagctctgtatctactggaccatcaactacaacgccaaggacaagactgttggagacttcctgaaacagcagcttcagaagcccaggcctatcatcctggatccggctgacccgacaggcaacctgggccacaatgcccgctgggacctgctggccaaggaagctgcagcctgcacatctgccctgtgctgcatgggacggaatggcatccccatccagccatggccagtgaaggctgctgtgtga

SEQ ID NO: 2 - protein, 1087 aa, Homo sapiens
MDLYSTPAAALDRFVARRLQPRKEFVEKARRALGALAAALRERGGRLGAAAPRVLKTVKGGSSGRGTALKGGCDSELVIFLDCFKSYVDQRARRAEILSEMRASLESWWQNPVPGLRLTFPEQSVPGALQFRLTSVDLEDWMDVSLVPAFNVLGQAGSGVKPKPQVYSTLLNSGCQGGEHAACFTELRRNFVNIRPAKLKNLILLVKHWYHQVCLQGLWKETLPPVYALELLTIFAWEQGCKKDAFSLAEGLRTVLGLIQQHQHLCVFWTVNYGFEDPAVGQFLQRQLKRPRPVILDPADPTWDLGNGAAWHWDLLAQEAASCYDHPCFLRGMGDPVQSWKGPGLPRAGCSGLGHPIQLDPNQKTPENSKSLNAVYPRAGSKPPSCPAPGPTGAASIVPSVPGMALDLSQIPTKELDRFIQDHLKPSPQFQEQVKKAIDIILRCLHENCVHKASRVSKGGSFGRGTDLRDGCDVELIIFLNCFTDYKDQGPRRAEILDEMRAQLESWWQDQVPSLSLQFPEQNVPEALQFQLVSTALKSWTDVSLLPAFDAVGQLSSGTKPNPQVYSRLLTSGCQEGEHKACFAELRRNFMNIRPVKLKNLILLVKHWYRQVAAQNKGKGPAPASLPPAYALELLTIFAWEQGCRQDCFNMAQGFRTVLGLVQQHQQLCVYWTVNYSTEDPAMRMHLLGQLRKPRPLVLDPADPTWNVGHGSWELLAQEAAALGMQACFLSRDGTSVQPWDVMPALLYQTPAGDLDKFISEFLQPNRQFLAQVNKAVDTICSFLKENCFRNSPIKVIKVVKGGSSAKGTALRGRSDADLVVFLSCFSQFTEQGNKRAEIISEIRAQLEACQQERQFEVKFEVSKWENPRVLSFSLTSQTMLDQSVDFDVLPAFDALGQLVSGSRPSSQVYVDLIHSYSNAGEYSTCFTELQRDFIISRPTKLKSLIRLVKHWYQQCTKISKGRGSLPPQHGLELLTVYAWEQGGKDSQFNMAEGFRTVLELVTQYRQLCIYWTINYNAKDKTVGDFLKQQLQKPRPIILDPADPTGNLGHNARWDLLAKEAAACTSALCCMGRNGIPIQPWPVKAAV

SEQ ID NO: 3 - protein, 338 aa, artificial sequence
  OAS domain I
TPAAALDRFVARRLQPRKEFVEKARRALGALAAALRERGGRLGAAAPRVLKTVKGGSSGRGTALKGGCDSELVIFLDCFKSYVDQRARRAEILSEMRASLESWWQNPVPGLRLTFPEQSVPGALQFRLTSVDLEDWMDVSLVPAFNVLGQAGSGVKPKPQVYSTLLNSGCQGGEHAACFTELRRNFVNIRPAKLKNLILLVKHWYHQVCLQGLWKETLPPVYALELLTIFAWEQGCKKDAFSLAEGLRTVLGLIQQHQHLCVFWTVNYGFEDPAVGQFLQRQLKRPRPVILDPADPTWDLGNGAAWHWDLLAQEAASCYDHPCFLRGMGDPVQSWKGP

SEQ ID NO: 4 - protein, 332 aa, artificial sequence
  OAS domain II
IPTKELDRFIQDHLKPSPQFQEQVKKAIDIILRCLHENCVHKASRVSKGGSFGRGTDLRDGCDVELIIFLNCFTDYKDQGPRRAEILDEMRAQLESWWQDQVPSLSLQFPEQNVPEALQFQLVSTALKSWTDVSLLPAFDAVGQLSSGTKPNPQVYSRLLTSGCQEGEHKACFAELRRNFMNIRPVKLKNLILLVKHWYRQVAAQNKGKGPAPASLPPAYALELLTIFAWEQGCRQDCFNMAQGFRTVLGLVQQHQQLCVYWTVNYSTEDPAMRMHLLGQLRKPRPLVLDPADPTWNVGHGSWELLAQEAAALGMQACFLSRDGTSVQPWDV

SEQ ID NO: 5 - protein, 335 aa, artificial sequence
  OAS domain III
TPAGDLDKFISEFLQPNRQFLAQVNKAVDTICSFLKENCFRNSPIKVIKVVKGGSSAKGTALRGRSDADLVVFLSCFSQFTEQGNKRAEIISEIRAQLEACQQERQFEVKFEVSKWENPRVLSFSLTSQTMLDQSVDFDVLPAFDALGQLVSGSRPSSQVYVDLIHSYSNAGEYSTCFTELQRDFIISRPTKLKSLIRLVKHWYQQCTKISKGRGSLPPQHGLELLTVYAWEQGGKDSQFNMAEGFRTVLELVTQYRQLCIYWTINYNAKDKTVGDFLKQQLQKPRPIILDPADPTGNLGHNARWDLLAKEAAACTSALCCMGRNGIPIQPWPVK

SEQ ID NO: 6 - DNA, 3264 bp, artificial sequence
  human recombinant OAS3
atggacttgtacagcaccccggccgctgcgctggacaggttcgtggccagaaagctgcagccgcggaaggagttcgtagagaaggcgcggcgcgctctgggcgccctggccgctgctctgagggagcgcgggggccgcctcggtgctgctgccccgcgggtgctgaaaactgtcaagggaggctcctcgggccggggcacagctctcaagggtggctgtgattctgaacttgtcatcttcctcgactgcttcaagagctatgtggaccagagggcccgccgtgcagagatcctcagtgagatgcgggcatcgctggaatcctggtggcagaacccagtccctggtctgagactcacgtttcctgagcagagcgtgcctggggccctgcagttccgcctgacatccgtagatcttgaggactggatggatgttagcctggtgcctgccttcaatgtcctgggtcaggccggctccggcgtcaaacccaagccacaagtctactctaccctcctcaacagtggctgccaagggggcgagcatgcggcctgcttcacagagctgcggaggaactttgtgaacattcgcccagccaagttgaagaacctaatcttgctggtgaagcactggtaccaccaggtgtgcctacaggggttgtggaaggagacgctgcccccggtctatgccctggaattgctgaccatcttcgcctgggagcagggctgtaagaaggatgctttcagcctagccgaaggcctccgaactgtcctgggcctgatccaacagcatcagcacctgtgtgttttctggactgtcaactatggcttcgaggaccctgcagttgggcagttcttgcagcggcagcttaagagacccaggcctgtgatcctggacccagctgaccccacatgggacctggggaatggggcagcctggcactgggatttgctagcccaggaggcagcatcctgctatgaccacccatgctttctgagggggatgggggacccagtgcagtcttggaaggggccgggccttccacgtgctggatgctcaggtttgggccaccccatccagctagaccctaaccagaagacccctgaaaacagcaagagcctcaatgctgtgtacccaagagcagggagcaaacctccctcatgcccagctcctggccccactggggcagccagcatcgtcccctctgtgccgggaatggccttggacctgtctcagatccccaccaaggagctggaccgcttcatccaggaccacctgaagccgagcccccagttccaggagcaggtgaaaaaggccatcgacatcatcttgcgctgcctccatgagaactgtgttcacaaggcctcaagagtcagtaaagggggctcatttggccggggcacagacctaagggatggctgtgatgttgaactcatcatcttcctcaactgcttcacggactacaaggaccaggggccccgccgcgcagagatccttgatgagatgcgagcgcagctagaatcctggtggcaggaccaggtgcccagcctgagccttcagtttcctgagcagaatgtgcctgaggctctgcagttccagctggtgtccacagccctgaagagctggacggatgttagcctgctgcctgccttcgatgctgtggggcagctcagttctggcaccaaaccaaatccccaggtctactcgaggctcctcaccagtggctgccaggagggcgagcataaggcctgcttcgcagagctgcggaggaacttcatgaacattcgccctgtcaagctgaagaacctgattctgctggtgaagcactggtaccgccaggttgcggctcagaacaaaggaaaaggaccagcccctgcctctctgcccccagcctatgccctggagctcctcaccatctttgcctgggagcagggctgcaggcaggattgtttcaacatggcccaaggcttccggacggtgctggggctcgtgcaacagcatcagcagctctgtgtctactggacggtcaactatagcactgaggacccagccatgagaatgcaccttcttggccagcttcgaaaacccagacccctggtcctggaccccgctgatcccacctggaacgtgggccacggtagctgggagctgttggcccaggaagcagcagcgctggggatgcaggcctgctttctgagtagagacgggacatctgtgcagccctgggatgtgatgccagccctcctttaccaaaccccagctggggaccttgacaagttcatcagtgaatttctccagcccaaccgccagttcctggcccaggtgaacaaggccgttgataccatctgttcatttttgaaggaaaactgcttccggaattctcccatcaaagtgatcaaggtggtcaagggtggctcttcagccaaaggcacagctctgcgaggccgctcagatgccgacctcgtggtgttcctcagctgcttcagccagttcactgagcagggcaacaagcgggccgagatcatctccgagatccgagcccagctggaggcatgtcaacaggagcggcagttcgaggtcaagtttgaagtctccaaatcggagaatccccgcgtgctgagcttctcactgacatcccagacgatgctggaccagagtgtggactttgatgtgctgccagcctttgacgccctaggccagctggtctctggctccaggcccagctctcaagtctacgtcgacctcatccacagctacagcaatgcgggcgagtactccacctgcttcacagagctacaacgggacttcatcatctctcgccctaccaagctgaagagcctgatccggctggtgaagcactggtaccagcagtgtaccaagatctccaaggggagaggctccctacccccacagcacgggctggaactcctgactgtgtatgcctgggagcagggcgggaaggactcccagttcaacatggctgagggcttccgcacggtcctggagctggtcacccagtaccgccagctctgtatctactggaccatcaactacaacgccaaggacaagactgttggagacttcctgaaacagcagcttcagaagcccaggcctatcatcctggatccggctgacccgacaggcaacctgggccacaatgcccgctgggacctgctggccaaggaagctgcagcctgcacatctgccctgtgctgcatggggcggaatggcatccccatccagccatggccagtgaaggctgctgtgtga

SEQ ID NO: 7 - protein, 1087 aa, artificial sequence
  Synthetic Construct
MDLYSTPAAALDRFVARKLQPRKEFVEKARRALGALAAALRERGGRLGAAAPRVLKTVKGGSSGRGTALKGGCDSELVIFLDCFKSYVDQRARRAEILSEMRASLESWWQNPVPGLRLTFPEQSVPGALQFRLTSVDLEDWMDVSLVPAFNVLGQAGSGVKPKPQVYSTLLNSGCQGGEHAACFTELRRNFVNIRPAKLKNLILLVKHWYHQVCLQGLWKETLPPVYALELLTIFAWEQGCKKDAFSLAEGLRTVLGLIQQHQHLCVFWTVNYGFEDPAVGQFLQRQLKRPRPVILDPADPTWDLGNGAAWHWDLLAQEAASCYDHPCFLRGMGDPVQSWKGPGLPRAGCSGLGHPIQLDPNQKTPENSKSLNAVYPRAGSKPPSCPAPGPTGAASIVPSVPGMALDLSQIPTKELDRFIQDHLKPSPQFQEQVKKAIDIILRCLHENCVHKASRVSKGGSFGRGTDLRDGCDVELIIFLNCFTDYKDQGPRRAEILDEMRAQLESWWQDQVPSLSLQFPEQNVPEALQFQLVSTALKSWTDVSLLPAFDAVGQLSSGTKPNPQVYSRLLTSGCQEGEHKACFAELRRNFMNIRPVKLKNLILLVKHWYRQVAAQNKGKGPAPASLPPAYALELLTIFAWEQGCRQDCFNMAQGFRTVLGLVQQHQQLCVYWTVNYSTEDPAMRMHLLGQLRKPRPLVLDPADPTWNVGHGSWELLAQEAAALGMQACFLSRDGTSVQPWDVMPALLYQTPAGDLDKFISEFLQPNRQFLAQVNKAVDTICSFLKENCFRNSPIKVIKVVKGGSSAKGTALRGRSDADLVVFLSCFSQFTEQGNKRAEIISEIRAQLEACQQERQFEVKFEVSKSENPRVLSFSLTSQTMLDQSVDFDVLPAFDALGQLVSGSRPSSQVYVDLIHSYSNAGEYSTCFTELQRDFIISRPTKLKSLIRLVKHWYQQCTKISKGRGSLPPQHGLELLTVYAWEQGGKDSQFNMAEGFRTVLELVTQYRQLCIYWTINYNAKDKTVGDFLKQQLQKPRPIILDPADPTGNLGHNARWDLLAKEAAACTSALCCMGRNGIPIQPWPVKAAV

SEQ ID NO: 8 - DNA, 2532 bp, artificial sequence
  OAS3 truncated protein (OAS3 1-843 or OAS3 delta-C-term)
atggacttgtacagcaccccggccgctgcgctggacaggttcgtggccagaaagctgcagccgcggaaggagttcgtagagaaggcgcggcgcgctctgggcgccctggccgctgctctgagggagcgcgggggccgcctcggtgctgctgccccgcgggtgctgaaaactgtcaagggaggctcctcgggccggggcacagctctcaagggtggctgtgattctgaacttgtcatcttcctcgactgcttcaagagctatgtggaccagagggcccgccgtgcagagatcctcagtgagatgcgggcatcgctggaatcctggtggcagaacccagtccctggtctgagactcacgtttcctgagcagagcgtgcctggggccctgcagttccgcctgacatccgtagatcttgaggactggatggatgttagcctggtgcctgccttcaatgtcctgggtcaggccggctccggcgtcaaacccaagccacaagtctactctaccctcctcaacagtggctgccaagggggcgagcatgcggcctgcttcacagagctgcggaggaactttgtgaacattcgcccagccaagttgaagaacctaatcttgctggtgaagcactggtaccaccaggtgtgcctacaggggttgtggaaggagacgctgcccccggtctatgccctggaattgctgaccatcttcgcctgggagcagggctgtaagaaggatgctttcagcctagccgaaggcctccgaactgtcctgggcctgatccaacagcatcagcacctgtgtgttttctggactgtcaactatggcttcgaggaccctgcagttgggcagttcttgcagcggcagcttaagagacccaggcctgtgatcctggacccagctgaccccacatgggacctggggaatggggcagcctggcactgggatttgctagcccaggaggcagcatcctgctatgaccacccatgctttctgagggggatgggggacccagtgcagtcttggaaggggccgggccttccacgtgctggatgctcaggtttgggccaccccatccagctagaccctaaccagaagacccctgaaaacagcaagagcctcaatgctgtgtacccaagagcagggagcaaacctccctcatgcccagctcctggccccactggggcagccagcatcgtcccctctgtgccgggaatggccttggacctgtctcagatccccaccaaggagctggaccgcttcatccaggaccacctgaagccgagcccccagttccaggagcaggtgaaaaaggccatcgacatcatcttgcgctgcctccatgagaactgtgttcacaaggcctcaagagtcagtaaagggggctcatttggccggggcacagacctaagggatggctgtgatgttgaactcatcatcttcctcaactgcttcacggactacaaggaccaggggccccgccgcgcagagatccttgatgagatgcgagcgcagctagaatcctggtggcaggaccaggtgcccagcctgagccttcagtttcctgagcagaatgtgcctgaggctctgcagttccagctggtgtccacagccctgaagagctggacggatgttagcctgctgcctgccttcgatgctgtggggcagctcagttctggcaccaaaccaaatccccaggtctactcgaggctcctcaccagtggctgccaggagggcgagcataaggcctgcttcgcagagctgcggaggaacttcatgaacattcgccctgtcaagctgaagaacctgattctgctggtgaagcactggtaccgccaggttgcggctcagaacaaaggaaaaggaccagcccctgcctctctgcccccagcctatgccctggagctcctcaccatctttgcctgggagcagggctgcaggcaggattgtttcaacatggcccaaggcttccggacggtgctggggctcgtgcaacagcatcagcagctctgtgtctactggacggtcaactatagcactgaggacccagccatgagaatgcaccttcttggccagcttcgaaaacccagacccctggtcctggaccccgctgatcccacctggaacgtgggccacggtagctgggagctgttggcccaggaagcagcagcgctggggatgcaggcctgctttctgagtagagacgggacatctgtgcagccctgggatgtgatgccagccctcctttaccaaaccccagctggggaccttgacaagttcatcagtgaatttctccagcccaaccgccagttcctggcccaggtgaacaaggccgttgataccatctgttcatttttgaaggaaaactgcttccggaattctcccatcaaagtgatcaaggtggtcaagggtggctcttcagccaaaggcacagctctgcgaggccgctcagatgccgacctcgtggtgttcctcagctgcttcagccagttcactgagcagggcaacaagcgggccgagatcatctccgagatctga

SEQ ID NO: 9 - protein, 843 aa, artificial sequence
  Synthetic Construct
MDLYSTPAAALDRFVARKLQPRKEFVEKARRALGALAAALRERGGRLGAAAPRVLKTVKGGSSGRGTALKGGCDSELVIFLDCFKSYVDQRARRAEILSEMRASLESWWQNPVPGLRLTFPEQSVPGALQFRLTSVDLEDWMDVSLVPAFNVLGQAGSGVKPKPQVYSTLLNSGCQGGEHAACFTELRRNFVNIRPAKLKNLILLVKHWYHQVCLQGLWKETLPPVYALELLTIFAWEQGCKKDAFSLAEGLRTVLGLIQQHQHLCVFWTVNYGFEDPAVGQFLQRQLKRPRPVILDPADPTWDLGNGAAWHWDLLAQEAASCYDHPCFLRGMGDPVQSWKGPGLPRAGCSGLGHPIQLDPNQKTPENSKSLNAVYPRAGSKPPSCPAPGPTGAASIVPSVPGMALDLSQIPTKELDRFIQDHLKPSPQFQEQVKKAIDIILRCLHENCVHKASRVSKGGSFGRGTDLRDGCDVELIIFLNCFTDYKDQGPRRAEILDEMRAQLESWWQDQVPSLSLQFPEQNVPEALQFQLVSTALKSWTDVSLLPAFDAVGQLSSGTKPNPQVYSRLLTSGCQEGEHKACFAELRRNFMNIRPVKLKNLILLVKHWYRQVAAQNKGKGPAPASLPPAYALELLTIFAWEQGCRQDCFNMAQGFRTVLGLVQQHQQLCVYWTVNYSTEDPAMRMHLLGQLRKPRPLVLDPADPTWNVGHGSWELLAQEAAALGMQACFLSRDGTSVQPWDVMPALLYQTPAGDLDKFISEFLQPNRQFLAQVNKAVDTICSFLKENCFRNSPIKVIKVVKGGSSAKGTALRGRSDADLVVFLSCFSQFTEQGNKRAEIISEI

SEQ ID NO: 10 - protein, 10 aa, artificial sequence
  cmyc tag
EQKLISKEDL

SEQ ID NO: 11 - DNA, 20 bp, artificial sequence
  primer
gtggtgttcctcagctgctt

SEQ ID NO: 12 - DNA, 20 bp, artificial sequence
  primer
catcgtctgggatgtcagtg

SEQ ID NO: 13 - DNA, 38 bp, artificial sequence
  primer
attaatgcggccgcaacgaaaccagaaatccgaaggcc

SEQ ID NO: 14 - DNA, 78 bp, artificial sequence
  primer
aactaactatagtcaaaggtcctccttggagatgagcttctgttccacagcagccttcactggccatggctggatggg

SEQ ID NO: 15 - DNA, 44 bp, artificial sequence
  primer
attaatgcggccgcatggacttgtacagcaccccggccgctgcg

SEQ ID NO: 16 - DNA, 81 bp, artificial sequence
  primer
aactaactatagtcaaaggtcctccttggagatgagcttctgttcgatctcggagatgatctcggcccgccgcttgttgcc

SEQ ID NO: 17 - DNA, 21 bp, artificial sequence
  primer
cggacggtgctggggctcgtg

SEQ ID NO: 18 - DNA, 24 bp, artificial sequence
  primer
cttgtcttcgagtagaggcttctc

SEQ ID NO: 19 - DNA, 17 bp, artificial sequence
  primer
caccgccgcaactaccg

SEQ ID NO: 20 - DNA, 17 bp, artificial sequence
  primer
gattggtgaccgcggca

SEQ ID NO: 21 - DNA, 14 bp, artificial sequence
  primer
gggagccaaaaggg

SEQ ID NO: 22 - DNA, 14 bp, artificial sequence
  primer
ggggacacggaagg

SEQ ID NO: 23 - DNA, 3308 bp, artificial sequence
  OAS3 c-myc tag
gcggccgcatggacttgtacagcaccccggccgctgcgctggacaggttcgtggccagaaagctgcagccgcggaaggagttcgtagagaaggcgcggcgcgctctgggcgccctggccgctgctctgagggagcgcgggggccgcctcggtgctgctgccccgcgggtgctgaaaactgtcaagggaggctcctcgggccggggcacagctctcaagggtggctgtgattctgaacttgtcatcttcctcgactgcttcaagagctatgtggaccagagggcccgccgtgcagagatcctcagtgagatgcgggcatcgctggaatcctggtggcagaacccagtccctggtctgagactcacgtttcctgagcagagcgtgcctggggccctgcagttccgcctgacatccgtagatcttgaggactggatggatgttagcctggtgcctgccttcaatgtcctgggtcaggccggctccggcgtcaaacccaagccacaagtctactctaccctcctcaacagtggctgccaagggggcgagcatgcggcctgcttcacagagctgcggaggaactttgtgaacattcgcccagccaagttgaagaacctaatcttgctggtgaagcactggtaccaccaggtgtgcctacaggggttgtggaaggagacgctgcccccggtctatgccctggaattgctgaccatcttcgcctgggagcagggctgtaagaaggatgctttcagcctagccgaaggcctccgaactgtcctgggcctgatccaacagcatcagcacctgtgtgttttctggactgtcaactatggcttcgaggaccctgcagttgggcagttcttgcagcggcagcttaagagacccaggcctgtgatcctggacccagctgaccccacatgggacctggggaatggggcagcctggcactgggatttgctagcccaggaggcagcatcctgctatgaccacccatgctttctgagggggatgggggacccagtgcagtcttggaaggggccgggccttccacgtgctggatgctcaggtttgggccaccccatccagctagaccctaaccagaagacccctgaaaacagcaagagcctcaatgctgtgtacccaagagcagggagcaaacctccctcatgcccagctcctggccccactggggcagccagcatcgtcccctctgtgccgggaatggccttggacctgtctcagatccccaccaaggagctggaccgcttcatccaggaccacctgaagccgagcccccagttccaggagcaggtgaaaaaggccatcgacatcatcttgcgctgcctccatgagaactgtgttcacaaggcctcaagagtcagtaaagggggctcatttggccggggcacagacctaagggatggctgtgatgttgaactcatcatcttcctcaactgcttcacggactacaaggaccaggggccccgccgcgcagagatccttgatgagatgcgagcgcagctagaatcctggtggcaggaccaggtgcccagcctgagccttcagtttcctgagcagaatgtgcctgaggctctgcagttccagctggtgtccacagccctgaagagctggacggatgttagcctgctgcctgccttcgatgctgtggggcagctcagttctggcaccaaaccaaatccccaggtctactcgaggctcctcaccagtggctgccaggagggcgagcataaggcctgcttcgcagagctgcggaggaacttcatgaacattcgccctgtcaagctgaagaacctgattctgctggtgaagcactggtaccgccaggttgcggctcagaacaaaggaaaaggaccagcccctgcctctctgcccccagcctatgccctggagctcctcaccatctttgcctgggagcagggctgcaggcaggattgtttcaacatggcccaaggcttccggacggtgctggggctcgtgcaacagcatcagcagctctgtgtctactggacggtcaactatagcactgaggacccagccatgagaatgcaccttcttggccagcttcgaaaacccagacccctggtcctggaccccgctgatcccacctggaacgtgggccacggtagctgggagctgttggcccaggaagcagcagcgctggggatgcaggcctgctttctgagtagagacgggacatctgtgcagccctgggatgtgatgccagccctcctttaccaaaccccagctggggaccttgacaagttcatcagtgaatttctccagcccaaccgccagttcctggcccaggtgaacaaggccgttgataccatctgttcatttttgaaggaaaactgcttccggaattctcccatcaaagtgatcaaggtggtcaagggtggctcttcagccaaaggcacagctctgcgaggccgctcagatgccgacctcgtggtgttcctcagctgcttcagccagttcactgagcagggcaacaagcgggccgagatcatctccgagatccgagcccagctggaggcatgtcaacaggagcggcagttcgaggtcaagtttgaagtctccaaatcggagaatccccgcgtgctgagcttctcactgacatcccagacgatgctggaccagagtgtggactttgatgtgctgccagcctttgacgccctaggccagctggtctctggctccaggcccagctctcaagtctacgtcgacctcatccacagctacagcaatgcgggcgagtactccacctgcttcacagagctacaacgggacttcatcatctctcgccctaccaagctgaagagcctgatccggctggtgaagcactggtaccagcagtgtaccaagatctccaaggggagaggctccctacccccacagcacgggctggaactcctgactgtgtatgcctgggagcagggcgggaaggactcccagttcaacatggctgagggcttccgcacggtcctggagctggtcacccagtaccgccagctctgtatctactggaccatcaactacaacgccaaggacaagactgttggagacttcctgaaacagcagcttcagaagcccaggcctatcatcctggatccggctgacccgacaggcaacctgggccacaatgcccgctgggacctgctggccaaggaagctgcagcctgcacatctgccctgtgctgcatggggcggaatggcatccccatccagccatggccagtgaaggctgctgtggaacagaagctcatctccaaggaggacctttgactatag

SEQ ID NO: 24 - protein, 1097 aa, artificial sequence
  Synthetic Construct
MDLYSTPAAALDRFVARKLQPRKEFVEKARRALGALAAALRERGGRLGAAAPRVLKTVKGGSSGRGTALKGGCDSELVIFLDCFKSYVDQRARRAEILSEMRASLESWWQNPVPGLRLTFPEQSVPGALQFRLTSVDLEDWMDVSLVPAFNVLGQAGSGVKPKPQVYSTLLNSGCQGGEHAACFTELRRNFVNIRPAKLKNLILLVKHWYHQVCLQGLWKETLPPVYALELLTIFAWEQGCKKDAFSLAEGLRTVLGLIQQHQHLCVFWTVNYGFEDPAVGQFLQRQLKRPRPVILDPADPTWDLGNGAAWHWDLLAQEAASCYDHPCFLRGMGDPVQSWKGPGLPRAGCSGLGHPIQLDPNQKTPENSKSLNAVYPRAGSKPPSCPAPGPTGAASIVPSVPGMALDLSQIPTKELDRFIQDHLKPSPQFQEQVKKAIDIILRCLHENCVHKASRVSKGGSFGRGTDLRDGCDVELIIFLNCFTDYKDQGPRRAEILDEMRAQLESWWQDQVPSLSLQFPEQNVPEALQFQLVSTALKSWTDVSLLPAFDAVGQLSSGTKPNPQVYSRLLTSGCQEGEHKACFAELRRNFMNIRPVKLKNLILLVKHWYRQVAAQNKGKGPAPASLPPAYALELLTIFAWEQGCRQDCFNMAQGFRTVLGLVQQHQQLCVYWTVNYSTEDPAMRMHLLGQLRKPRPLVLDPADPTWNVGHGSWELLAQEAAALGMQACFLSRDGTSVQPWDVMPALLYQTPAGDLDKFISEFLQPNRQFLAQVNKAVDTICSFLKENCFRNSPIKVIKVVKGGSSAKGTALRGRSDADLVVFLSCFSQFTEQGNKRAEIISEIRAQLEACQQERQFEVKFEVSKSENPRVLSFSLTSQTMLDQSVDFDVLPAFDALGQLVSGSRPSSQVYVDLIHSYSNAGEYSTCFTELQRDFIISRPTKLKSLIRLVKHWYQQCTKISKGRGSLPPQHGLELLTVYAWEQGGKDSQFNMAEGFRTVLELVTQYRQLCIYWTINYNAKDKTVGDFLKQQLQKPRPIILDPADPTGNLGHNARWDLLAKEAAACTSALCCMGRNGIPIQPWPVKAAVEQKLISKEDL

SEQ ID NO: 25 - DNA, 2576 bp, artificial sequence
  OAS3 truncated protein c-myc tag
gcggccgcatggacttgtacagcaccccggccgctgcgctggacaggttcgtggccagaaagctgcagccgcggaaggagttcgtagagaaggcgcggcgcgctctgggcgccctggccgctgctctgagggagcgcgggggccgcctcggtgctgctgccccgcgggtgctgaaaactgtcaagggaggctcctcgggccggggcacagctctcaagggtggctgtgattctgaacttgtcatcttcctcgactgcttcaagagctatgtggaccagagggcccgccgtgcagagatcctcagtgagatgcgggcatcgctggaatcctggtggcagaacccagtccctggtctgagactcacgtttcctgagcagagcgtgcctggggccctgcagttccgcctgacatccgtagatcttgaggactggatggatgttagcctggtgcctgccttcaatgtcctgggtcaggccggctccggcgtcaaacccaagccacaagtctactctaccctcctcaacagtggctgccaagggggcgagcatgcggcctgcttcacagagctgcggaggaactttgtgaacattcgcccagccaagttgaagaacctaatcttgctggtgaagcactggtaccaccaggtgtgcctacaggggttgtggaaggagacgctgcccccggtctatgccctggaattgctgaccatcttcgcctgggagcagggctgtaagaaggatgctttcagcctagccgaaggcctccgaactgtcctgggcctgatccaacagcatcagcacctgtgtgttttctggactgtcaactatggcttcgaggaccctgcagttgggcagttcttgcagcggcagcttaagagacccaggcctgtgatcctggacccagctgaccccacatgggacctggggaatggggcagcctggcactgggatttgctagcccaggaggcagcatcctgctatgaccacccatgctttctgagggggatgggggacccagtgcagtcttggaaggggccgggccttccacgtgctggatgctcaggtttgggccaccccatccagctagaccctaaccagaagacccctgaaaacagcaagagcctcaatgctgtgtacccaagagcagggagcaaacctccctcatgcccagctcctggccccactggggcagccagcatcgtcccctctgtgccgggaatggccttggacctgtctcagatccccaccaaggagctggaccgcttcatccaggaccacctgaagccgagcccccagttccaggagcaggtgaaaaaggccatcgacatcatcttgcgctgcctccatgagaactgtgttcacaaggcctcaagagtcagtaaagggggctcatttggccggggcacagacctaagggatggctgtgatgttgaactcatcatcttcctcaactgcttcacggactacaaggaccaggggccccgccgcgcagagatccttgatgagatgcgagcgcagctagaatcctggtggcaggaccaggtgcccagcctgagccttcagtttcctgagcagaatgtgcctgaggctctgcagttccagctggtgtccacagccctgaagagctggacggatgttagcctgctgcctgccttcgatgctgtggggcagctcagttctggcaccaaaccaaatccccaggtctactcgaggctcctcaccagtggctgccaggagggcgagcataaggcctgcttcgcagagctgcggaggaacttcatgaacattcgccctgtcaagctgaagaacctgattctgctggtgaagcactggtaccgccaggttgcggctcagaacaaaggaaaaggaccagcccctgcctctctgcccccagcctatgccctggagctcctcaccatctttgcctgggagcagggctgcaggcaggattgtttcaacatggcccaaggcttccggacggtgctggggctcgtgcaacagcatcagcagctctgtgtctactggacggtcaactatagcactgaggacccagccatgagaatgcaccttcttggccagcttcgaaaacccagacccctggtcctggaccccgctgatcccacctggaacgtgggccacggtagctgggagctgttggcccaggaagcagcagcgctggggatgcaggcctgctttctgagtagagacgggacatctgtgcagccctgggatgtgatgccagccctcctttaccaaaccccagctggggaccttgacaagttcatcagtgaatttctccagcccaaccgccagttcctggcccaggtgaacaaggccgttgataccatctgttcatttttgaaggaaaactgcttccggaattctcccatcaaagtgatcaaggtggtcaagggtggctcttcagccaaaggcacagctctgcgaggccgctcagatgccgacctcgtggtgttcctcagctgcttcagccagttcactgagcagggcaacaagcgggccgagatcatctccgagatcgaacagaagctcatctccaaggaggacctttgactatag

SEQ ID NO: 26 - protein, 853 aa, artificial sequence
  Synthetic Construct
MDLYSTPAAALDRFVARKLQPRKEFVEKARRALGALAAALRERGGRLGAAAPRVLKTVKGGSSGRGTALKGGCDSELVIFLDCFKSYVDQRARRAEILSEMRASLESWWQNPVPGLRLTFPEQSVPGALQFRLTSVDLEDWMDVSLVPAFNVLGQAGSGVKPKPQVYSTLLNSGCQGGEHAACFTELRRNFVNIRPAKLKNLILLVKHWYHQVCLQGLWKETLPPVYALELLTIFAWEQGCKKDAFSLAEGLRTVLGLIQQHQHLCVFWTVNYGFEDPAVGQFLQRQLKRPRPVILDPADPTWDLGNGAAWHWDLLAQEAASCYDHPCFLRGMGDPVQSWKGPGLPRAGCSGLGHPIQLDPNQKTPENSKSLNAVYPRAGSKPPSCPAPGPTGAASIVPSVPGMALDLSQIPTKELDRFIQDHLKPSPQFQEQVKKAIDIILRCLHENCVHKASRVSKGGSFGRGTDLRDGCDVELIIFLNCFTDYKDQGPRRAEILDEMRAQLESWWQDQVPSLSLQFPEQNVPEALQFQLVSTALKSWTDVSLLPAFDAVGQLSSGTKPNPQVYSRLLTSGCQEGEHKACFAELRRNFMNIRPVKLKNLILLVKHWYRQVAAQNKGKGPAPASLPPAYALELLTIFAWEQGCRQDCFNMAQGFRTVLGLVQQHQQLCVYWTVNYSTEDPAMRMHLLGQLRKPRPLVLDPADPTWNVGHGSWELLAQEAAALGMQACFLSRDGTSVQPWDVMPALLYQTPAGDLDKFISEFLQPNRQFLAQVNKAVDTICSFLKENCFRNSPIKVIKVVKGGSSAKGTALRGRSDADLVVFLSCFSQFTEQGNKRAEIISEIEQKLISKEDL

SEQ ID NO: 27 - DNA, 6646 bp, Homo sapiens
gttcggagagccgggcgggaaaacgaaaccagaaatccgaaggccgcgccagagccctgcttccccttgcacctgcgccgggcggccatggacttgtacagcaccccggccgctgcgctggacaggttcgtggccagaaggctgcagccgcggaaggagttcgtagagaaggcgcggcgcgctctgggcgccctggccgctgccctgagggagcgcgggggccgcctcggtgctgctgccccgcgggtgctgaaaactgtcaagggaggctcctcgggccggggcacagctctcaagggtggctgtgattctgaacttgtcatcttcctcgactgcttcaagagctatgtggaccagagggcccgccgtgcagagatcctcagtgagatgcgggcatcgctggaatcctggtggcagaacccagtccctggtctgagactcacgtttcctgagcagagcgtgcctggggccctgcagttccgcctgacatccgtagatcttgaggactggatggatgttagcctggtgcctgccttcaatgtcctgggtcaggccggctccggcgtcaaacccaagccacaagtctactctaccctcctcaacagtggctgccaagggggcgagcatgcggcctgcttcacagagctgcggaggaactttgtgaacattcgcccagccaagttgaagaacctaatcttgctggtgaagcactggtaccaccaggtgtgcctacaggggttgtggaaggagacgctgcccccggtctatgccctggaattgctgaccatcttcgcctgggagcagggctgtaagaaggatgctttcagcctagccgaaggcctccgaactgtcctgggcctgatccaacagcatcagcacctgtgtgttttctggactgtcaactatggcttcgaggaccctgcagttgggcagttcttgcagcggcagcttaagagacccaggcctgtgatcctggacccagctgaccccacatgggacctggggaatggggcagcctggcactgggatttgctagcccaggaggcagcatcctgctatgaccacccatgctttctgagggggatgggggacccagtgcagtcttggaaggggccgggccttccacgtgctggatgctcaggtttgggccaccccatccagctagaccctaaccagaagacccctgaaaacagcaagagcctcaatgctgtgtacccaagagcagggagcaaacctccctcatgcccagctcctggccccactggggcagccagcatcgtcccctctgtgccgggaatggccttggacctgtctcagatccccaccaaggagctggaccgcttcatccaggaccacctgaagccgagcccccagttccaggagcaggtgaaaaaggccatcgacatcatcttgcgctgcctccatgagaactgtgttcacaaggcctcaagagtcagtaaagggggctcatttggccggggcacagacctaagggatggctgtgatgttgaactcatcatcttcctcaactgcttcacggactacaaggaccaggggccccgccgcgcagagatccttgatgagatgcgagcgcagctagaatcctggtggcaggaccaggtgcccagcctgagccttcagtttcctgagcagaatgtgcctgaggctctgcagttccagctggtgtccacagccctgaagagctggacggatgttagcctgctgcctgccttcgatgctgtggggcagctcagttctggcaccaaaccaaatccccaggtctactcgaggctcctcaccagtggctgccaggagggcgagcataaggcctgcttcgcagagctgcggaggaacttcatgaacattcgccctgtcaagctgaagaacctgattctgctggtgaagcactggtaccgccaggttgcggctcagaacaaaggaaaaggaccagcccctgcctctctgcccccagcctatgccctggagctcctcaccatctttgcctgggagcagggctgcaggcaggattgtttcaacatggcccaaggcttccggacggtgctggggctcgtgcaacagcatcagcagctctgtgtctactggacggtcaactatagcactgaggacccagccatgagaatgcaccttcttggccagcttcgaaaacccagacccctggtcctggaccccgctgatcccacctggaacgtgggccacggtagctgggagctgttggcccaggaagcagcagcgctggggatgcaggcctgctttctgagtagagacgggacatctgtgcagccctgggatgtgatgccagccctcctttaccaaaccccagctggggaccttgacaagttcatcagtgaatttctccagcccaaccgccagttcctggcccaggtgaacaaggccgttgataccatctgttcatttttgaaggaaaactgcttccggaattctcccatcaaagtgatcaaggtggtcaagggtggctcttcagccaaaggcacagctctgcgaggccgctcagatgccgacctcgtggtgttcctcagctgcttcagccagttcactgagcagggcaacaagcgggccgagatcatctccgagatccgagcccagctggaggcatgtcaacaggagcggcagttcgaggtcaagtttgaagtctccaaatgggagaatccccgcgtgctgagcttctcactgacatcccagacgatgctggaccagagtgtggactttgatgtgctgccagcctttgacgccctaggccagctggtctctggctccaggcccagctctcaagtctacgtcgacctcatccacagctacagcaatgcgggcgagtactccacctgcttcacagagctacaacgggacttcatcatctctcgccctaccaagctgaagagcctgatccggctggtgaagcactggtaccagcagtgtaccaagatctccaaggggagaggctccctacccccacagcacgggctggaactcctgactgtgtatgcctgggagcagggcgggaaggactcccagttcaacatggctgagggcttccgcacggtcctggagctggtcacccagtaccgccagctctgtatctactggaccatcaactacaacgccaaggacaagactgttggagacttcctgaaacagcagcttcagaagcccaggcctatcatcctggatccggctgacccgacaggcaacctgggccacaatgcccgctgggacctgctggccaaggaagctgcagcctgcacatctgccctgtgctgcatgggacggaatggcatccccatccagccatggccagtgaaggctgctgtgtgaagttgagaaaatcagcggtcctactggatgaagagaagatggacaccagccctcagcatgaggaaattcagggtcccctaccagatgagagagattgtgtacatgtgtgtgtgagcacatgtgtgcatgtgtgtgcacacgtgtgcatgtgtgtgttttagtgaatctgctctcccagctcacacactcccctgcctcccatggcttacacactaggatccagactccatggtttgacaccagcctgcgtttgcagcttctctgtcacttccatgactctatcctcataccaccactgctgcttcccacccagctgagaatgccccctcctccctgactcctctctgcccatgcaaattagctcacatctttcctcctgctgcaatccatcccttcctcccattggcctctccttgccaaatctaaatagtttatatagggatggcagagagttcccatctcatctgtcagccacagtcatttggtactggctacctggagccttatcttctgaagggttttaaagaatggccaattagctgagaagaattatctaatcaattagtgatgtctgccatggatgcagtagaggaaagtggtggtacaagtgccatgattgattagcaatgtctgcactggatacggaaaaaagaaggtgcttgcaggtttacagtgtatatgtgggctattgaagagccctctgagctcggttgctagcaggagagcatgcccatattggcttactttgtctgccacagacacagacagagggagttgggacatgcatgctatggggaccctcttgttggacacctaattggatgcctcttcatgagaggcctccttttcttcaccttttatgctgcactcctcccctagtttacacatcttgatgctgtggctcagtttgccttcctgaatttttattgggtccctgttttctctcctaacatgctgagattctgcatccccacagcctaaactgagccagtggccaaacaaccgtgctcagcctgtttctctctgccctctagagcaaggcccaccaggtccatccaggaggctctcctgacctcaagtccaacaacagtgtccacactagtcaaggttcagcccagaaaacagaaagcactctaggaatcttaggcagaaagggattttatctaaatcactggaaaggctggaggagcagaaggcagaggccaccactggactattggtttcaatattagaccactgtagccgaatcagaggccagagagcagccactgctactgctaatgccaccactacccctgccatcactgccccacatggacaaaactggagtcgagacctaggttagattcctgcaaccacaaacatccatcagggatggccagctgccagagctgcgggaagacggatcccacctccctttcttagcagaatctaaattacagccagacctctggctgcagaggagtctgagacatgtatgattgaatgggtgccaagtgccagggggcggagtccccagcagatgcatcctggccatctgttgcgtggatgagggagtgggtctatctcagaggaaggaacaggaaacaaagaaaggaagccactgaacatcccttctctgctccacaggagtgccttagacagcctgactctccacaaaccactgttaaaacttacctgctaggaatgctagattgaatgggatgggaagagccttccctcattattgtcattcttggagagaggtgagcaaccaagggaagctcctctgattcacctagaacctgttctctgccgtctttggctcagcctacagagactagagtaggtgaagggacagaggacagggcttctaatacctgtgccatattgacagcctccatccctgtcccccatcttggtgctgaaccaacgctaagggcaccttcttagactcacctcatcgatactgcctggtaatccaaagctagaactctcaggaccccaaactccacctcttggattggccctggctgctgccacacacatatccaagagctcagggccagttctggtgggcagcagagacctgctctgccaagttgtccagcagcagagtggccctggcctgggcatcacaagccagtgatgctcctgggaagaccaggtggcaggtcgcagttgggtaccttccattcccaccacacagactctgggcctccccgcaaaatggctccagaattagagtaattatgagatggtgggaaccagagcaactcaggtgcatgatacaaggagaggttgtcatctgggtagggcagagaggagggcttgctcatctgaacaggggtgtatttcattccaggccctcagtctttggcaatggccaccctggtgttggcatattggccccactgtaacttttgggggcttcccggtctagccacaccctcggatggaaagacttgactgcataaagatgtcagttctccctgagttgattgataggcttaatggtcaccctaaaaacacccacatatgcttttcgatggaaccaggtaagttgacgctaaagttcttatggaaaaatacacacgcaatagctaggaaaacacagggaaagaagagttctgagcagggcctagtcttagccaatattaaaacatactatgaagcctctgatacttaaacagcatggcgctggtacgtaaatagaccaatgcagttaggtggctctttccaagactctggggaaaaaagtagtaaaaagctaaatgcaatcaatcagcaattgaaagctaagtgagagagccagagggcctccttggtggtaaaagagggttgcatttcttgcagccagaaggcagagaaagtgaagaccaagtccagaactgaatcctaagaaatgcaggactgcaaagaaattggtgtgtgtgtgtgtgtgtgtgtgtgtgtgtgtgtttaatttttaaaaagtttttattgagatacaagtcaataccataaagctctcacccttctaaagtgtacaattcagtggtgtgagtatattcataagatttatacttggtgtctattcataagacttatatccagcatattcataactagagccatatcacagatgcattcatcataataattccagacattttcatcaccctaaaaggaaaccctgaaacccattagcagtcattccccattcctccaacccattctctccctaatccctagaaaccaccaatctgctgtgtatttcatctattgccaacatttcatataaatggcatcatacaaaaaaaaaaaaaaaaaaaaa

SEQ ID NO: 28 - DNA, 6646 bp, Homo sapiens
gttcggagagccgggcgggaaaacgaaaccagaaatccgaaggccgcgccagagccctgcttccccttgcacctgcgccgggcggccatggacttgtacagcaccccggccgctgcgctggacaggttcgtggccagaaggctgcagccgcggaaggagttcgtagagaaggcgcggcgcgctctgggcgccctggccgctgccctgagggagcgcgggggccgcctcggtgctgctgccccgcgggtgctgaaaactgtcaagggaggctcctcgggccggggcacagctctcaagggtggctgtgattctgaacttgtcatcttcctcgactgcttcaagagctatgtggaccagagggcccgccgtgcagagatcctcagtgagatgcgggcatcgctggaatcctggtggcagaacccagtccctggtctgagactcacgtttcctgagcagagcgtgcctggggccctgcagttccgcctgacatccgtagatcttgaggactggatggatgttagcctggtgcctgccttcaatgtcctgggtcaggccggctccggcgtcaaacccaagccacaagtctactctaccctcctcaacagtggctgccaagggggcgagcatgcggcctgcttcacagagctgcggaggaactttgtgaacattcgcccagccaagttgaagaacctaatcttgctggtgaagcactggtaccaccaggtgtgcctacaggggttgtggaaggagacgctgcccccggtctatgccctggaattgctgaccatcttcgcctgggagcagggctgtaagaaggatgctttcagcctagccgaaggcctccgaactgtcctgggcctgatccaacagcatcagcacctgtgtgttttctggactgtcaactatggcttcgaggaccctgcagttgggcagttcttgcagcggcagcttaagagacccaggcctgtgatcctggacccagctgaccccacatgggacctggggaatggggcagcctggcactgggatttgctagcccaggaggcagcatcctgctatgaccacccatgctttctgagggggatgggggacccagtgcagtcttggaaggggccgggccttccacgtgctggatgctcaggtttgggccaccccatccagctagaccctaaccagaagacccctgaaaacagcaagagcctcaatgctgtgtacccaagagcagggaggaaacctccctcatgcccagctcctggccccactggggcagccagcatcgtcccctctgtgccgggaatggccttggacctgtctcagatccccaccaaggagctggaccgcttcatccaggaccacctgaagccgagcccccagttccaggagcaggtgaaaaaggccatcgacatcatcttgcgctgcctccatgagaactgtgttcacaaggcctcaagagtcagtaaagggggctcatttggccggggcacagacctaagggatggctgtgatgttgaactcatcatcttcctcaactgcttcacggactacaaggaccaggggccccgccgcgcagagatccttgatgagatgcgagcgcagctagaatcctggtggcaggaccaggtgcccagcctgagccttcagtttcctgagcagaatgtgcctgaggctctgcagttccagctggtgtccacagccctgaagagctggacggatgttagcctgctgcctgccttcgatgctgtggggcagctcagttctggcaccaaaccaaatccccaggtctactcgaggctcctcaccagtggctgccaggagggcgagcataaggcctgcttcgcagagctgcggaggaacttcatgaacattcgccctgtcaagctgaagaacctgattctgctggtgaagcactggtaccgccaggttgcggctcagaacaaaggaaaaggaccagcccctgcctctctgcccccagcctatgccctggagctcctcaccatctttgcctgggagcagggctgcaggcaggattgtttcaacatggcccaaggcttccggacggtgctggggctcgtgcaacagcatcagcagctctgtgtctactggacggtcaactatagcactgaggacccagccatgagaatgcaccttcttggccagcttcgaaaacccagacccctggtcctggaccccgctgatcccacctggaacgtgggccacggtagctgggagctgttggcccaggaagcagcagcgctggggatgcaggcctgctttctgagtagagacgggacatctgtgcagccctgggatgtgatgccagccctcctttaccaaaccccagctggggaccttgacaagttcatcagtgaatttctccagcccaaccgccagttcctggcccaggtgaacaaggccgttgataccatctgttcatttttgaaggaaaactgcttccggaattctcccatcaaagtgatcaaggtggtcaagggtggctcttcagccaaaggcacagctctgcgaggccgctcagatgccgacctcgtggtgttcctcagctgcttcagccagttcactgagcagggcaacaagcgggccgagatcatctccgagatccgagcccagctggaggcatgtcaacaggagcggcagttcgaggtcaagtttgaagtctccaaatgggagaatccccgcgtgctgagcttctcactgacatcccagacgatgctggaccagagtgtggactttgatgtgctgccagcctttgacgccctaggccagctggtctctggctccaggcccagctctcaagtctacgtcgacctcatccacagctacagcaatgcgggcgagtactccacctgcttcacagagctacaacgggacttcatcatctctcgccctaccaagctgaagagcctgatccggctggtgaagcactggtaccagcagtgtaccaagatctccaaggggagaggctccctacccccacagcacgggctggaactcctgactgtgtatgcctgggagcagggcgggaaggactcccagttcaacatggctgagggcttccgcacggtcctggagctggtcacccagtaccgccagctctgtatctactggaccatcaactacaacgccaaggacaagactgttggagacttcctgaaacagcagcttcagaagcccaggcctatcatcctggatccggctgacccgacaggcaacctgggccacaatgcccgctgggacctgctggccaaggaagctgcagcctgcacatctgccctgtgctgcatgggacggaatggcatccccatccagccatggccagtgaaggctgctgtgtgaagttgagaaaatcagcggtcctactggatgaagagaagatggacaccagccctcagcatgaggaaattcagggtcccctaccagatgagagagattgtgtacatgtgtgtgtgagcacatgtgtgcatgtgtgtgcacacgtgtgcatgtgtgtgttttagtgaatctgctctcccagctcacacactcccctgcctcccatggcttacacactaggatccagactccatggtttgacaccagcctgcgtttgcagcttctctgtcacttccatgactctatcctcataccaccactgctgcttcccacccagctgagaatgccccctcctccctgactcctctctgcccatgcaaattagctcacatctttcctcctgctgcaatccatcccttcctcccattggcctctccttgccaaatctaaatagtttatatagggatggcagagagttcccatctcatctgtcagccacagtcatttggtactggctacctggagccttatcttctgaagggttttaaagaatggccaattagctgagaagaattatctaatcaattagtgatgtctgccatggatgcagtagaggaaagtggtggtacaagtgccatgattgattagcaatgtctgcactggatacggaaaaaagaaggtgcttgcaggtttacagtgtatatgtgggctattgaagagccctctgagctcggttgctagcaggagagcatgcccatattggcttactttgtctgccacagacacagacagagggagttgggacatgcatgctatggggaccctcttgttggacacctaattggatgcctcttcatgagaggcctccttttcttcaccttttatgctgcactcctcccctagtttacacatcttgatgctgtggctcagtttgccttcctgaatttttattgggtccctgttttctctcctaacatgctgagattctgcatccccacagcctaaactgagccagtggccaaacaaccgtgctcagcctgtttctctctgccctctagagcaaggcccaccaggtccatccaggaggctctcctgacctcaagtccaacaacagtgtccacactagtcaaggttcagcccagaaaacagaaagcactctaggaatcttaggcagaaagggattttatctaaatcactggaaaggctggaggagcagaaggcagaggccaccactggactattggtttcaatattagaccactgtagccgaatcagaggccagagagcagccactgctactgctaatgccaccactacccctgccatcactgccccacatggacaaaactggagtcgagacctaggttagattcctgcaaccacaaacatccatcagggatggccagctgccagagctgcgggaagacggatcccacctccctttcttagcagaatctaaattacagccagacctctggctgcagaggagtctgagacatgtatgattgaatgggtgccaagtgccagggggcggagtccccagcagatgcatcctggccatctgttgcgtggatgagggagtgggtctatctcagaggaaggaacaggaaacaaagaaaggaagccactgaacatcccttctctgctccacaggagtgccttagacagcctgactctccacaaaccactgttaaaacttacctgctaggaatgctagattgaatgggatgggaagagccttccctcattattgtcattcttggagagaggtgagcaaccaagggaagctcctctgattcacctagaacctgttctctgccgtctttggctcagcctacagagactagagtaggtgaagggacagaggacagggcttctaatacctgtgccatattgacagcctccatccctgtcccccatcttggtgctgaaccaacgctaagggcaccttcttagactcacctcatcgatactgcctggtaatccaaagctagaactctcaggaccccaaactccacctcttggattggccctggctgctgccacacacatatccaagagctcagggccagttctggtgggcagcagagacctgctctgccaagttgtccagcagcagagtggccctggcctgggcatcacaagccagtgatgctcctgggaagaccaggtggcaggtcgcagttgggtaccttccattcccaccacacagactctgggcctccccgcaaaatggctccagaattagagtaattatgagatggtgggaaccagagcaactcaggtgcatgatacaaggagaggttgtcatctgggtagggcagagaggagggcttgctcatctgaacaggggtgtatttcattccaggccctcagtctttggcaatggccaccctggtgttggcatattggccccactgtaacttttgggggcttcccggtctagccacaccctcggatggaaagacttgactgcataaagatgtcagttctccctgagttgattgataggcttaatggtcaccctaaaaacacccacatatgcttttcgatggaaccaggtaagttgacgctaaagttcttatggaaaaatacacacgcaatagctaggaaaacacagggaaagaagagttctgagcagggcctagtcttagccaatattaaaacatactatgaagcctctgatacttaaacagcatggcgctggtacgtaaatagaccaatgcagttaggtggctctttccaagactctggggaaaaaagtagtaaaaagctaaatgcaatcaatcagcaattgaaagctaagtgagagagccagagggcctccttggtggtaaaagagggttgcatttcttgcagccagaaggcagagaaagtgaagaccaagtccagaactgaatcctaagaaatgcaggactgcaaagaaattggtgtgtgtgtgtgtgtgtgtgtgtgtgtgtgtgtttaatttttaaaaagtttttattgagatacaagtcaataccataaagctctcacccttctaaagtgtacaattcagtggtgtgagtatattcataagatttatacttggtgtctattcataagacttatatccagcatattcataactagagccatatcacagatgcattcatcataataattccagacattttcatcaccctaaaaggaaaccctgaaacccattagcagtcattccccattcctccaacccattctctccctaatccctagaaaccaccaatctgctgtgtatttcatctattgccaacatttcatataaatggcatcatacaaaaaaaaaaaaaaaaaaaaa

SEQ ID NO: 29 - DNA, 3264 bp, Homo sapiens
  CDS (1)..(3264)
atggacttgtacagcaccccggccgctgcgctggacaggttcgtggccagaaggctgcagccgcggaaggagttcgtagagaaggcgcggcgcgctctgggcgccctggccgctgccctgagggagcgcgggggccgcctcggtgctgctgccccgcgggtgctgaaaactgtcaagggaggctcctcgggccggggcacagctctcaagggtggctgtgattctgaacttgtcatcttcctcgactgcttcaagagctatgtggaccagagggcccgccgtgcagagatcctcagtgagatgcgggcatcgctggaatcctggtggcagaacccagtccctggtctgagactcacgtttcctgagcagagcgtgcctggggccctgcagttccgcctgacatccgtagatcttgaggactggatggatgttagcctggtgcctgccttcaatgtcctgggtcaggccggctccggcgtcaaacccaagccacaagtctactctaccctcctcaacagtggctgccaagggggcgagcatgcggcctgcttcacagagctgcggaggaactttgtgaacattcgcccagccaagttgaagaacctaatcttgctggtgaagcactggtaccaccaggtgtgcctacaggggttgtggaaggagacgctgcccccggtctatgccctggaattgctgaccatcttcgcctgggagcagggctgtaagaaggatgctttcagcctagccgaaggcctccgaactgtcctgggcctgatccaacagcatcagcacctgtgtgttttctggactgtcaactatggcttcgaggaccctgcagttgggcagttcttgcagcggcagcttaagagacccaggcctgtgatcctggacccagctgaccccacatgggacctggggaatggggcagcctggcactgggatttgctagcccaggaggcagcatcctgctatgaccacccatgctttctgagggggatgggggacccagtgcagtcttggaaggggccgggccttccacgtgctggatgctcaggtttgggccaccccatccagctagaccctaaccagaagacccctgaaaacagcaagagcctcaatgctgtgtacccaagagcagggaggaaacctccctcatgcccagctcctggccccactggggcagccagcatcgtcccctctgtgccgggaatggccttggacctgtctcagatccccaccaaggagctggaccgcttcatccaggaccacctgaagccgagcccccagttccaggagcaggtgaaaaaggccatcgacatcatcttgcgctgcctccatgagaactgtgttcacaaggcctcaagagtcagtaaagggggctcatttggccggggcacagacctaagggatggctgtgatgttgaactcatcatcttcctcaactgcttcacggactacaaggaccaggggccccgccgcgcagagatccttgatgagatgcgagcgcagctagaatcctggtggcaggaccaggtgcccagcctgagccttcagtttcctgagcagaatgtgcctgaggctctgcagttccagctggtgtccacagccctgaagagctggacggatgttagcctgctgcctgccttcgatgctgtggggcagctcagttctggcaccaaaccaaatccccaggtctactcgaggctcctcaccagtggctgccaggagggcgagcataaggcctgcttcgcagagctgcggaggaacttcatgaacattcgccctgtcaagctgaagaacctgattctgctggtgaagcactggtaccgccaggttgcggctcagaacaaaggaaaaggaccagcccctgcctctctgcccccagcctatgccctggagctcctcaccatctttgcctgggagcagggctgcaggcaggattgtttcaacatggcccaaggcttccggacggtgctggggctcgtgcaacagcatcagcagctctgtgtctactggacggtcaactatagcactgaggacccagccatgagaatgcaccttcttggccagcttcgaaaacccagacccctggtcctggaccccgctgatcccacctggaacgtgggccacggtagctgggagctgttggcccaggaagcagcagcgctggggatgcaggcctgctttctgagtagagacgggacatctgtgcagccctgggatgtgatgccagccctcctttaccaaaccccagctggggaccttgacaagttcatcagtgaatttctccagcccaaccgccagttcctggcccaggtgaacaaggccgttgataccatctgttcatttttgaaggaaaactgcttccggaattctcccatcaaagtgatcaaggtggtcaagggtggctcttcagccaaaggcacagctctgcgaggccgctcagatgccgacctcgtggtgttcctcagctgcttcagccagttcactgagcagggcaacaagcgggccgagatcatctccgagatccgagcccagctggaggcatgtcaacaggagcggcagttcgaggtcaagtttgaagtctccaaatgggagaatccccgcgtgctgagcttctcactgacatcccagacgatgctggaccagagtgtggactttgatgtgctgccagcctttgacgccctaggccagctggtctctggctccaggcccagctctcaagtctacgtcgacctcatccacagctacagcaatgcgggcgagtactccacctgcttcacagagctacaacgggacttcatcatctctcgccctaccaagctgaagagcctgatccggctggtgaagcactggtaccagcagtgtaccaagatctccaaggggagaggctccctacccccacagcacgggctggaactcctgactgtgtatgcctgggagcagggcgggaaggactcccagttcaacatggctgagggcttccgcacggtcctggagctggtcacccagtaccgccagctctgtatctactggaccatcaactacaacgccaaggacaagactgttggagacttcctgaaacagcagcttcagaagcccaggcctatcatcctggatccggctgacccgacaggcaacctgggccacaatgcccgctgggacctgctggccaaggaagctgcagcctgcacatctgccctgtgctgcatgggacggaatggcatccccatccagccatggccagtgaaggctgctgtgtga

SEQ ID NO: 30 - protein, 1087 aa, Homo sapiens [sequence omitted]

SEQ ID NO: 31 - DNA, 20 bp, artificial sequence
  OAS1/pro1 F-primer
ccaggagttcgagaccagac

SEQ ID NO: 32 - DNA, 20 bp, artificial sequence
  OAS1/pro1 F-primer
ggtgaggcataggggatttt

SEQ ID NO: 33 - DNA, 20 bp, artificial sequence
  OAS1/pro3 F-primer
ccttgaacccaggaagttga

SEQ ID NO: 34 - DNA, 20 bp, artificial sequence
  OAS1/pro4 F-primer
ctttttgcagtgggcatgta

SEQ ID NO: 35 - DNA, 20 bp, artificial sequence
  OAS1/exon1 F-primer
tttgcaaaaggaaagtgcaa

SEQ ID NO: 36 - DNA, 20 bp, artificial sequence
  OAS1/exon2 F-primer
agcatccattttcccatctg

SEQ ID NO: 37 - DNA, 20 bp, artificial sequence
  OAS1/exon3 F-primer
ggatcaggaatggacctcaa

SEQ ID NO: 38 - DNA, 20 bp, artificial sequence
  OAS1/exon4 F-primer
aatgaatgagcctggattcg

SEQ ID NO: 39 - DNA, 20 bp, artificial sequence
  OAS1/exon5 F-primer
gagcccttcctcatgttctg

SEQ ID NO: 40 - DNA, 20 bp, artificial sequence
  OAS1/exon6-1 F-primer
agctggggcttgttagtcct

SEQ ID NO: 41 - DNA, 20 bp, artificial sequence
  OAS1/exon6-2 F-primer
gctcctcagtgagctggtgt

SEQ ID NO: 42 - DNA, 21 bp, artificial sequence
  OAS1/exon6-3 F-primer
cggccatgtcattgtttttat

SEQ ID NO: 43 - DNA, 20 bp, artificial sequence
  OAS2/pro1 F-primer
aaagcacaggctttggagtc

SEQ ID NO: 44 - DNA, 20 bp, artificial sequence
  OAS2/pro2 F-primer
ttgggggagctcagtcttta

SEQ ID NO: 45 - DNA, 20 bp, artificial sequence
  OAS2/pro3 F-primer
tttctggctcacacacttgg

SEQ ID NO: 46 - DNA, 20 bp, artificial sequence
  OAS2/pro4 F-primer
cgatgtgctcaaggacagac

SEQ ID NO: 47 - DNA, 20 bp, artificial sequence
  OAS2/exon1 F-primer
cctcccatcctaccattcac

SEQ ID NO: 48 - DNA, 20 bp, artificial sequence
  OAS2/exon2 F-primer
atgagcatcccaatcaggtc

SEQ ID NO: 49 - DNA, 21 bp, artificial sequence
  OAS2/exon3 F-primer
tgaccgattaagcaagagtcg

SEQ ID NO: 50 - DNA, 20 bp, artificial sequence
  OAS2/exon4 F-primer
taagagaggtccctgctcca

SEQ ID NO: 51 - DNA, 20 bp, artificial sequence
  OAS2/exon5 F-primer
gaggggcttgatgtcgtaga

SEQ ID NO: 52 - DNA, 20 bp, artificial sequence
  OAS2/exon6 F-primer
ttgtgtgccactccaaactc

SEQ ID NO: 53 - DNA, 20 bp, artificial sequence
  OAS2/exon7 F-primer
cagaacttggccttggaaac

SEQ ID NO: 54 - DNA, 20 bp, artificial sequence
  OAS2/exon8 F-primer
ggagatgctccctgtgtctt

SEQ ID NO: 55 - DNA, 20 bp, artificial sequence
  OAS2/exon9 F-primer
tccaagctgcagagtgtgag

SEQ ID NO: 56 - DNA, 20 bp, artificial sequence
  OAS2/exon10-1 F-primer
cgcggcctctagaaagttac

SEQ ID NO: 57 - DNA, 20 bp, artificial sequence
  OAS2/exon10-2 F-primer
aggcaggtaaccccagattc

SEQ ID NO: 58 - DNA, 20 bp, artificial sequence
  OAS2/exon10-3 F-primer
tggttctctccaccaggttc

SEQ ID NO: 59 - DNA, 20 bp, artificial sequence
  OAS2/exon11-1 F-primer
gtagggaagaggtggccaag

SEQ ID NO: 60 - DNA, 20 bp, artificial sequence
  OAS2/exon11-2 F-primer
gtcttgccttgctgaactcc

SEQ ID NO: 61 - DNA, 20 bp, artificial sequence
  OAS2/exon11-3 F-primer
ccagccaaccctttcattag

SEQ ID NO: 62 - DNA, 20 bp, artificial sequence
  OAS3/pro1 F-primer
tattgacccttctgcgttcc

SEQ ID NO: 63 - DNA, 20 bp, artificial sequence
  OAS3/pro2 F-primer
ggagagaggacctgggagtt

SEQ ID NO: 64 - DNA, 20 bp, artificial sequence
  OAS3/pro3 F-primer
ccagccagacaactttccag

SEQ ID NO: 65 - DNA, 20 bp, artificial sequence
  OAS3/exon1 F-primer
aagtgggtgtcaggtccaag

SEQ ID NO: 66 - DNA, 20 bp, artificial sequence
  OAS3/exon2 F-primer
acagtctctccccagcacac

SEQ ID NO: 67 - DNA, 20 bp, artificial sequence
  OAS3/exon3 F-primer
aaagctccaggctctcttcc

SEQ ID NO: 68 - DNA, 20 bp, artificial sequence
  OAS3/exon4 F-primer
ggctgaagctaccagtgagg

SEQ ID NO: 69 - DNA, 20 bp, artificial sequence
  OAS3/exon5 F-primer
tcgaggctcagagagggtaa

SEQ ID NO: 70 - DNA, 20 bp, artificial sequence
  OAS3/exon6 F-primer
ggcacctttttcaactctgc

SEQ ID NO: 71 - DNA, 20 bp, artificial sequence
  OAS3/exon7 F-primer
catgcagtcgacctttgtca

SEQ ID NO: 72 - DNA, 21 bp, artificial sequence
  OAS3/exon8 F-primer
ccactgtgcagttactggatg

SEQ ID NO: 73 - DNA, 20 bp, artificial sequence
  OAS3/exon9 F-primer
ctaggcaaaagccacgtctg

SEQ ID NO: 74 - DNA, 20 bp, artificial sequence
  OAS3/exon10 F-primer
cccaatagctttccaaccaa

SEQ ID NO: 75 - DNA, 20 bp, artificial sequence
  OAS3/exon11 F-primer
cctcagaacctacccaccag

SEQ ID NO: 76 - DNA, 21 bp, artificial sequence
  OAS3/exon12 F-primer
tgacttgtccaaggtcacaca

SEQ ID NO: 77 - DNA, 20 bp, artificial sequence
  OAS3/exon13 F-primer
atcccagaccacccttagga

SEQ ID NO: 78 - DNA, 20 bp, artificial sequence
  OAS3/exon14 f-primer
aaagttttggggttgctttg

SEQ ID NO: 79 - DNA, 20 bp, artificial sequence
  OAS3/exon15 F-primer
tctagcccctgcaaagtgtt

SEQ ID NO: 80 - DNA, 20 bp, artificial sequence
  OAS3/exon16-1 F-primer
atctgtggtgccaaaggaag

SEQ ID NO: 81 - DNA, 20 bp, artificial sequence
  OAS3/exon16-2 F-primer
cctcccatggcttacacact

SEQ ID NO: 82 - DNA, 20 bp, artificial sequence
  OAS3/exon16-3 F-primer
caggagagcatgcccatatt

SEQ ID NO: 83 - DNA, 20 bp, artificial sequence
  OAS3/exon16-4 F-primer
gagattctgcatccccacag

SEQ ID NO: 84 - DNA, 20 bp, artificial sequence
  OAS3/exon16-5 F-primer
ctctggctgcagaggagtct

SEQ ID NO: 85 - DNA, 20 bp, artificial sequence
  OAS3/exon16-6 F-primer
aacgctaagggcaccttctt

SEQ ID NO: 86 - DNA, 20 bp, artificial sequence
  OAS3/exon16-7 F-primer
caccctggtgttggcatatt

SEQ ID NO: 87 - DNA, 20 bp, artificial sequence
  OAS1/pro1 R-primer
tgcagggacagtcacagaac

SEQ ID NO: 88 - DNA, 20 bp, artificial sequence
  OAS1/pro1 R-primer
ataggaggtggggcttgact

SEQ ID NO: 89 - DNA, 20 bp, artificial sequence
  OAS1/pro3 R-primer
cctttgtcctttagccagca

SEQ ID NO: 90 - DNA, 20 bp, artificial sequence
  OAS1/pro4 R-primer
tgtcaatggcatggttgatt

SEQ ID NO: 91 - DNA, 20 bp, artificial sequence
  OAS1/exon1 R-primer
gaaaccaccatcccagaaga

SEQ ID NO: 92 - DNA, 20 bp, artificial sequence
  OAS1/exon2 R-primer
cccaccctgctttagagaga

SEQ ID NO: 93 - DNA, 20 bp, artificial sequence
  OAS1/exon3 R-primer
ggctcctctctccacctctt

SEQ ID NO: 94 - DNA, 20 bp, artificial sequence
  OAS1/exon4  R-primer
tacaatgatgggcaacagga

SEQ ID NO: 95 - DNA, 20 bp, artificial sequence
  OAS1/exon5 R-primer
ataaatggcctttggcaaga

SEQ ID NO: 96 - DNA, 20 bp, artificial sequence
  OAS1/exon6-1 R-primer
tggaatgaagtgaggctgtg

SEQ ID NO: 97 - DNA, 20 bp, artificial sequence
  OAS1/exon6-2 R-primer
tccatgaggacagggatttt

SEQ ID NO: 98 - DNA, 20 bp, artificial sequence
  OAS1/exon6-3 R-primer
actgccagagtggagatgct

SEQ ID NO: 99 - DNA, 20 bp, artificial sequence
  OAS2/pro1 R-primer
tgagcccaggtacccagata

SEQ ID NO: 100 - DNA, 20 bp, artificial sequence
  OAS2/pro2 R-primer
ctgacttgctcagggacaca

SEQ ID NO: 101 - DNA, 20 bp, artificial sequence
  OAS2/pro3 R-primer
agcagggaaaccaaaactga

SEQ ID NO: 102 - DNA, 20 bp, artificial sequence
  OAS2/pro4 R-primer
ccatttcccattgctctcag

SEQ ID NO: 103 - DNA, 20 bp, artificial sequence
  OAS2/exon1 R-primer
gtcacacagctctggagcaa

SEQ ID NO: 104 - DNA, 20 bp, artificial sequence
  OAS2/exon2 R-primer
gagagcgagtccagggtaga

SEQ ID NO: 105 - DNA, 22 bp, artificial sequence
  OAS2/exon3 R-primer
tgttgcactttactggatgtca

SEQ ID NO: 106 - DNA, 20 bp, artificial sequence
  OAS2/exon4 R-primer
ggacctcccctctttcagac

SEQ ID NO: 107 - DNA, 20 bp, artificial sequence
  OAS2/exon5 R-primer
ctctgccaacaccctgattt

SEQ ID NO: 108 - DNA, 20 bp, artificial sequence
  OAS2/exon6 R-primer
taactcctggcttcccttcc

SEQ ID NO: 109 - DNA, 20 bp, artificial sequence
  OAS2/exon7 R-primer
cccatgcttggaaaagagaa

SEQ ID NO: 110 - DNA, 20 bp, artificial sequence
  OAS2/exon8 R-primer
gggttagtgatgggaacgtc

SEQ ID NO: 111 - DNA, 20 bp, artificial sequence
  OAS2/exon9 R-primer
gcccctgctaggttttatcc

SEQ ID NO: 112 - DNA, 20 bp, artificial sequence
  OAS2/exon10-1 R-primer
ggttgtcactgggagaggag

SEQ ID NO: 113 - DNA, 20 bp, artificial sequence
  OAS2/exon10-2 R-primer
gcacaagagagcctccaaac

SEQ ID NO: 114 - DNA, 20 bp, artificial sequence
  OAS2/exon10-3 R-primer
ggatggatcctagctccaca

SEQ ID NO: 115 - DNA, 21 bp, artificial sequence
  OAS2/exon11-1 R-primer
gactggaaggaaaaaggagga

SEQ ID NO: 116 - DNA, 20 bp, artificial sequence
  OAS2/exon11-2 R-primer
aaggctaaagcccaaggaaa

SEQ ID NO: 117 - DNA, 20 bp, artificial sequence
  OAS2/exon11-3 R-primer
cacatgcttggtctttctgg

SEQ ID NO: 118 - DNA, 20 bp, artificial sequence
  OAS3/pro1 R-primer
gcacacctgagctctcttcc

SEQ ID NO: 119 - DNA, 20 bp, artificial sequence
  OAS3/pro2 R-primer
cgaccatggtgtaggtgcta

SEQ ID NO: 120 - DNA, 20 bp, artificial sequence
  OAS3/pro3 R-primer
tcggatttctggtttcgttt

SEQ ID NO: 121 - DNA, 20 bp, artificial sequence
  OAS3/exon1 R-primer
tgtcaagcgggcttaagagt

SEQ ID NO: 122 - DNA, 20 bp, artificial sequence
  OAS3/exon2 R-primer
cccaggtcaaacagcaagtt

SEQ ID NO: 123 - DNA, 21 bp, artificial sequence
  OAS3/exon3 R-primer
tgcacaagggacataaacaga

SEQ ID NO: 124 - DNA, 20 bp, artificial sequence
  OAS3/exon4 R-primer
ctaatgtgcatctgggctga

SEQ ID NO: 125 - DNA, 20 bp, artificial sequence
  OAS3/exon5 R-primer
gcctcccaatgtgttgagat

SEQ ID NO: 126 - DNA, 20 bp, artificial sequence
  OAS3/exon6 R-primer
ccagcaaatcatccttcctg

SEQ ID NO: 127 - DNA, 22 bp, artificial sequence
  OAS3/exon7 R-primer
tcaatgcttggtcaagtaggag

SEQ ID NO: 128 - DNA, 20 bp, artificial sequence
  OAS3/exon8 R-primer
aagcaaaggaggggagatgt

SEQ ID NO: 129 - DNA, 20 bp, artificial sequence
  OAS3/exon9 R-primer
ggaagcagagtgaggagtgg

SEQ ID NO: 130 - DNA, 20 bp, artificial sequence
  OAS3/exon10 R-primer
gctgggacgtagcaatctgt

SEQ ID NO: 131 - DNA, 20 bp, artificial sequence
  OAS3/exon11 R-primer
ggacatatctgggccctctc

SEQ ID NO: 132 - DNA, 20 bp, artificial sequence
  OAS3/exon12 R-primer
aaaggggccctcttctttag

SEQ ID NO: 133 - DNA, 19 bp, artificial sequence
  OAS3/exon13 R-primer
ctccaccaacccaggatct

SEQ ID NO: 134 - DNA, 20 bp, artificial sequence
  OAS3/exon14 R-primer
attgaccgtttgtccctcag

SEQ ID NO: 135 - DNA, 20 bp, artificial sequence
  OAS3/exon15 R-primer
atgggaaaatggaagcacag

SEQ ID NO: 136 - DNA, 20 bp, artificial sequence
  OAS3/exon16-1 R-primer
gcaggaggaaagatgtgagc

SEQ ID NO: 137 - DNA, 20 bp, artificial sequence
  OAS3/exon16-2 R-primer
catagcatgcatgtcccaac

SEQ ID NO: 138 - DNA, 19 bp, artificial sequence
  OAS3/exon16-3 R-primer
gagagcctcctggatggac

SEQ ID NO: 139 - DNA, 20 bp, artificial sequence
  OAS3/exon16-4 R-primer
tagacccactccctcatcca

SEQ ID NO: 140 - DNA, 20 bp, artificial sequence
  OAS3/exon16-5 R-primer
gccctgagctcttggatatg

SEQ ID NO: 141 - DNA, 20 bp, artificial sequence
  OAS3/exon16-6 R-primer
gggtgtttttagggtgacca

SEQ ID NO: 142 - DNA, 21 bp, artificial sequence
  OAS3/exon16-7 R-primer
agaagggtgagagctttatgg